(19) United States
(12) Patent Application Publication
(10) Pub. No.: US 2020/0164000 A1
(43) Pub. Date: May 28, 2020

(54) COMPOSITIONS AND METHODS FOR TREATING DISORDERS RELATED TO A GUT DYSBIOSIS

(71) Applicant: CRESTOVO HOLDINGS LLC, Somerville, MA (US)

(72) Inventors: Marina SANTIAGO, Cambridge, MA (US); Sonia TIMBERLAKE, Brookline, MA (US)

(73) Assignee: CRESTOVO HOLDINGS LLC, Somerville, MA (US)

(21) Appl. No.: 16/667,301

(22) Filed: Oct. 29, 2019

Related U.S. Application Data
(60) Provisional application No. 62/751,887, filed on Oct. 29, 2018.

(51) Int. Cl. A61K 35/74; C12Q 1/06

(52) U.S. Cl. CPC A61K 35/74; C12Q 1/06

(57) ABSTRACT

This application provides a microbiome disruption index (MDI) for determining or monitoring the taxonomic structure of an intestinal or fecal microbiota of a subject. Disclosed herein is the use of an MDI in the diagnosis, treatment or prognosis of disorders, diseases, conditions or indications caused by, associated with, or related to a gut dysbiosis. Further disclosed is the use of MDI to assess the efficacy of a microbiome-based therapy.

This application claims priority to U.S. Provisional Application No. 62/751,887, filed Oct. 29, 2018, which is incorporated by reference in its entirety herein.

### BACKGROUND

Fecal bacteria extracted from the stool of a human stool donor can be administered to a patient to alleviate the symptoms of certain infections and disorders, for example recurrent Clostridium difficile infections. Such fecal microbial transplants (FMTs) are typically administered following the onset of clinical symptoms associated with gut dysbiosis. However, pre-symptomatic underlying disruptions or perturbations in the microbiome of the gut that lead to susceptibility to acute or chronic disorders can go undetected. Further, if such undetected disruptions or perturbations in microbial community structure are present in the fecal microbiota of donor stool administered to patients, then FMT efficacy or safety can be compromised.

### SUMMARY

In an aspect, this disclosure provides a method of treating a patient having a disorder associated with a gut dysbiosis, the method comprising: receiving a value of a microbiome disruption index (MDI) corresponding to a fecal microbiota of said patient, wherein said MDI is representative of a divergence in microbial taxonomic diversity between said fecal microbiota of said patient and fecal microbiota of one or more healthy subjects, wherein said one or more healthy subjects do not have said disorder; and administering a therapeutic composition comprising a preparation of live non-pathogenic fecal bacteria to said patient based on said value of said MDI.

In another aspect, this disclosure provides a method, comprising: administering a therapeutic composition comprising a preparation of live non-pathogenic fecal bacteria to a patient having a disorder associated with a gut dysbiosis; receiving a value of a microbiome disruption index (MDI) corresponding to a fecal microbiota of said patient following said administering, wherein said MDI is representative of a divergence in microbial taxonomic diversity between said fecal microbiota of said patient and fecal microbiota of one or more healthy subject that do not have said disorder; and administering a second dose of said therapeutic composition to said patient based on said value of said MDI being greater than a threshold value.

In an aspect, this disclosure provides a method of treating a subject having a disorder, the method comprising administering to said subject a therapeutic composition comprising a preparation of uncultured fecal bacteria, wherein said administering is based on a value of a microbiome disruption index (MDI) corresponding to a fecal microbiota of said subject, wherein said MDI is representative of a divergence in bacterial diversity between said fecal microbiota and fecal microbiota of one or more healthy individuals, wherein said disorder is selected from the group consisting of an infectious disease, an autoimmune disease, an allergic disease, and a neurological disease, and wherein said one or more healthy individuals do not have said disorder.

### BRIEF DESCRIPTION OF THE DRAWINGS

FIG. 1: Data is collected from a multicenter retrospective study. 16S sequencing on samples collected from 84 patients at 6 centers is performed. These patients all have rCDI. Some of these patients receive an allogenic FMT from a universal donor at OpenBiome, while others receive an autologous FMT as a control. Samples from before and after FMT are obtained for all patients. Patients are assessed for CDI clinical cure 8 weeks post-FMT. Samples are assessed retrospectively for VRE colonization using the OpGen MDRO testing service.

FIG. 2: Allogenic FMT is more effective at preventing recurrence of infection than autologous FMT. 91% of patients who receive allogenic FMT do not experience CDI recurrence. 63% of patients who receive autologous FMT do not experience CDI recurrence. The difference between these groups is significant by Fisher's exact test.

FIG. 3: FMT decolonizes VRE at 8 weeks. At baseline, ˜20-40% of rCDI patients are colonized with VRE. However, after 6 weeks, 92% of those initially colonized are decolonized in the group receiving allogenic FMT, while only 43% of those initially colonized in the group receiving autologous FMT is decolonized. The difference between these groups is significant by Fisher's exact test.

FIGS. 4A-D: A microbiome disruption index is developed. FIG. 4A: Alpha diversity, as measured by the Shannon Index is highest for the stool donors, similarly high for patient samples post-FMT, and lowest for patient samples pre-FMT. FIG. 4B: Beta diversity, as measured by the Jensen-Shannon Divergence, compared to donors is highest for patient samples pre-FMT and are overall much lower for patient samples post-FMT. FIG. 4C: Combining measures of alpha and beta diversity into microbiome disruption index allows one to identify different types of community dysbiosis. The MDI is calculated by multiplying the average difference in alpha diversity between a sample and each of the stool donors by the average beta diversity between a sample and each of the stool donors. FIG. 4D: This calculation results in an MDI where undisrupted communities are generally found between an MDI of 0 and 1, and disrupted communities have an MDI of greater than 1.

FIGS. 5A-D: The MDI can predict different patient characteristics. ROC curves are used to determine whether the MDI could predict (FIG. 5A) which patient samples are from patients infected with CDI (before or after FMT) and (FIG. 5B) of the post-FMT samples, which patients receive autologous or allogenic FMT. The AUC values show that the MDI can reliably predict both. FIG. 5C: There is no difference in MDI pre-FMT between those patients who are and are not cured post allogenic FMT. FIG. 5D: This is reflected in a ROC curve with a low AUC value.

FIGS. 6A-B: The MDI does not predict which samples are colonized with VRE. FIG. 6A: The MDI is not able to predict which subjects were colonized with VRE. FIG. 6B: However, it is observed that the average relative abundance of Enterococcus in VRE-positive patients pre-FMT is 100× lower post allogenic-FMT than post-autologous FMT, suggesting that significant community changes are occurring

FIG. 7: Proteobacterial abundance is much higher in VRE positive samples than in VRE negative samples or in stool donors. OTUs from three taxonomic clades (Proteobacteria, Bacteroidales, and Clostridiales) are identified as being enriched in VRE positive samples (before or after FMT) using the group significance command in Qiime. Of these three, only Proteobacteria are enriched in VRE positive samples.

FIGS. 8A-B: Enterobacteriaceae abundance predicts VRE colonization in the pre-FMT samples. FIG. 8A: Of the three different Proteobacterial groups tested (all Proteobacteria, specific clades are identified in the group significance test, and all Enterobacteriaceae family members), the Enterobacteriaceae family best predict VRE colonization. Line 801 shows the total Proteobacterial abundance predicts VRE colonization (AUC=0.893; p<0.001). Line 802 shows the abundance of specific Proteobacteria taxa predicts VRE colonization (AUC=0.919; p<0.001). Line 803 shows the abundance of Enterobacteriaceae predicts VRE colonization (AUC=0.924; p<0.001). FIG. 8B: In fact, the relative abundance of all Enterobacteriaceae in these samples is similar to that of all Proteobacteria, suggesting that the observed predictive effect is driven by Enterobacteriaceae.

FIG. 9 is a flowchart showing an exemplary protocol for a method of administering non-pathogenic fecal bacteria to a subject based on a value of a microbiome disruption index (MDI).

FIG. 10 is a flowchart showing an exemplary protocol for a method of manufacturing a pharmaceutical composition comprising a fecal microbiota of a stool donor.

FIGS. 11A-B: MDI predicts which patients develop bloodstream infection. FIG. 11A: Patients who go on to develop bloodstream infection generally have an MDI greater than 1 on or before the day they receive their stem cell transplant. FIG. 11B: This fact allows us to predict which patients will develop a bloodstream infection, with a statistically significant AUC=0.722.

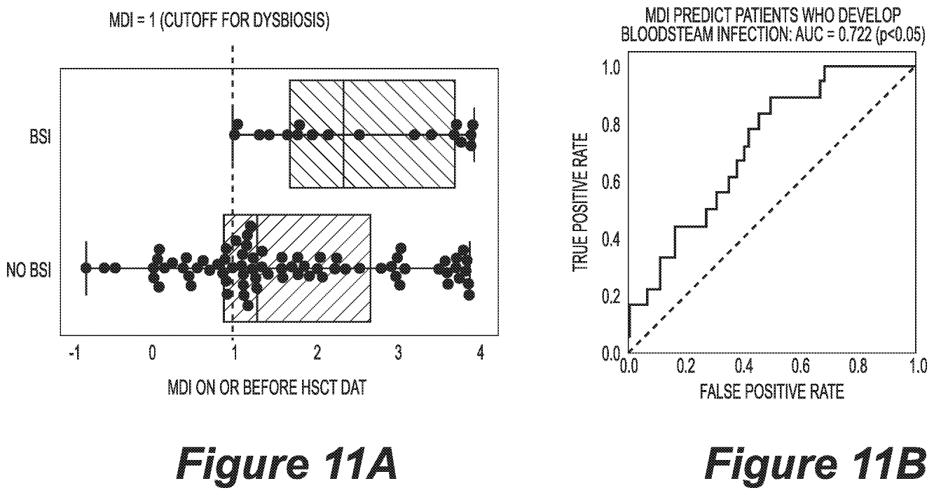

FIG. 12 is a block diagram of an example computer device for implementing a MDI-based microbiome prediction.

### DETAILED DESCRIPTION

Unless defined otherwise herein, terms are to be understood according to conventional usage by those of ordinary skill in the relevant art.

All publications, patents, and patent applications mentioned in this specification are herein incorporated by reference to the same extent as if each individual publication, patent, or patent application was specifically and individually indicated to be incorporated by reference.

As used in the description of the disclosure and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

The terms “about” and “approximately” as used herein when referring to a measurable value such as percentages, density, volume and the like, is meant to encompass variations of 20%, 10%, 5%, 1%, 0.5%, or even 0.1% of the specified amount.

As used herein, the term “substantially”, when used to modify a quality, generally allows certain degree of variation without that quality being lost. For example, in certain aspects such degree of variation can be less than 0.1%, about 0.1%, about 0.2%, about 0.3%, about 0.4%, about 0.5%, about 0.6%, about 0.7%, about 0.8%, about 0.9%, about 1%, between 1-2%, between 2-3%, between 3-4%, between 4-5%, or greater than 5%.

Where a range of values is provided, it is understood that each intervening value, between the upper and lower limit of that range and any other stated or intervening value in that stated range is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either both of those included limits are also included in the disclosure.

As used herein, the term “relative abundance” refers to relative representation of an organism of a particular kind (e.g., a bacterial strain, species, or genus) relative to all organisms of similar nature in a certain community (e.g., a preparation of uncultured fecal bacteria or a bacterial mixture). Relative abundance is calculated by dividing the number of an organism of a particular kind by the total number of all organisms of similar nature in a certain community. In an aspect, relative abundance is measured by qPCR comparing PCR products generated with 16S primers targeting specific bacterial strains of interest against PCR products generated with universal primers targeting all 16S sequences. See e.g., Chu, N., et al., “Profiling living bacteria informs preparation of fecal microbiota transplantations.” PLoS One 12(1): 1-16 (2017). In another aspect, the relative abundance is measured based on the number of sequence reads detected via high-throughput sequencing. Unless specified otherwise, a bacterial relative abundance mentioned herein is measured via high-throughput sequencing. In a further aspect, propidium monoazide (PMA) is used to differentiate between viable and dead fecal microbes as shown in Chu et al., PLoS One 12(1): 1-16 (2017).

As used herein, the term “treating” refers to (i) completely or partially inhibiting a disease, disorder or condition, for example, arresting its development; (ii) completely or partially relieving a disease, disorder or condition, for example, causing regression of the disease, disorder and/or condition; or (iii) completely or partially preventing a disease, disorder or condition from occurring in a patient that may be predisposed to the disease, disorder and/or condition, but has not yet been diagnosed as having it. Similarly, “treatment” refers to both therapeutic treatment and prophylactic or preventative measures.

As used herein, “therapeutically effective amount” or “pharmaceutically active dose” refers to an amount of a composition which is effective in treating the named disease, disorder or condition.

As used herein, “microbiota,” and “flora” refer to a community of microbes that live in or on a subject's body, both sustainably and transiently, including eukaryotes, archaea, bacteria, and viruses (including bacterial viruses (i.e., phage)). A “fecal microbiota” or “fecal microbiota preparation” refers to a community of microbes present in or prepared from a subject's feces. Typically a pharmaceutical composition described herein is prepared by incorporating such a fecal microbiota into the composition without culturing the fecal microbiota after its purification from a stool. Herein “uncultured fecal bacteria” or a “preparation of uncultured fecal bacteria” refer to a preparation comprising multiple non-pathogenic viable bacterial strains that have been harvested, extracted or purified from one or more stool samples, without culturing the strains (e.g. in culturing medium). Such a preparation of uncultured fecal bacteria can also be referred to as a collection of uncultured fecal bacteria or a population of uncultured fecal bacteria.

In some aspects, a preparation of uncultured fecal bacteria comprises non-selected fecal bacteria. Herein “non-selected fecal bacteria” refers to a collection of viable fecal bacterial strains (e.g., present in a fecal microbiota) extracted from one or more stool samples without subjecting the extracted bacteria to environmental conditions that intentionally select for a particular type, state or taxonomic category of bacteria (e.g., by deliberate removal of certain strains of bacteria, treatment of the bacteria with an agent such as ethanol or chloroform, or culturing). Such non-selected fecal bacteria can comprise bacterial strains in proportional content to corresponding bacterial strains in a fecal or intestinal microbiota of a normal healthy human. Steps taken to non-selectively extract fecal bacteria from a stool sample can include, for example, homogenization and filtering of the stool sample to separate the fecal bacterial strains from non-cellular stool material such as fiber and rough particulate matter, as well as, for example, eukaryotic host cells and viruses. Herein typically a non-selected fecal bacterial preparation can be prepared in either aerobic or anaerobic conditions, or a combination thereof. In certain aspects, a preparation of non-selected fecal bacteria comprises all or substantially all of the bacteria of a fecal microbiota of a stool sample. In certain aspects, a preparation of non-selected fecal bacteria comprises all or substantially all of the strains of a fecal microbiota of a stool sample. In certain aspects, a preparation of non-selected fecal bacteria comprises all or substantially all of the species of a fecal microbiota of a stool sample. In certain aspects, a preparation of non-selected fecal bacteria comprises all or substantially all of the genera of a fecal microbiota of a stool sample. In certain aspects, a preparation of non-selected fecal bacteria comprises all or substantially all of the phyla of a fecal microbiota of a stool sample. Therefore, such non-selective fecal microbiota can substantially resemble microbial constituents and the bacterial population structure found in such fecal sample. A non-selected fecal microbiota refers to a community or mixture of fecal microbes derived from a donor's fecal sample without selection and substantially resembling microbial constituents and population structure found in such fecal sample.

As used herein, “bacteria,” “bacterium,” and “archaea” refer to single-celled prokaryotes that lack membrane bound nuclei and lack organelles.

As used herein, “colony forming units” (cfu) refers to an estimate of the number of viable microorganism cells in a given sample. The number of CFUs can be assessed by counting the number of colonies on an agar plate as in standard methods for determining the number of viable bacterial cells in a sample.

As used herein, “viable” means possessing the ability to multiply. The viability of bacterial populations can be monitored as a function of the membrane integrity of the cell. Cells with a compromised membrane are considered to be dead or dying, whereas cells with an intact membrane are considered live. For example, SYTO 9 and propidium iodide are used to stain and differentiate live and dead bacteria. See Stocks, Cytometry A. 2004 October; 61(2):189-95. Cell viability can also be evaluated via molecular viability analyses, e.g., a PCR-based approach, which can differentiate nucleic acids associated with viable cells from those associated with inactivated cells. See Cangelosi and Mescheke, Appl Environ Microbiol. 2014 October; 80(19): 5884-5891.

As used herein, “fecal bacteria” refers to bacteria that can be found in fecal matter.

In an aspect, a preparation of uncultured fecal bacteria comprises at least 2, 5, 10, 20, 30, 40, 50, 100, 200, 300, 400, 500, or 600 bacterial species or strains. In another aspect, a preparation of uncultured fecal bacteria comprises between 2 and 5, 5 and 10, 10 and 20, 20 and 30, 30 and 40, 40 and 50, 50 and 60, 60 and 100, 100 and 200, 200 and 300, 300 and 400, 400 and 500, or 500 and 600 bacterial species or strains.

In an aspect, a preparation of uncultured fecal bacteria and/or non-selected fecal bacteria does not comprise an antibiotic resistant population of bacteria.

In another aspect, the preparation of a composition comprising uncultured fecal bacteria can involve steps that select for a particular, type, state, or taxonomic category of bacteria (e.g., by deliberate removal of certain strains of bacteria, treatment of the population with a selective agent such as ethanol or chloroform, and/or screening of the bacteria for the ability to produce a metabolite at or above a threshold level).

Herein uncultured fecal bacteria are distinguished from a single, purified strain of bacteria such as a bacterial isolate. As used herein, “bacterial isolate” refers to an isolated group of substantially genetically identical bacterial cells generated by proliferation via binary fission from a single predecessor bacterial cell (e.g., by culturing the bacteria). Typically, a bacterial isolate is originally isolated as a single cell or genetically pure group of cells, for example, as a single colony on solid culture media or via serial dilutions in liquid culture, and thereafter archived (e.g. as a frozen stock) to provide a consistent and stable source for the isolate. Once isolated, in some aspects, a bacterial isolate can be grown as a pure culture of cells; in other aspects, multiple bacterial isolates can be grown simultaneously in the same vessel as a mixed culture. The term “substantially genetically identical” refers to the very high (e.g. >99.9%) genetic identity shared by different cells in uncontaminated pure compositions of bacterial isolates, owing to their proliferation from a common predecessor, but accounts for minor genetic dissimilarity between cells due to accumulations of relatively rare mutations. Generally, a bacterial isolate is synonymous with a pure culture of bacterial cells. Typically, herein a bacterial isolate consists of non-pathogenic bacteria. In an aspect, a bacterial isolate can be a probiotic, or an ingredient in a probiotic.

As used herein, the term “bacterial cocktail”, sometimes called a “bacterial consortium” or “synthetic bacterial mixture”, refers to an engineered mixture of bacteria comprising a defined consortium of multiple bacterial isolates. The term “defined consortium of multiple bacterial isolates” means that the bacterial cocktail contains two or more bacterial isolates, and that the identity of each bacterial isolate in the cocktail is known, and thus the cocktail can be consistently produced (e.g. by combining isolated bacterial strains) to have a stable composition and properties across separate batches. Herein “identity” of a bacterial isolate can refer to any characteristic of the isolate that uniquely identifies the isolate as different from one or more other bacterial isolates or bacterial strains. Examples of identifying characteristics of a bacterial isolate include nucleotide sequences such as a 16S rRNA sequence, the sequence of one or more coding or non-coding regions of a nucleic acid, and entire genome sequences, levels of gene expression, physiological or metabolic traits, or anatomical traits such as staining pattern or cell wall characteristics.

As used herein, “bacterial mixture” refers to an engineered composition comprising viable bacterial cells. In some aspects, a bacterial mixture comprises one or more non-pathogenic bacterial isolates. In some aspects, a bacterial mixture comprises a preparation of uncultured fecal bacteria. In some aspects, a bacterial mixture comprises both of one or more non-pathogenic bacterial isolates and a preparation of uncultured fecal bacteria.

As used herein, “isolated” or “purified” refers to a bacterium or other entity or substance that has been (1) separated from at least some of the components with which it was associated when initially produced (whether in nature or in an experimental setting), and/or (2) produced, prepared, purified, and/or manufactured by the hand of man. Isolated or purified bacteria can be separated from at least about 10%, about 20%, about 30%, about 40%, about 50%, about 60%, about 70%, about 80%, about 90%, or more of the other components with which they were initially associated.

As used herein, “cytotoxic” activity or bacterium includes the ability to kill a bacterial cell, such as a pathogenic bacterial cell. A “cytostatic” activity or bacterium includes the ability to inhibit, partially or fully, growth, metabolism, and/or proliferation of a bacterial cell, such as a pathogenic bacterial cell.

As used herein, the terms “pathogen” and “pathogenic” in reference to a bacterium or any other organism or entity includes any such organism or entity that is capable of causing or affecting a disease, disorder or condition of a host organism containing the organism or entity.

As used herein, “spore” or a population of “spores” includes bacteria (or other single-celled organisms) that are generally viable, more resistant to environmental influences such as heat and bacteriocidal agents than vegetative forms of the same bacteria, and typically capable of germination and out-growth. “Spore-formers” or bacteria “capable of forming spores” are those bacteria containing the genes and other necessary abilities to produce spores under suitable environmental conditions.

As used herein, a “combination” of two or more bacteria includes the physical co-existence of the two bacteria, either in the same material or product or in physically connected products, as well as the temporal co-administration or co-localization of the two bacteria.

As used herein, “subject” refers to any animal subject including humans, laboratory animals (e.g., primates, rats, mice), livestock (e.g., cows, sheep, goats, pigs, turkeys, chickens), and household pets (e.g., dogs, cats, rodents, etc.). Preferred subjects are human subjects. The human subject may be a pediatric, adult or a geriatric subject. In some aspects, the terms “patient” and “subject” are used interchangeably. The subject or patient may be healthy, or may be suffering from an infection due to a gastrointestinal pathogen or may be at risk of developing or transmitting to others an infection due to a gastrointestinal pathogen. As used herein, a “healthy subject” refers to a subject who has no history of gastrointestinal disease or condition. Exemplary healthy subjects include the screened donors described in paragraph [0235] below.

As used herein, “Shannon Diversity Index” refers to a diversity index that accounts for abundance and evenness of species present in a given community using the formula

$$H = - {\sum }_{i = 1}^{R} {p}_{i} ln {p}_{i}$$

where H is Shannon Diversity Index, R is the total number of species in the community, and pi is the proportion of R made up of the ith species. Higher values indicate diverse and equally distributed communities, and a value of 0 indicates only one species is present in a given community. For further reference, see Shannon and Weaver, (1949) The mathematical theory of communication. The University of Illinois Press, Urbana. 117 pp.

As used herein, “antibiotic” refers to a substance that is used to treat and/or prevent bacterial infection by killing bacteria, inhibiting the growth of bacteria, or reducing the viability of bacteria.

As used herein, an “intermittent dosing schedule” means that that a therapeutic composition is administered for a period of time followed by a period of time (a treatment period) where treatment with such therapeutic composition is withheld (a rest period). Intermittent dosing regimens can be expressed as treatment period in days or weeks/rest period in days or weeks. For example, a 4/1 intermittent dosing schedule refers to an intermittent dosing schedule where the treatment period is four weeks/days and the rest period is one week/day.

As used herein, a “continuous dosing schedule” refers to a dosing schedule where a therapeutic composition is administered during a treatment period without a rest period. Throughout the treatment period of a continuous dosing schedule, a therapeutic composition can be administered, for example, daily, or every other day, or every third day. On a day when a therapeutic composition is administered, it can be administered in a single dose, or in multiple doses throughout the day.

As used herein, “dosing frequency” refers to the frequency of administering doses of a therapeutic composition in a given time. Dosing frequency can be indicated as the number of doses per a given time, for example, once per day, once a week, or once in two weeks.

As used herein, “dosing interval” refers to the amount of time that elapses between multiple doses being administered to a subject.

Disclosed herein is a method of treating a subject having a condition, disorder or disease related to or caused by a dysbiosis in an intestinal microbiota. Herein “dysbiosis” refers to an imbalance or perturbation in the structure of an intestinal microbial community relative to a healthy intestinal gut flora. In certain embodiments such imbalance or perturbation can predispose the subject to development of a particular condition, disorder, or disease, for example an infection, autoimmune disease, allergy or neurological disease.

In certain embodiments, a method of treatment comprises screening a subject for a disrupted or perturbated fecal microbiota by determining or obtaining a value for a microbiome disruption index (MDI) corresponding the subject's fecal microbiota, and administering live non-pathogenic fecal bacteria to the subject based on the value of the MDI. In other embodiments, a method of treatment comprises receiving an MDI value corresponding to a subject's fecal microbiota, and administering live non-pathogenic fecal bacteria to the subject based on the value of the MDI.

Further disclosed herein is a method of manufacturing a pharmaceutical composition comprising live non-pathogenic fecal bacteria of a stool donor comprising obtaining or receiving an MDI value corresponding to the fecal microbiota of the stool donor, and incorporating the live non-pathogenic fecal bacteria of the stool donor into the pharmaceutical composition based on the value of the MDI.

In an aspect, a method comprises administering or manufacturing a preparation of uncultured fecal bacteria comprising an entire or substantially complete fecal microbiota from stool of a donor. In one aspect, uncultured fecal bacteria comprise an isolated or purified population of live non-pathogenic fecal bacteria. In a further aspect, uncultured fecal bacteria comprise a substantially complete fecal microbiota preparation from a single donor. In another aspect, a pharmaceutical composition described herein comprises a bacterial mixture comprising one or more live, non-pathogenic, bacterial isolates and live, non-pathogenic, purified or extracted, uncultured fecal bacteria.

Herein the term “microbiome disruption index” or “MDI” refers to a measure of the divergence, if any, of the taxonomic structure of an intestinal or fecal microbiota of a subject from the taxonomic structure of an intestinal or fecal microbiota from one or more healthy individuals. The term “taxonomic structure” encompasses both qualitative distinctions, i.e., differences in the types of bacteria (e.g., genus, species or strain) between the microbiota of a subject and microbiota of one or more healthy individuals, as well as quantitative distinctions, i.e., differences in the quantity or abundance of a particular type of bacteria between the microbiota of a subject and microbiota of one or more healthy individuals.

In certain aspects, an MDI embodies a measure of the difference in species richness, or number of taxa, between a fecal microbiota of a subject and fecal microbiota of one or more healthy subjects. For example, the MDI can include a measure of divergence in alpha diversity, if any, between a fecal microbiota of a subject and the fecal microbiota of multiple healthy individuals. In certain embodiments, the measure of divergence in alpha diversity is derived using fecal microbiota from multiple healthy individuals by determining an average difference between the species richness of the subject's fecal microbiota and that of each healthy individual. In one aspect, alpha diversity is measured using Shannon's diversity index. In one aspect, alpha diversity is measured using Simpson's index. Herein the term “species richness” can incorporate both the types of microbial strains in a fecal microbiota as well as the abundance of an individual strain or strains. In an aspect, a fecal microbiota with a greater number of strains is considered to have a greater species richness than a second fecal microbiota with a lower number of strains. In another aspect, a fecal microbiota having microbial strains showing a more diverse taxonomic structure have a greater species richness than a second fecal microbiota with a less diverse taxonomic structure. In another aspect, a measure of alpha diversity and/or species richness is representative of an abundance of one or more bacterial strains in the fecal microbiota.

In certain aspects, an MDI embodies a beta diversity of a fecal microbiota of a subject relative to a fecal microbiota of one or more healthy individuals. For example, an MDI can incorporate a measure of the difference in a taxonomic abundance profile between a fecal microbiota of a subject and fecal microbiota of one or more healthy subjects. In certain embodiments, the beta diversity represents an average beta diversity between a fecal microbiota of a subject and that of each healthy individual. In one aspect, beta diversity is a measure of Jensen-Shannon divergence.

In certain aspects, an MDI embodies both a measure of divergence in alpha diversity and beta diversity, if any, between a fecal microbiota of a subject and the fecal microbiota of multiple healthy individuals. For example, an MDI can be the product of a measure of divergence in alpha diversity and the beta diversity between a fecal microbiota of a subject and the fecal microbiota of one or more healthy individuals. In such embodiments, MDI can be represented by the following formula:

MDI=(α_{healthy}−α_{subject})=β_{healthy-patient}

where α_{healthy} represents the alpha diversity of the healthy individual

α_{subject} represents the alpha diversity of the patient

β_{healthy-patient} represents the beta diversity.

As will be understood, low alpha diversity of a subject's fecal microbiota relative to that of a healthy individual will lead to a relatively high value for alpha diversity divergence. Likewise, beta diversity will be high if the subject's fecal microbiota has, for example, a narrow taxonomic abundance profile relative to the profile of a healthy subject. Accordingly, the MDI of a subject increases as the subject's fecal microbiota has a lower alpha diversity (i.e. used to calculate the divergence in alpha diversity compared to a healthy individual) and/or a higher beta diversity. Where both the divergence in alpha diversity and the beta diversity are high relative to a healthy subject, the MDI will correspondingly be higher. As shown in the Examples below, in one embodiment the MDI value can be used as a threshold marker beyond which a subject can be considered at risk for gut dysbiosis and disorders, conditions or diseases related to gut dysbiosis. In certain aspects, a subject having a disrupted gut microbiome community has an MDI of greater than 1, whereas a subject having an undisrupted gut microbiome community has an MDI of less than 1.

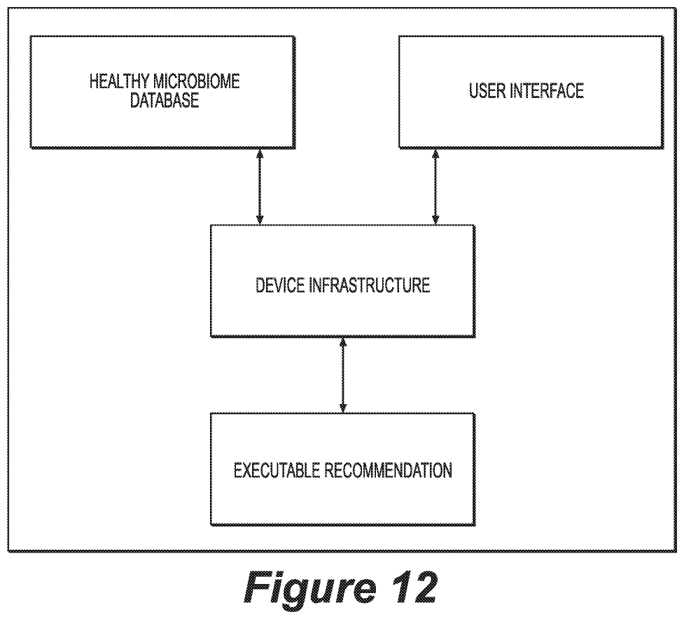

An MDI is typically determined by implementing one or more of the determination steps described herein (e.g., a determination of alpha diversity and/or beta diversity) on a computer or computing device, such as that exemplified at FIG. 12.

In certain embodiments, a subject's MDI can be used as a risk factor predictive of the existence of a dysbiosis in the intestinal microbiota of the subject or susceptibility to such dysbiosis. Thus the MDI advantageously provides a means to identify perturbations in a subject's gut microbial community before clinical symptoms become apparent.

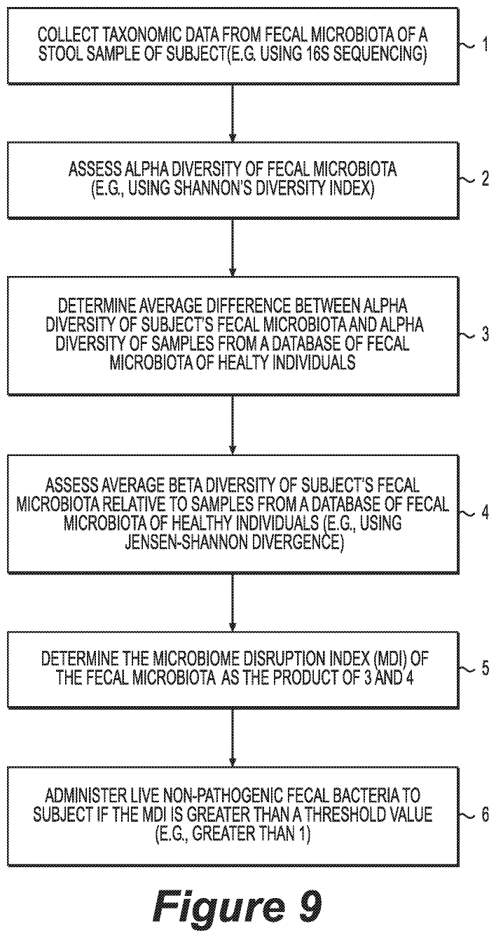

FIG. 9 shows an exemplary protocol for administering live non-pathogenic bacteria to a subject based on a value of an MDI corresponding to the subject's fecal microbiota. At step 1, taxonomic data is collected from the fecal microbiota of a stool sample of the subject. For example, DNA can be extracted from bacteria isolated from the stool sample and the 16S regions of the DNA can be sequenced to obtain a library of the types and abundance of strains making up the intestinal microbiome of the subject. At step 2, alpha diversity of the fecal microbiota of the subject is determined, for example using Shannon's diversity index. At step 3, the alpha diversity determined at step 2 is compared with the alpha diversity of fecal microbiota isolated from one or more healthy individuals to provide a measure of divergence of the subject's alpha diversity from that of the healthy individual(s). In some embodiments, the alpha diversity of the healthy individual(s) can be stored in and received from a database. Where alpha diversity of multiple healthy individuals is used to determine the divergence, the difference in alpha diversity between the subject and each healthy individual can be determined, and then the average difference can be used as the final measure of alpha divergence. At step 4, the beta diversity of the subject's fecal microbiota relative to one or more healthy individuals is determined, for example using Jensen-Shannon divergence. Where beta diversity is determined relative to multiple healthy individuals, the beta diversity relative to each healthy individual can be averaged to obtain a final value. In some embodiments, beta diversity can be determined using healthy individuals' fecal microbiota profiles stored in and received from a database. In one aspect, divergence in alpha diversity and beta diversity are determined using fecal microbiota profiles from the same healthy individuals. In alternative aspects, divergence in alpha diversity and beta diversity are determined using fecal microbiota profiles from different healthy individuals. At step 5, the MDI of the subject's fecal microbiota is determined as the product of the divergence in alpha diversity (i.e. between the fecal microbiota of the subject and one or more healthy individuals) and the beta diversity. At step 6, the MDI value is used as a basis for the administration of live non-pathogenic fecal bacteria (i.e., obtained from the fecal microbiota of a healthy donor) to the subject if the MDI exceeds a threshold value. In one embodiment, the subject is administered live non-pathogenic fecal bacteria from a healthy donor if the subject's MDI exceeds a value of 1.

In an aspect, the MDI can be used as a basis to select a subject as a candidate for a fecal microbiota transplant. For example, a method of treating a subject for a disorder associated with gut dysbiosis can comprise determining an MDI of a fecal microbiota of the subject; and administering live fecal bacteria to the subject if the MDI exceeds a threshold value (e.g. 1). In an aspect, an MDI can be used as the basis for screening a population of subjects (i.e. 2 or more individuals) for gut dysbiosis. For example, an MDI corresponding to the fecal microbiota of each subject of the population of subjects can be determined, and live fecal microbiota can be administered to each subject having an MDI exceeding a threshold value (e.g. 1).

In certain embodiments, an MDI can be used in a method of manufacture as a risk factor to avoid or lower the risk of incorporation of fecal bacteria into a pharmaceutical composition that may present a health risk. Thus the MDI advantageously provides a means to identify potential undesired perturbations in a candidate donor's gut microbial community before clinical symptoms become apparent in the candidate donor, thereby providing MDI as an additional screening factor that can be used to screen out a candidate donor's stool having a fecal microbiota with an MDI that is above or below a threshold value (e.g. greater than 1). Thus, the MDI can complement other screening or risk factors such as disease history and presence of pathogens in stool in determining whether fecal bacteria from a candidate donor's stool are suitable for incorporation into a pharmaceutical product for administration to a subject in need thereof. In certain embodiments, a candidate donor's fecal bacteria can be rejected for inclusion in a drug product where the MDI exceeds a threshold value (e.g. 1). Alternatively, the candidate donor's stool can be included in a drug product where the MDI is less than a threshold value (e.g. 1).

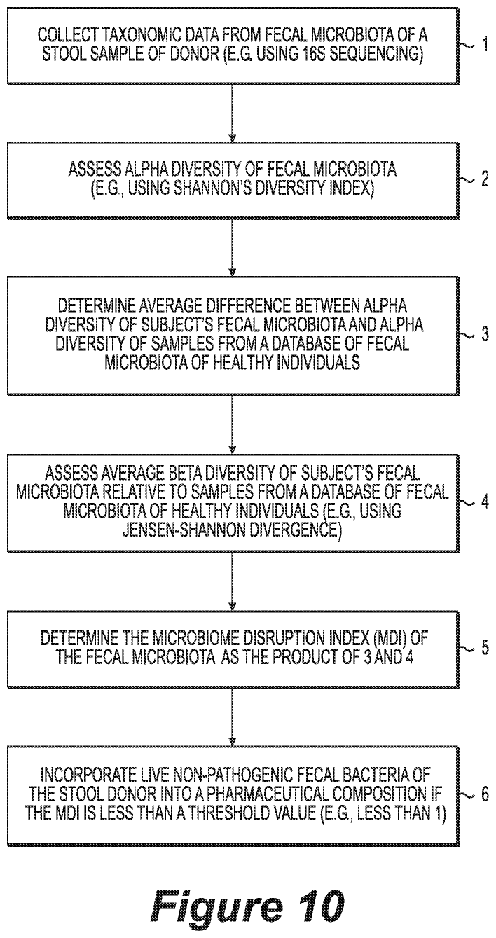

FIG. 10 shows an exemplary protocol for manufacturing a pharmaceutical composition based on the value of an MDI of a stool donor's fecal microbiota. At step 1, taxonomic data is collected from the fecal microbiota of a stool sample of a candidate donor. For example, DNA can be extracted from bacteria isolated from the stool sample and the 16S regions of the DNA can be sequenced to obtain a library of the types and abundance of strains making up the intestinal microbiome of the candidate donor. At step 2, alpha diversity of the fecal microbiota of the donor is determined, for example using Shannon's diversity index. At step 3, the alpha diversity determined at step 2 is compared with the alpha diversity of fecal microbiota isolated from healthy individuals to provide a measure of divergence of the alpha diversity of the candidate donor's fecal microbiota from that of one or more healthy individuals. In some embodiments, the alpha diversity of the healthy individuals can be stored in and received from a database. Where alpha diversity of multiple healthy individuals is used to determine the divergence, the difference in alpha diversity between the fecal microbiota of the candidate donor and each healthy individual can be determined, and then the average difference in alpha diversity can be used as the final measure of alpha diversity divergence. At step 4, the beta diversity of the candidate donor's fecal microbiota relative to one or more healthy individuals is determined, for example using Jensen-Shannon divergence. Where beta diversity is determined relative to multiple healthy individuals, the beta diversity relative to each healthy individual can be averaged to obtain a final value. At step 5, the MDI corresponding to the candidate donor's fecal microbiota is determined as the product of the divergence in alpha diversity (i.e. between the fecal microbiota of the candidate donor and one or more healthy individuals) and the beta diversity. At step 6, the MDI value is used as a basis to incorporate live fecal bacteria from a stool sample of the donor into a pharmaceutical composition if the MDI exceeds a threshold value. In one embodiment, the live fecal bacteria of the stool sample are incorporated into the pharmaceutical composition if the MDI exceeds a value of 1.

An MDI disclosed herein can be used as a basis (e.g., where the MDI exceeds a threshold value) to treat, prevent or inhibit a variety of disorders, diseases, conditions or indications caused by or related to a gut dysbiosis. Likewise, a pharmaceutical product incorporating live, non-pathogenic fecal bacteria extracted from a donor having a fecal microbiota with an acceptable MDI (e.g. less than a threshold value) can be administered to a recipient to treat a variety of disorders, diseases, conditions or indications. Non-limiting examples of disorders, diseases, conditions or indications contemplated herein as targets of pharmaceutical compositions comprising live, non-pathogenic fecal bacteria include Acne, AIDS Enteropathy, AIDS-related Gastroenteritis, Alopecia Totalis, Alzheimers Disease, Amyloidosis, Amyotrophic Lateral Sclerosis, Ankylosing Spondylitis, Anorexia, Antibiotic Associated Colitis, Asbergers Syndrome, Attention Deficit Disorder (ADD), Attention Deficit Hyperactivity Disorder (ADHD), Autism Spectrum Disorder (ASD), Behcet's Syndrome, Chronic Clostridium difficile Infection (CDI), Chronic constipation, Chronic Depression, Chronic Fatigue Syndrome (CFS), Chronic Idiopathic Pseudo Obstructive Syndrome, Chronic Inflammation Demyelinating Polyneuropathy, Chronic Nausea, Chronic Urticaria, Coeliac Disease, Collagenous Colitis, Colonic Polyps, Constipation Predominant FBD, Crohn's Disease, Cryptogenic Cirrhosis, Cyclic Vomiting, Dermatitis Herpetiformis, Diabetes, Familial Mediterranean Fever, Fatty Liver, Functional Bowel Disease (FBD), Gastro-oesophageal Reflux, Gillian-Barre Syndrome, Glomerulonephritis, Haemolytic Uraemic Syndrome, Halitosis, IBS constipation-predominant, IBS diarrhea/constipation alternating, IBS diarrhea-predominant, IBS pain-predominant, Idiopathic Thrombocytopenic Purpura (ITP), Idiopathic/Simple Constipation, Indeterminate Colitis, Inflammatory Bowel Disease (IBD), Irritable bowel syndrome (B S), Juvenile Diabetes Mellitus, Lyme Disease, Manic Depressive Illness, Metabolic Syndrome, Microscopic Colitis, Migraine, Mixed Cryoglobulinaemia, Mucous Colitis, Multiple Sclerosis, Myasthenia Gravis, NASH (Nonalcoholic Steatohepatitis), Non-Rheumatoid Arthritis, Non-Rheumatoid Factor Positive Arthritis, Non-ulcer Dyspepsia, Norwalk Viral Gastroenteritis, Obesity, Obsessive Compulsive Disorder, Pain Predominant FBD, Parkinson's Disease, Polyarteritis, Polyposis Coli, Primary Biliary Cirrhosis, Primary Clostridium difficile Infection (CDI), Primary Sclerosing Cholangitis (PSC), Pseudomembranous Colitis, Psychotic Disorders, Reiter's Syndrome, Relapsing Diverticulitis, Rett Syndrome, Rheumatoid Arthritis, Rosacea, Rotavirus Gastroenteritis, Sacroiliitis, Schizophrenia, Scleroderma, Sjogren's Syndome, Small Bowel Bacterial Overgrowth, Sudden Infant Death Syndrome (SIDS), Systemic Lupus Erythematosus, Ulcerative Colitis, Upper Abdominal FBD, Vasculitic Disorders, Viral Gastroenteritis, pre-diabetic syndrome, type I diabetes, type II diabetes, depression, schizophrenia, a mood disorder, Vancomycin Resistant Enterococci (VRE) infection, Methicillin Resistant Staphylococcus Aureus (MRSA) infection, an autoimmune disorder, an infection, an allergy or atopy and a neurological disorder.

In an aspect, an MDI disclosed here is used as a basis (e.g., where the MDI exceeds a threshold value) for the diagnosis or prognosis of disorders, diseases, conditions or indications caused by, associated with, or related to a gut dysbiosis. For example, an MDI can be used to detect a bloodstream infection in a subject or identify a subject at risk for a bloodstream infection. In an aspect, such a subject has undergone or is to undergo certain medical treatment or procedure (e.g., hematopoietic stem cell transplantation (HSCT)).

In another aspect, the present disclosure provides for methods for treating a subject in need thereof by administering to the subject a pharmaceutically active dose of a pharmaceutical composition comprising a preparation of uncultured fecal bacteria of a single donor (or blended from multiple donors). In another aspect, the administering is followed by testing the subject for an MDI to determine the efficacy of the pharmaceutically active dose of the pharmaceutical composition. In another aspect, the testing of the subject provides results (e.g., when an MDI is above a threshold value) to determine if the active dose of the pharmaceutical composition should be adjusted. In another aspect, the testing is followed by administration of a pharmaceutical composition comprising a preparation of uncultured fecal bacteria (e.g., from a single donor or comprising fecal bacteria blended from multiple donors).

In another aspect, disclosed herein is a method of decolonizing antibiotic-resistant bacteria from the intestine of a subject in need thereof, comprising administering live non-pathogenic fecal bacteria to said subject based on detection of a greater relative abundance of bacteria from the Proteobacteria phylum in a fecal microbiota of said subject compared to a fecal microbiota of a healthy individual. In an embodiment, the antibiotic-resistant bacteria comprise Vancomycin-Resistant Enterococcus. In an embodiment, the bacteria from the Proteobacteria phylum comprise Enterobacteriaceae. In an embodiment, the bacteria from the Proteobacteria phylum consist of Enterobacteriaceae. In an embodiment, the healthy individual is not colonized with Vancomycin-Resistant Enterococcus.

In an aspect, the present disclosure provides a method for treating a disorder, disease or condition related to or caused by a dysbiosis of an intestinal microbiota in a subject in need thereof, where the method comprises administering to the subject a pharmaceutically active dose of a therapeutic composition comprising live non-pathogenic bacteria. In one aspect, the method comprises administering daily to the subject a pharmaceutically active dose of a therapeutic composition comprising live non-pathogenic fecal bacteria. In one aspect, a therapeutic composition is administered to a patient in need thereof at least once daily or weekly for at least two consecutive days or weeks. In one aspect, a therapeutic composition is administered at least once daily or weekly for at least 3, 4, 5, 6, 7, 8, 9, 10, 11, 12, 13, 14, or 15 consecutive days or weeks. In another aspect, a therapeutic composition is administered at least once daily or weekly for at least 1, 2, 3, 4, 5, 6, 7, 8, 9, 10, 11, or 12 consecutive days or weeks. In one aspect, a therapeutic composition is administered at least once daily or weekly for at most 4, 5, 6, 7, 8, 9, 10, 11, 12, 13, 14, 15, 16, 17, 18, 19, or 20 consecutive days or weeks. In another aspect, a therapeutic composition is administered at least once daily or weekly for at most 1, 2, 3, 4, 5, 6, 7, 8, 9, 10, 11, or 12 consecutive weeks or months. In a further aspect, a therapeutic composition is administered at least once for at least 1, 2, 3, 4, 5, 6, 7, 8, 9, 10, 11, or 12 consecutive months or years, chronically for a subject's entire life span, or an indefinite period of time.

In one aspect, a therapeutic composition is administered to a subject having a disorder, disease or condition related to or caused by a dysbiosis of an intestinal microbiota at least twice daily or weekly for at least two consecutive days or weeks. In one aspect, a therapeutic composition is administered at least twice daily or weekly for at least 3, 4, 5, 6, 7, 8, 9, 10, 11, 12, 13, 14, or 15 consecutive days or weeks. In another aspect, a therapeutic composition is administered at least twice daily or weekly for at least 1, 2, 3, 4, 5, 6, 7, 8, 9, 10, 11, or 12 consecutive days or weeks. In one aspect, a therapeutic composition is administered at least twice daily or weekly for at most 4, 5, 6, 7, 8, 9, 10, 11, 12, 13, 14, 15, 16, 17, 18, 19, or 20 consecutive days or week. In another aspect, a therapeutic composition is administered at least twice daily or weekly for at most 1, 2, 3, 4, 5, 6, 7, 8, 9, 10, 11, or 12 consecutive weeks or months. In a further aspect, a therapeutic composition is administered at least twice for at least 1, 2, 3, 4, 5, 6, 7, 8, 9, 10, 11, or 12 consecutive months or years, chronically for a subject's entire life span, or an indefinite period of time.

In one aspect, a therapeutic composition is administered to a subject having a disorder, disease or condition related to or caused by a dysbiosis of an intestinal microbiota at least three times daily or weekly for at least two consecutive days or weeks. In one aspect, a therapeutic composition is administered at least three times daily or weekly for at least 3, 4, 5, 6, 7, 8, 9, 10, 11, 12, 13, 14, or 15 consecutive days or weeks. In another aspect, a therapeutic composition is administered at least three times daily or weekly for at least 1, 2, 3, 4, 5, 6, 7, 8, 9, 10, 11, or 12 consecutive days or weeks. In one aspect, a therapeutic composition is administered at least three times daily for at most 4, 5, 6, 7, 8, 9, 10, 11, 12, 13, 14, 15, 16, 17, 18, 19, or 20 consecutive days or weeks. In another aspect, a therapeutic composition is administered at least three times daily for at most 1, 2, 3, 4, 5, 6, 7, 8, 9, 10, 11, or 12 consecutive weeks or months. In a further aspect, a therapeutic composition is administered at least three times for at least 1, 2, 3, 4, 5, 6, 7, 8, 9, 10, 11, or 12 consecutive months or years, chronically for a subject's entire life span, or an indefinite period of time.

In one aspect, the present disclosure provides a method for treating a subject having a disorder, disease or condition related to or caused by a dysbiosis of an intestinal microbiota, where the method comprises administering orally to the subject a pharmaceutically active dose of a therapeutic composition comprising live, non-pathogenic, synthetic bacterial mixture or live, non-pathogenic, purified or extracted, fecal microbiota, where the dose is administered at a dosing schedule of at least once or twice daily or weekly for at least three consecutive days or weeks. In another aspect, a dose is administered at least once, twice, or three times daily or weekly for a period between 1 and 12 weeks, between 2 and 12 weeks, between 3 and 12 weeks, between 4 and 12 weeks, between 5 and 12 weeks, between 6 and 12 weeks, between 7 and 12 weeks, between 8 and 12 weeks, between 9 and 12 weeks, between 10 and 12 weeks, between 1 and 2 weeks, between 2 and 3 weeks, between 3 and 4 weeks, between 4 and 5 weeks, between 5 and 6 weeks, between 6 and 7 weeks, between 7 and 8 weeks, between 8 and 9 weeks, between 9 and 10 weeks, or between 10 and 11 weeks.

In one aspect, the present disclosure provides a method for treating a subject having a disorder, disease or condition related to or caused by a dysbiosis of an intestinal microbiota, where the method comprises a first dosing schedule followed by a second dosing schedule. In one aspect, a first dosing schedule comprises a treatment or induction dose. In one aspect, a first dosing schedule comprises a continuous dosing schedule. In another aspect, a second dosing schedule comprises a maintenance dose lower than or equal to a pharmaceutically active dose of a first dosing schedule. In another aspect, a second dosing schedule lasts for at least about 2, 4, 6, 8, 10, 12, 18, 24, 36, 48, 72, or 96 months. In one aspect, a second dosing schedule lasts permanently, for a treated subject's entire life span, or an indefinite period of time. In one aspect, a second dosing schedule is a continuous dosing schedule. In another aspect, a second dosing schedule is an intermittent dosing schedule. In a further aspect, a second dosing schedule is an intermittent dosing schedule comprising a treatment period of at least 1, 2, 3, 4, 5, 6, 7, 8, 9, 10, 11, 12, 13, or 14 days followed by a resting period of at least 1, 2, 3, 4, 5, 6, 7, 8, 9, 10, 11, 12, 13, or 14 days. In another aspect, a second dosing schedule comprises administering a second dose (e.g., a maintenance dose) every other day, every two days, or every 3, 4, 5, 6, 7, 8 days. In another aspect, a maintenance dose is administered for an extended period of time with or without titration (or otherwise changing the dosage or dosing schedule). In one aspect, the interval between a first and a second dosing schedule is at least about 1, 2, 3, 4, 5, 6, 7, 8, 9, 10, 11, or 12 weeks. In another aspect, a second dosing schedule (e.g., a maintenance dose) comprises a dosage about 2, 5, 10, 50, 100, 200, 400, 800, 1000, 5000 or more folds lower than the dosage used in a first dosing schedule (e.g., an initial treatment dose). In another aspect, a second dosing schedule (e.g., a maintenance dosing schedule) has an equal or lower dosing frequency than a first dosing schedule (e.g., an initial treatment dosing schedule). In another aspect, a second dosing schedule (e.g., a maintenance dosing schedule) has a higher dosing interval than a first dosing schedule (e.g., an initial treatment dosing schedule).

In one aspect, a first or second dosing schedule used in a method can be once-a-week, twice-a-week, or thrice-a-week. The term “once-a-week” means that a dose is administered once in a week, preferably on the same day of each week. “Twice-a-week” means that a dose is administered two times in a week, preferably on the same two days of each weekly period. “Thrice-a-week” means that a dose is administered three times in a week, preferably on the same three days of each weekly period.

In an aspect, a pharmaceutical composition described herein can be lyophilized or freeze dried and stored at ambient temperatures (e.g., room temperature), at a freezing temperature, or at between about 2° C. and 8° C. In an aspect, freeze-drying allows the majority of cells to remain viable, and produces a powdered form of the product that can be gently pulverized into a powder. The powder, or lyophilized or freeze-dried composition, then can be encapsulated into a carrier, e.g., a tablet, geltab, pill or capsule, e.g., an enteric-coated capsule, or placed into oil-filled capsules for ingestion. Alternatively, the freeze-dried or lyophilized product, or powder, can be reconstituted at ambient temperatures before delivery to an individual in e.g., a fluid, e.g., a sterile fluid, such as saline, a buffer or a media such as a fluid-glucose-cellobiose agar (RGCA) media.

For freeze-drying, in an aspect, bacteria are held in a liquid that will prevent bursting of cells on thawing. This can include various stabilizers, e.g., glycerol and appropriate buffers, and/or ethylene glycol. In an aspect, the cryoprotecting process uses final concentrations of stabilizer(s) of between about 10% and 80%, 20% and 70%, 30% and 60%, or 40% and 50%, depending on the stabilizer(s) used; in an aspect, this helps stabilize proteins by preventing formation of ice crystals that would otherwise destroy protein structures.

In an aspect, stabilizers that help reduce destruction of living bacteria include skim milk, erythritol, arabitol, sorbitol, glucose, fructose and other polyols. Polymers such as dextran and polyethylene glycol can also be used to stabilize bacterial cells.

In an aspect, manufacturing a pharmaceutical composition can comprise steps of: (1) coating the exterior of a dissociated capsule (i.e., comprising separate capsule body and capsule cap) with the exterior enteric coating, (2) filling the capsule body with a bacterial mixture (e.g., comprising a preparation of uncultured fecal bacteria), and (3) closing the capsule cap over the capsule body, thereby encapsulating the bacterial mixture in the enteric-coated capsule.

Optionally, manufacturing a pharmaceutical composition can comprise steps of: (1) coating the exterior of a dissociated capsule (i.e., comprising separate capsule body and capsule cap) with the exterior enteric coating, (2) coating the interior of the dissociated capsule with an interior coating, (3) filling the capsule body with a bacterial mixture (e.g., comprising a preparation of uncultured fecal bacteria), and (4) closing the capsule cap over the capsule body, thereby encapsulating the bacterial mixture in the dual-coated capsule.

Alternately, manufacturing a pharmaceutical composition can comprise step of: (1) coating the interior of the dissociated capsule (i.e., comprising separate capsule body and capsule cap) with an interior coating, (2) coating the exterior of a dissociated capsule with the exterior enteric coating, (3) filling the capsule body with a bacterial mixture (e.g., comprising a preparation of uncultured fecal bacteria), and (4) closing the capsule cap over the capsule body, thereby encapsulating the bacterial mixture in the dual-coated capsule.

In an aspect, one or more additional therapeutic agents can be included in a pharmaceutical composition, and encapsulated by the capsule.

In an aspect, the bodies and caps of gelatin capsules (e.g., size #00) are separated. An exterior enteric coating suspension is prepared by dispersing one or more enteric coating polymers along with other components in a solution. The exterior enteric coating suspension is applied to the exterior of separated capsule bodies and caps, e.g., using a fluid bed Wurster column coater, Fluid Bed Coater, or an equivalent). The capsules are fluidized in the product bowl and the exterior enteric coating suspension is sprayed to produce the outer coating to a target of between about 2 mg/cm2 and 6 mg/cm2, e.g., 3 mg/cm2. After completion of this step, the capsules are set to dry, e.g., between about 8 hours and 24 hours. After drying, exemplary capsules are weighed to calculate weight gain from the exterior enteric coating. Capsules can be inspected for irregularities.

In an aspect, EUDRAGIT® S100 (poly(methacrylic acid, methylmethacrylate)), starch, triethyl citrate, and PlasACRYL™ T20 are dissolved in a solution of water, ethanol, and n-butanol, mixed, and then charged to a suitable spraying device. The solution is then spray coated on the outer surface of the capsule bodies and capsule caps to a target weight gain. The capsule bodies and capsule caps are allowed to dry for about 8 hours to about 24 hours, or longer, e.g., for a week, a month, or more, before further procession, e.g., filling with a bacterial mixture.

In an aspect, it may be desirable to provide an amount of the bacterial mixture to a capsule's cap in addition to providing the composition in the capsule's body. In this aspect, more of the composition will be included in a capsule and/or less air will be contained in a closed capsule.

In an aspect, the interior surface of a capsule comprises an internal coating.

Any of the above-described compositions and materials (e.g., bacterial mixtures, inner coatings, capsules, and outer coatings) can be combined into a pharmaceutical composition described herein. A skilled artisan would know how to select an inner coating; capsule, and outer coating according to his/her present need, which could be based, for example, on a specific bacterial isolate(s) incorporated into a bacterial mixture of the composition and/or the desired delivery location in a subject (e.g., in the colon or small intestine, including the ileum, jejunum or duodenum) of a component of the bacterial mixture (e.g. comprising a preparation of uncultured fecal bacteria, a bacterial isolate and/or an additional therapeutic agent).

Additional relevant teachings are disclosed in WO 2007122374, which is hereby incorporated herein by reference in its entirety.

In an aspect, during the manufacture of a pharmaceutical composition, a pharmaceutically-acceptable cryoprotectant, lyoprotectant, binder, disintegrant, filler, preservative, acid suppressant, antacid, H2 antagonist, and proton pump inhibitor, or combination thereof can be mixed into the pharmaceutical composition (e.g., comprising a bacterial mixture) to promote desirable properties.

In an aspect, the pharmaceutical composition comprises a surface active agent. Surface active agents suitable for use include, but are not limited to, any pharmaceutically acceptable, non-toxic surfactant. Classes of surfactants suitable for use include, but are not limited to, polyethoxylated fatty acids, PEG-fatty acid diesters, PEG-fatty acid mono- and di-ester mixtures, polyethylene glycol glycerol fatty acid esters, alcohol-oil transesterification products, polyglycerized fatty acids, propylene glycol fatty acid esters, mixtures of propylene glycol esters-glycerol esters, mono- and diglycerides, sterol and sterol derivatives, polyethylene glycol sorbitan fatty acid esters, polyethylene glycol alkyl ethers, sugar esters, polyethylene glycol alkyl phenols, polyoxyethylene-olyoxypropylene block copolymers, sorbitan fatty acid esters, lower alcohol fatty acid esters, ionic surfactants, and mixtures thereof. In some aspects, compositions can comprise one or more surfactants including, but not limited to, sodium lauryl sulfate, polysorbate 20, polysorbate 40, poly-sorbate 60, polysorbate 80, and triethyl citrate.

In an aspect, the pharmaceutical composition comprises pharmaceutically acceptable plasticizers to obtain the desired mechanical properties such as flexibility and hardness. Such plasticizers include, but are not limited to, triacetin, citric acid esters, triethyl citrate, phthalic acid esters, dibutyl sebacate, cetyl alcohol, polyethylene glycols, polysorbates or other plasticizers.

In another aspect, the pharmaceutical composition comprises one or more application solvents. Some of the more common solvents that can be used to apply, for example, a delayed-release coating composition include isopropyl alcohol, acetone, methylene chloride and the like.

In yet another aspect, the pharmaceutical composition comprises one or more alkaline materials. Alkaline material suitable for use in compositions include, but are not limited to, sodium, potassium, calcium, magnesium and aluminum salts of acids such as phosphoric acid, carbonic acid, citric acid and other aluminum/magnesium compounds. In addition, the alkaline material can be selected from antacid materials such as aluminum hydroxides, calcium hydroxides, magnesium hydroxides and magnesium oxide.

Besides inert diluents, the orally administered compositions can also include adjuvants such as sweetening, flavoring, and perfuming agents.

In various aspects, the pharmaceutical compositions are formulated for systemic or local delivery. In an aspect, administration is systemic. In another aspect, it may be desirable to administer locally to the area in need of treatment.

Various methods can be used to formulate and/or deliver a pharmaceutical composition (e.g., comprising a bacterial mixture and/or additional therapeutic agent) described herein to a location of interest. For example, the pharmaceutical compositions can be formulated for delivery to the GI tract. The GI tract includes organs of the digestive system such as mouth, esophagus, stomach, small intestine, duodenum, jejunum, ileum, large intestine and rectum and includes all subsections thereof (e.g. the small intestine may include the duodenum, jejunum and ileum; the large intestine may include the colon transversum, colon descendens, colon ascendens, colon sigmoidenum and cecum). For example, the compositions can be formulated for delivery of one or more active agents to one or more of the stomach, small intestine, large intestine and rectum, or any subsection thereof (e.g. duodenum, jejunum and ileum, colon transversum, colon descendens, colon ascendens, colon sigmoidenum and cecum). In some aspects, the compositions described herein can be formulated for delivery of one or more active agents to the upper or lower GI tract. In an aspect, a composition can be administered to a subject, by, for example, directly or indirectly contacting the mucosal tissues of the GI tract with the composition.

In various aspects, the administration of the pharmaceutical compositions is into the GI tract via, for example, oral delivery, nasogastral tube, intestinal intubation (e.g. an enteral tube or feeding tube such as, for example, a jejunal tube or gastro-jejunal tube, etc.), direct infusion (e.g., duodenal infusion), endoscopy, colonoscopy, or enema.

In one aspect, a method comprises administering a pharmaceutical composition orally, by enema, or via rectal suppository. In one aspect, a pharmaceutical composition administered herein is formulated as an enteric coated (and/or acid-resistant) capsule or microcapsule, or formulated as part of or administered together with a food, a food additive, a dairy-based product, a soy-based product or a derivative thereof, a jelly, a gelatin-based chewable (e.g., gummy), flavored liquid, ice block, ice cream, or a yogurt. In another aspect, a pharmaceutical composition administered herein is formulated as an acid-resistant enteric coated capsule. A pharmaceutical composition can be provided as a powder for sale in combination with a food or drink. A food or drink can be a dairy-based product or a soy-based product. In another aspect, a food or food supplement contains enteric-coated and/or acid-resistant microcapsules containing a pharmaceutical composition.

In an aspect, a pharmaceutical composition comprises a liquid culture. In another aspect, a pharmaceutical composition is homogenized, lyophilized, pulverized and powdered. It can then be infused, dissolved such as in saline, as an enema. Alternatively, the powder can be encapsulated as enteric-coated and/or acid-resistant delayed release capsules for oral administration. In an aspect, the powder can be double encapsulated with acid-resistant/delayed release capsules for oral administration. These capsules can take the form of enteric-coated and/or acid-resistant delayed release microcapsules. A powder can be provided in a palatable form for reconstitution for drinking or for reconstitution as a food additive. In a further aspect, a food is yogurt. In one aspect, a powder can be reconstituted to be infused via nasoduodenal infusion.

In another aspect, a pharmaceutical composition administered herein is in a liquid, frozen, freeze-dried, spray-dried, foam-dried, lyophilized, or powder form. In a further aspect, a pharmaceutical composition administered herein is formulated as a delayed or gradual enteric release form. In another aspect, a pharmaceutical composition administered herein comprises an excipient, a saline, a buffer, a buffering agent, or a fluid-glucose-cellobiose agar (RGCA) media. In another aspect, a pharmaceutical composition administered herein comprises a cryoprotectant. In one aspect, a cryoprotectant comprises polyethylene glycol, skim milk, erythritol, arabitol, sorbitol, glucose, fructose, alanine, glycine, proline, sucrose, lactose, ribose, trehalose, dimethyl sulfoxide (DMSO), glycerol, or a combination thereof.

In various aspects, provided herein are modified-release formulations comprising a bacterial mixture (e.g., comprising a preparation of uncultured fecal bacteria), wherein the formulation releases a substantial amount of the bacterial mixture (and optionally additional therapeutic agents) into one or more regions of the GI tract. For example, the formulation can release at least about 60% of the bacterial isolates after the stomach and into one or more regions of the GI tract.

In various aspects, the modified-release formulation can release at least 60% of the bacterial mixture (and optionally additional therapeutic agents) after the stomach into one or more regions of the intestine. For example, the modified-release formulation can release at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the bacterial mixture (and optionally additional therapeutic agents) in the intestines.

In various aspects, the modified-release formulation can release at least 60% of the bacterial mixture (and optionally additional therapeutic agents) in the small intestine. For example, the modified-release formulation can release at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the bacterial mixture (and optionally additional therapeutic agents) in the small intestine (e.g., one or more of duodenum, jejunum, ileum, and ileocecal junction).

In various aspects, the modified-release formulation can release at least 60% of the bacterial mixture (and optionally additional therapeutic agents) in the large intestine. For example, the modified-release formulation can release at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the bacterial isolates (and/or additional therapeutic agents) in the large intestine (e.g., one or more of cecum, ascending, transverse, descending or sigmoid portions of the colon, and rectum).

In some aspects, the pharmaceutical composition is formulated for release in the stomach. In other aspects, the pharmaceutical composition is formulated so as to not substantially release the bacterial mixture in the stomach.

In certain aspects, the modified-release formulation releases the bacterial mixture (and optionally additional therapeutic agents) at a specific pH. For example, in some aspects, the modified-release formulation is substantially stable in an acidic environment and substantially unstable (e.g., dissolves rapidly or is physically unstable) in a near neutral to alkaline environment. In some aspects, stability is indicative of not substantially releasing while instability is indicative of substantially releasing. For example, in some aspects, the modified-release formulation is substantially stable at a pH of about 7.0 or less, or about 6.5 or less, or about 6.0 or less, or about 5.5 or less, or about 5.0 or less, or about 4.5 or less, or about 4.0 or less, or about 3.5 or less, or about 3.0 or less, or about 2.5 or less, or about 2.0 or less, or about 1.5 or less, or about 1.0 or less. In some aspects, the present formulations are stable in lower pH areas and therefore do not substantially release in, for example, the stomach. In some aspects, modified-release formulation is substantially stable at a pH of about 1 to about 4 or lower and substantially unstable at pH values that are greater. In these aspects, the modified-release formulation does not substantially release in the stomach. In these aspects, the modified-release formulation substantially releases in the small intestine (e.g. one or more of the duodenum, jejunum, and ileum) and/or large intestine (e.g. one or more of the cecum, ascending colon, transverse colon, descending colon, and sigmoid colon). In some aspects, modified-release formulation is substantially stable at a pH of about 4 to about 5 or lower and consequentially is substantially unstable at pH values that are greater and therefore is not substantially released in the stomach and/or small intestine (e.g. one or more of the duodenum, jejunum, and ileum). In these aspects, the modified-release formulation substantially releases in the large intestine (e.g. one or more of the cecum, ascending colon, transverse colon, descending colon, and sigmoid colon). In various aspects, the pH values recited herein can be adjusted as known in the art to account for the state of the subject, e.g. whether in a fasting or postprandial state.

In some aspects, the modified-release formulation is substantially stable in gastric fluid and substantially unstable in intestinal fluid and, accordingly, is substantially released in the small intestine (e.g. one or more of the duodenum, jejunum, and ileum) and/or large intestine (e.g. one or more of the cecum, ascending colon, transverse colon, descending colon, and sigmoid colon).

In some aspects, the modified-release formulation is stable in gastric fluid or stable in acidic environments. These modified-release formulations release about 30% or less by weight of the pharmaceutical composition (e.g., comprising a bacterial mixture) in the modified-release formulation in gastric fluid with a pH of about 4 to about 5 or less, or simulated gastric fluid with a pH of about 4 to about 5 or less, in about 15, or about 30, or about 45, or about 60, or about 90 minutes. Modified-release formulations of can release from about 0% to about 30%, from about 0% to about 25%, from about 0% to about 20%, from about 0% to about 15%, from about 0% to about 10%, about 5% to about 30%, from about 5% to about 25%, from about 5% to about 20%, from about 5% to about 15%, from about 5% to about 10% by weight of the composition in the modified-release formulation in gastric fluid with a pH of 4-5, or less or simulated gastric fluid with a pH of 4-5 or less, in about 15, or about 30, or about 45, or about 60, or about 90 minutes. Modified-release formulations can release about 1%, about 2%, about 3%, about 4%, about 5%, about 6%, about 7%, about 8%, about 9%, or about 10% by weight of the total composition in the modified-release formulation in gastric fluid with a pH of 5 or less, or simulated gastric fluid with a pH of 5 or less, in about 15, or about 30, or about 45, or about 60, or about 90 minutes.

In some aspects, the modified-release formulation is unstable in intestinal fluid. These modified-release formulations release about 70% or more by weight of the bacterial mixture and/or additional therapeutic agent in the modified-release formulation in intestinal fluid or simulated intestinal fluid in about 15, or about 30, or about 45, or about 60, or about 90 minutes. In some aspects, the modified-release formulation is unstable in near neutral to alkaline environments. These modified-release formulations release about 70% or more by weight of the bacterial mixture and/or additional therapeutic agent in the modified-release formulation in intestinal fluid with a pH of about 4-5 or greater, or simulated intestinal fluid with a pH of about 4-5 or greater, in about 15, or about 30, or about 45, or about 60, or about 90 minutes. A modified-release formulation that is unstable in near neutral or alkaline environments can release 70% or more by weight of the pharmaceutical composition (e.g., comprising a microbial cocktail) in the modified-release formulation in a fluid having a pH greater than about 5 (e.g., a fluid having a pH of from about 5 to about 14, from about 6 to about 14, from about 7 to about 14, from about 8 to about 14, from about 9 to about 14, from about 10 to about 14, or from about 11 to about 14) in from about 5 minutes to about 90 minutes, or from about 10 minutes to about 90 minutes, or from about 15 minutes to about 90 minutes, or from about 20 minutes to about 90 minutes, or from about 25 minutes to about 90 minutes, or from about 30 minutes to about 90 minutes, or from about 5 minutes to about 60 minutes, or from about 10 minutes to about 60 minutes, or from about 15 minutes to about 60 minutes, or from about 20 minutes to about 60 minutes, or from about 25 minutes to about 90 minutes, or from about 30 minutes to about 60 minutes.

Examples of simulated gastric fluid and simulated intestinal fluid include, but are not limited to, those disclosed in the 2005 Pharmacopeia 23NF/28USP in Test Solutions at page 2858 and/or other simulated gastric fluids and simulated intestinal fluids known to those of skill in the art, for example, simulated gastric fluid and/or intestinal fluid prepared without enzymes.

In various aspects, the modified-release formulation can be substantially stable in chyme. For example, there is, in some aspects, a loss of less about 50% or about 40%, or about 30%, or about 20%, or about 10% of the activity or viability of the bacteria in the bacterial mixture in about 10, or 9, or 8, or 7, or 6, or 5, or 4, or 3, or 2, or 1 hour from administration.

In various aspects, the modified-release formulations can be designed for immediate release (e.g. upon ingestion). In various aspects, the modified-release formulations can have sustained-release profiles, i.e. slow release of the active ingredient(s) in the body (e.g., GI tract) over an extended period of time. In various aspects, the modified-release formulations can have a delayed-release profile, i.e. not immediately release the active ingredient(s) upon ingestion; rather, postponement of the release of the active ingredient(s) until the composition is lower in the GI tract; for example, for release in the small intestine (e.g., one or more of duodenum, jejunum, ileum) or the large intestine (e.g., one or more of cecum, ascending, transverse, descending or sigmoid portions of the colon, and rectum). For example, a composition can be enteric coated to delay release of the active ingredient(s) until it reaches the small intestine or large intestine.

In various aspects, the modified-release formulations can utilize one or more modified-release coatings such as delayed-release coatings to provide for effective, delayed yet substantial delivery of the bacterial mixture to the GI tract together with, optionally, additional therapeutic agents.

In an aspect, the delayed-release coating includes an enteric agent that is substantially stable in acidic environments and substantially unstable in near neutral to alkaline environments. In an aspect, the delayed-release coating contains an enteric agent that is substantially stable in gastric fluid. The enteric agent can be selected from, for example, solutions or dispersions of methacrylic acid copolymers, cellulose acetate phthalate, hydroxypropylmethyl cellulose phthalate, polyvinyl acetate phthalate, carboxymethylethylcellulose, and EUDRAGIT®-type polymer (poly(methacrylic acid, methylmethacrylate), hydroxypropyl methylcellulose acetate succinate, cellulose acetate trimellitate, shellac or other suitable enteric coating polymers. The EUDRAGIT®-type polymers include, for example, EUDRAGIT® FS 30D, L 30 D-55, L 100-55, L 100, L 12,5, L 12,5 P, RL 30 D, RL PO, RL 100, RL 12,5, RS 30 D, RS PO, RS 100, RS 12,5, NE 30 D, NE 40 D, NM 30 D, S 100, S 12,5, and S 12,5 P. Similar polymers include Kollicoat® MAE 30 DP and Kollicoat® MAE 100 P. In some aspects, one or more of EUDRAGIT® FS 30D, L 30 D-55, L 100-55, L 100, L 12,5, L 12,5 P RL 30 D, RL PO, RL 100, RL 12,5, RS 30 D, RS PO, RS 100, RS 12,5, NE 30 D, NE 40 D, NM 30 D, S 100, S 12,5 S 12,5 P, Kollicoat® MAE 30 DP and Kollicoat® MAE 100 P is used. In various aspects, the enteric agent can be a combination of the foregoing solutions or dispersions.

In certain aspects, one or more coating system additives are used with the enteric agent. For example, one or more PlasACRYL™ additives can be used as an anti-tacking agent coating additive. Illustrative PlasACRYL™ additives include, but are not limited to, PlasACRYL™ HTP20 and PlasACRYL™ T20.

In another aspect, the delayed-release coating can degrade as a function of time when in aqueous solution without regard to the pH and/or presence of enzymes in the solution. Such a coating can comprise a water insoluble polymer. Its solubility in aqueous solution is therefore independent of the pH. The term “pH independent” as used herein means that the water permeability of the polymer and its ability to release pharmaceutical ingredients is not a function of pH and/or is only very slightly dependent on pH. Such coatings can be used to prepare, for example, sustained release formulations. Suitable water insoluble polymers include pharmaceutically acceptable nontoxic polymers that are substantially insoluble in aqueous media, e.g., water, independent of the pH of the solution. Suitable polymers include, but are not limited to, cellulose ethers, cellulose esters, or cellulose etheresters, i.e., a cellulose derivative in which some of the hydroxy groups on the cellulose skeleton are substituted with alkyl groups and some are modified with alkanoyl groups. Examples include ethyl cellulose, acetyl cellulose, nitrocellulose, and the like. Other examples of insoluble polymers include, but are not limited to, lacquer, and acrylic and/or methacrylic ester polymers, polymers or copolymers of acrylate or methacrylate having a low quaternary ammonium content, or mixture thereof and the like. Other examples of insoluble polymers include EUDRAGIT RS®, EUDRAGIT RL®, and EUDRAGIT NE®. Insoluble polymers can include polyvinyl esters, polyvinyl acetals, polyacrylic acid esters, butadiene styrene copolymers, and the like. In an aspect, colonic delivery is achieved by use of a slowly eroding wax plug (e.g., various PEGS, including for example, PEG6000).

In a further aspect, the delayed-release coating can be degraded by a microbial enzyme present in the gut flora. In an aspect, the delayed-release coating can be degraded by bacteria present in the small intestine. In another aspect, the delayed-release coating can be degraded by bacteria present in the large intestine.

In various aspects, the modified release formulation can be designed for release in the colon. Various colon-specific delivery approaches can be utilized. For example, the modified release formulation can be formulated using a colon-specific drug delivery system (CODES) as described for example, in Li et al., AAPS Pharm Sci Tech (2002), 3(4): 1-9, the entire contents of which are incorporated herein by reference. Drug release in such a system is triggered by colonic microflora coupled with pH-sensitive polymer coatings. For example, the formulation can be designed as a core tablet with three layers of polymer. The first coating is an acid-soluble polymer (e.g., EU-DRAGIT E), the outer coating is enteric, along with a hydroxypropyl methylcellulose barrier layer interposed in between. In another aspect, colon delivery can be achieved by formulating the pharmaceutical composition (e.g., comprising a microbial cocktail) with specific polymers that degrade in the colon such as, for example, pectin. The pectin can be further gelled or crosslinked with a cation such as a zinc cation. In an aspect, the formulation is in the form of ionically cross-linked pectin beads which are further coated with a polymer (e.g., EUDRAGIT polymer). Additional colon specific formulations include, but are not limited to, pressure-controlled drug delivery systems (prepared with, for example, ethylcellulose) and osmotic controlled drug delivery systems (i.e., ORDSCT).

Formulations for colon specific delivery of the bacterial mixture (and/or additional therapeutic agents), as described herein, can be evaluated using, for example, in vitro dissolution tests. For example, parallel dissolution studies in different buffers can be undertaken to characterize the behavior of the formulations at different pH levels. Alternatively, in vitro enzymatic tests can be carried out. For example, the formulations can be incubated in fermenters containing suitable medium for bacteria, and the amount of drug released at different time intervals is determined. Drug release studies can also be done in buffer medium containing enzymes or rat or guinea pig or rabbit cecal contents and the amount of drug released in a particular time is determined. In a further aspect, in vivo evaluations can be carried out using animal models such as dogs, guinea pigs, rats, and pigs. Further, clinical evaluation of colon specific drug delivery formulations can be evaluated by calculating drug delivery index (DDI) which considers the relative ratio of RCE (relative colonic tissue exposure to the drug) to RSC (relative amount of drug in blood i.e. that is relative systemic exposure to the drug). Higher drug DDI indicates better colon drug delivery. Absorption of drugs from the colon can be monitored by colonoscopy and intubation.

In various aspects, the present formulations provide for substantial uniform delivery of the bacterial mixture (and/or additional therapeutic agent) in the area of release in the GI tract. In an aspect, the present formulations minimize patchy or heterogeneous release of the bacterial mixture.

In various aspects, the present formulations provide for release of multiple doses of one or more bacterial mixtures along the GI tract. For example, the composition and/or formulation can release multiple doses of the same bacterial mixture at different locations along the intestines, at different times, and/or at different pH. Alternatively, the composition and/or formulation can release a dose of different bacterial mixtures at different locations along the intestines, at different times, and/or at a different pH. In an aspect, the pharmaceutical composition comprises a first bacterial mixture comprising one or more bacterial isolates that is released at a first location in the intestine, and a second bacterial mixture comprising a preparation of uncultured fecal bacteria that is released at a second location in the intestine. In an aspect, the first bacterial mixture is released in the ileum, and the second bacterial mixture is released in the colon.

The overall release profile of such a formulation can be adjusted using, for example, multiple particle types or multiple layers. For example, in an aspect, a first bacterial mixture (or first dose of a bacterial mixture) can be formulated for release in, for example, the small intestine (e.g., one or more of duodenum, jejunum, ileum), whereas the second bacterial mixture (or second dose of the bacterial mixture) is formulated for delayed release in, for example, the large intestine (e.g., one or more of cecum, ascending, transverse, descending or sigmoid portions of the colon, and rectum). In another example, the first bacterial mixture (or first dose of a bacterial mixture) can be formulated for release in, for example, the small intestine (e.g., one or more of duodenum, jejunum, ileum), whereas the second bacterial mixture (or second dose of a bacterial mixture) is formulated for delayed release in, for example, another part of the small intestine (e.g., one or more of duodenum, jejunum, ileum). In another aspect, the first bacterial mixture (or first dose of a bacterial mixture) can be formulated for release in, for example, the large intestine (e.g., one or more of cecum, ascending, transverse, descending or sigmoid portions of the colon, and rectum), whereas the second bacterial mixture (or second dose of the bacterial mixture) is formulated for delayed release in, for example, another part of the large intestine (e.g., one or more of cecum, ascending, transverse, descending or sigmoid portions of the colon, and rectum). In various aspects, the composition and/or formulation can release at least one dose, at least two doses, at least three doses, at least four doses, or at least five doses of the bacterial mixture at different locations along the intestines, at different times, and/or at different pH. Likewise, in various aspects, the composition and/or formulation can release at least one bacterial mixture, at least two bacterial mixtures, at least three bacterial mixtures, at least four bacterial mixtures, or at least five bacterial mixtures at different locations along the intestines, at different times, and/or at different pH.

In another aspect, a delayed or gradual enteric release formulation comprises the use of a bilayer tablet or capsule which comprises a first layer comprising a polyalkylene oxide, a polyvinylpyrrolidone, a lubricant, or a mixture thereof, and a second osmotic push layer comprising polyethylene oxide, carboxymethylcellulose, or both. In an aspect, a delayed or gradual enteric release formulation comprises the use of a release-retarding matrix material selected from the group consisting of an acrylic polymer, a cellulose, a wax, a fatty acid, shellac, zein, hydrogenated vegetable oil, hydrogenated castor oil, polyvinylpyrrolidine, a vinyl acetate copolymer, a vinyl alcohol copolymer, polyethylene oxide, an acrylic acid and methacrylic acid copolymer, a methyl methacrylate copolymer, an ethoxyethyl methacrylate polymer, a cyanoethyl methacrylate polymer, an amino-alkyl methacrylate copolymer, a poly(acrylic acid), a poly(methacrylic acid), a methacrylic acid alkylamide copolymer, a poly(methyl methacrylate), a poly(methacrylic acid anhydride), a methyl methacrylate polymer, a polymethacrylate, a poly(methyl methacrylate) copolymer, a polyacrylamide, an aminoalkyl methacrylate copolymer, a glycidyl methacrylate copolymer, a methyl cellulose, an ethylcellulose, a carboxymethyl cellulose, a hydroxypropylmethylcellulose, a hydroxymethyl cellulose, a hydroxyethyl cellulose, a hydroxypropyl cellulose, a crosslinked sodium carboxymethylcellulose, a crosslinked hydroxypropylcellulose, a natural wax, a synthetic wax, a fatty alcohol, a fatty acid, a fatty acid ester, a fatty acid glyceride, a hydrogenated fat, a hydrocarbon wax, stearic acid, stearyl alcohol, beeswax, glycowax, castor wax, carnauba wax, a polylactic acid, polyglycolic acid, a copolymer of lactic and glycolic acid, carboxymethyl starch, potassium methacrylate/divinylbenzene copolymer, crosslinked polyvinylpyrrolidone, poly inylalcohols, polyvinylalcohol copolymers, polyethylene glycols, non-crosslinked polyvinylpyrrolidone, polyvinylacetates, polyvinylacetate copolymers, or any combination thereof. In an aspect, a delayed or gradual enteric release formulation comprises the use of a microenvironment pH modifier.

It will be understood that a pharmaceutical composition described herein can comprise multiple distinct bacterial mixtures, for example to achieve different delivery location profiles for each bacterial mixture. In an aspect, a pharmaceutical composition comprises at least two bacterial mixtures, such that a first bacterial mixture comprises one or more bacterial isolates and a second bacterial mixture comprises a preparation of uncultured fecal bacteria. In an aspect, the second bacterial mixture further comprises one or more bacterial isolates that are different than the bacterial isolate(s) in the first bacterial mixture. Alternatively, the second bacterial mixture can consist essentially of the preparation of uncultured fecal bacteria. In another aspect, the first bacterial mixture can comprise only one bacterial isolate. A pharmaceutical composition can comprise any number of bacterial mixtures, for example one, two, three, four, five, six, seven, eight, nine, ten, or more than ten bacterial mixtures that each contain a different bacterial isolate, a different combination of bacterial isolates, a preparation of uncultured fecal bacteria, or a different combination of uncultured fecal bacteria with one or more bacterial isolates.

In an aspect, a pharmaceutical composition can be a drench. In one aspect, a drench is prepared by choosing a saline-suspended form of a pharmaceutical composition. A water-soluble form of one ingredient can be used in conjunction with a water-insoluble form of the other by preparing a suspension of one with an aqueous solution of the other. Water-insoluble forms of either active ingredient may be prepared as a suspension or in some physiologically acceptable solvent such as polyethylene glycol. Suspensions of water-insoluble forms of either active ingredient can be prepared in oils such as peanut, corn, sesame oil or the like; in a glycol such as propylene glycol or a polyethylene glycol; or in water depending on the solubility of a particular active ingredient. Suitable physiologically acceptable adjuvants may be necessary in order to keep the active ingredients suspended. Adjuvants can include and be chosen from among the thickeners, such as carboxymethylcellulose, polyvinyl pyrrolidone, gelatin and the alginates. Surfactants generally will serve to suspend the active ingredients, particularly the fat-soluble propionate-enhancing compounds. Most useful for making suspensions in liquid nonsolvents are alkylphenol polyethylene oxide adducts, naphthalenesulfonates, alkylbenzene-sulfonates, and the polyoxyethylene sorbitan esters. In addition many substances, which affect the hydrophilicity, density and surface tension of the liquid, can assist in making suspensions in individual cases. For example, silicone antifoams, glycols, sorbitol, and sugars can be useful suspending agents.

In some aspects, one or more bacterial isolates described herein are in the form of live, vegetative cells. In some aspects, one or more bacterial isolates described herein are in the form of spores. In some aspects, one or more bacterial isolates described herein are lyophilized. By way of non-limiting example, lyophilization can be via methods known in the art, including those described in U.S. Pat. No. 7,799,328, the contents of which are hereby incorporated by reference in their entirety. In some aspects, lyophilized bacterial mixtures described herein are placed in an enterically coated soft gel or capsule.

In various aspects, formulations can take the form of those described in one or more of U.S. Pat. Nos. 8,535,713 and 8,911,777 and US Patent Publication Nos. 20120141585, 20120141531, 2006/001896, 2007/0292523, 2008/0020018, 2008/0113031, 2010/0203120, 2010/0255087, 2010/0297221, 2011/0052645, 2013/0243873, 2013/0330411, 2014/0017313, and 2014/0234418, the contents of which are hereby incorporated by reference in their entirety.

In various aspects, formulations can take the form of those as described in International Patent Publication No. WO 2008/135090, the contents of which are hereby incorporated by reference in their entirety.

In various aspects, formulations can take the form of those described in one or more of U.S. Pat. Nos. 4,196,564; 4,196,565; 4,247,006; 4,250,997; 4,268,265; 5,317,849; 6,572,892; 7,712,634; 8,074,835; 8,398,912; 8,440,224; 8,557,294; 8,646,591; 8,739,812; 8,810,259; 8,852,631; and 8,911,788 and US Patent Publication Nos. 2014/0302132; 2014/0227357; 20140088202; 20130287842; 2013/0295188; 2013/0307962; and 20130184290, the contents of which are hereby incorporated by reference in their entirety.

It will be appreciated that the dose of a pharmaceutical composition or the bacterial cells therein (e.g., a bacterial mixture comprising one or more bacterial isolates and/or a preparation of uncultured fecal bacteria) will vary according to, for example, the particular dosage form, the mode of administration to a subject, the identity of a bacterial isolate, if any, in the composition, the number of bacterial isolates, if any, in the composition. These factors, as well as variables that may modify the activity of the bacteria in a bacterial mixture (e.g., subject body weight, sex and diet, time of administration, route of administration, rate of excretion, condition of the subject, drug combinations, genetic disposition and reaction sensitivities) can be taken into account by those skilled in the art to generate an effective dose or dosage regime for treatment or prevention of at least one symptom of a disorder described herein. Administration can be carried out continuously or in one or more discrete doses within the maximum tolerated dose. Optimal administration rates for a given set of conditions can be ascertained by those skilled in the art using conventional dosage administration tests.

In various aspects, the dose of the pharmaceutical composition or the bacterial cells therein (e.g., a bacterial mixture comprising one or more bacterial isolates and/or a preparation of uncultured fecal bacteria) is effective to modulate a patient's microbiome to favor an ecological balance, so as to treat or prevent one or more symptoms of a disorder associated with a gut dysbiosis.

In one aspect, a pharmaceutically active or therapeutically effective dose of a bacterial isolate administered to a subject (i.e., in single or multiple administrations) to treat at least one symptom of a disorder comprises at least 10⁵, at least 10⁶, at least 10⁷, at least 10⁸, at least 10⁹, at least 10¹⁰, at least 10¹¹, at least 10¹², at least 10¹³, at least 10¹⁴, or at least 10¹⁵ CFUs of the bacterial isolate. In another aspect, a pharmaceutically active or therapeutically effective dose of a bacterial isolate administered to a subject (i.e., in single or multiple administrations) to treat at least one symptom of a disorder comprises at most 10⁵, at most 10⁶, at most 10⁷, at most 10⁸, at most 10⁹, at most 10¹⁰, at most 10¹¹, at most 10¹², at most 10¹³, at most 10¹⁴, or at most 10¹⁵ CFUs of the bacterial isolate. In a further aspect, a pharmacologically active or therapeutically effective dose of a bacterial isolate administered to a subject (i.e., in single or multiple administrations) to treat at least one symptom of a disorder is selected from the group consisting of: from 10⁸ CFUs to 10¹⁴ CFUs, from 10⁹ CFUs to 10¹³ CFUs, from 10¹⁰ CFUs to 10¹² CFUs, from 10¹⁰ CFUs to 10¹¹ CFUs, from 10⁹ CFUs to 10¹⁴ CFUs, from 10⁹ CFUs to 10¹² CFUs, from 10⁹ CFUs to 10¹¹ CFUs, from 10⁹ CFUs to 10¹⁰ CFUs, from 10¹⁰ CFUs to 10¹⁴ CFUs, from 10¹⁰ CFUs to 10¹³ CFUs, from 10¹¹ CFUs to 10¹⁴ CFUs, from 10¹¹ CFUs to 10¹³ CFUs, from 10¹² CFUs to 10¹⁴ CFUs, and from 10¹³ CFUs to 10¹⁴ CFUs of the bacterial isolate.

In an aspect, a pharmaceutical composition comprises one or more bacterial isolates, with each bacterial isolate present in each unit dose at one of the foregoing pharmaceutically active or therapeutically effective doses in a unit weight of about 0.2, 0.4, 0.6, 0.8 or 1.0 gram, or a unit volume of about 0.2, 0.4, 0.6, 0.8 or 1.0 milliliter.

In one aspect, a pharmaceutically active or therapeutically effective dose of a bacterial isolate administered to a subject (i.e., in single or multiple administrations) to treat at least one symptom of a disorder comprises at least 10⁵, at least 10⁶, at least 10⁷, at least 10⁸, at least 10⁹, at least 10¹⁰, at least 10¹¹, at least 10¹², at least 10¹³, at least 10¹⁴, or at least 10¹⁵ cells or spores of the bacterial isolate. In another aspect, a pharmaceutically active or therapeutically effective dose of a bacterial isolate administered to a subject i.e. in single or multiple administrations) to treat at least one symptom of a disorder comprises at most 10⁵, at most 10⁶, at most 10⁷, at most 10⁸, at most 10⁹, at most 10¹⁰, at most 10¹¹, at most 10¹², at most 10¹³, at most 10¹⁴, or at most 10¹⁵ total cells or spores of the bacterial isolate. In a further aspect, a pharmacologically active or therapeutically effective dose of a bacterial isolate administered to a subject (i.e., in single or multiple administrations) to treat at least one symptom of a disorder is selected from the group consisting of: from 10⁸ to 10¹⁴, from 10⁹ to 10¹³, from 10¹⁰ to 10¹², from 10¹⁰ to 10¹¹, from 10⁹ to 10¹⁴, from 10⁹ to 10¹², from 10⁹ to 10¹¹, from 10⁹ to 10¹⁰, from 10¹⁰ to 10¹⁴, from 10¹⁰ to 10¹³, from 10¹¹ to 10¹⁴, from 10¹¹ to 10¹³, from 10¹² to 10¹⁴, and from 10¹³ to 10¹⁴ cells or spores of the bacterial isolate.

In an aspect, the pharmaceutically active or therapeutically effective dose cell count of a bacterial isolate is directed to live cells. In one aspect, a pharmaceutical composition comprises one or more bacterial isolates, with each bacterial isolates present in each dosage unit at one of the foregoing pharmaceutically active or therapeutically effective doses in a unit weight of about 0.2, 0.4, 0.6, 0.8 or 1.0 gram, or a unit volume of about 0.2, 0.4, 0.6, 0.8 or 1.0 milliliter.

In an aspect, a pharmaceutical composition described herein is in the form of a capsule, and each capsule comprises at least 10⁵, at least 10⁶, at least 10⁷, at least 10⁸, at least 10⁹, at least 10¹⁰, at least 10¹¹, at least 10¹², at least 10¹³, at least 10¹⁴, or at least 10¹⁵ cells or spores of a bacterial isolate. In an aspect, a pharmaceutical composition described herein is in the form of a capsule, and each capsule comprises from 10⁸ to 10¹⁴, from 10⁹ to 10¹³, from 10¹⁰ to 10¹², from 10¹⁰ to 10¹¹, from 10⁹ to 10¹⁴, from 10⁹ to 10¹², from 10⁹ to 10¹¹, from 10⁹ to 10¹⁰, from 10¹⁰ to 10¹⁴, from 10¹⁰ to 10¹³, from 10¹¹ to 10¹⁴, from 10¹¹ to 10¹³, from 10¹² to 10¹⁴, or from 10¹³ to 10¹⁴ cells or spores of a bacterial isolate.

In one aspect, a pharmaceutically active or therapeutically effective dose of a preparation of uncultured fecal bacteria administered to a subject (i.e., in single or multiple administrations) to treat at least one symptom of a disorder (e.g., associated with a gut dysbiosis) comprises at least 10⁵, at least 10⁶, at least 10⁷, at least 10⁸, at least 10⁹, at least 10¹⁰, at least 10¹¹, at least 10¹², at least 10¹³, at least 10¹⁴, or at least 10¹⁵ CFUs of the preparation of uncultured fecal bacteria. In another aspect, a pharmaceutically active or therapeutically effective dose of a preparation of uncultured fecal bacteria administered to a subject (i.e., in single or multiple administrations) to treat at least one symptom of a disorder (e.g., associated with a gut dysbiosis) comprises at most 10⁵, at most 10⁶, at most 10⁷, at most 10⁸, at most 10⁹, at most 10¹⁰, at most 10¹¹, at most 10¹², at most 10¹³, at most 10¹⁴, or at most 10¹⁵ CFUs of the preparation of uncultured fecal bacteria. In a further aspect, a pharmacologically active or therapeutically effective dose of a preparation of uncultured fecal bacteria administered to a subject (i.e., in single or multiple administrations) to treat at least one symptom of a disorder (e.g., associated with a gut dysbiosis) is selected from the group consisting of: from 10⁸ CFUs to 10¹⁴ CFUs, from 10⁹ CFUs to 10¹³ CFUs, from 10¹⁰ CFUs to 10¹² CFUs, from 10¹⁰ CFUs to 10¹¹ CFUs, from 10⁹ CFUs to 10¹⁴ CFUs, from 10⁹ CFUs to 10¹² CFUs, from 10⁹ CFUs to 10¹¹ CFUs, from 10⁹ CFUs to 10¹⁰ CFUs, from 10¹⁰ CFUs to 10¹⁴ CFUs, from 10¹⁰ CFUs to 10¹³ CFUs, from 10¹¹ CFUs to 10¹⁴ CFUs, from 10¹¹ CFUs to 10¹³ CFUs, from 10¹² CFUs to 10¹⁴ CFUs, and from 10¹³ CFUs to 10¹⁴ CFUs of the preparation of uncultured fecal bacteria.

In an aspect, uncultured fecal bacteria are present in each unit dose of a pharmaceutical composition at one of the foregoing pharmaceutically active or therapeutically effective doses in a unit weight of about 0.2, 0.4, 0.6, 0.8 or 1.0 gram, or a unit volume of about 0.2, 0.4, 0.6, 0.8 or 1.0 milliliter.

In one aspect, a pharmaceutically active or therapeutically effective dose of a preparation of uncultured fecal bacteria administered to a subject (i.e., in single or multiple administrations) to treat at least one symptom of a disorder (e.g., associated with a gut dysbiosis) comprises at least 10⁵, at least 10⁶, at least 10⁷, at least 10⁸, at least 10⁹, at least 10¹⁰, at least 10¹¹, at least 10¹², at least 10¹³, at least 10¹⁴, or at least 10¹⁵ cells or spores of the preparation of uncultured fecal bacteria. In another aspect, a pharmaceutically active or therapeutically effective dose of a preparation of uncultured fecal bacteria administered to a subject i.e. in single or multiple administrations) to treat at least one symptom of a disorder (e.g., associated with a gut dysbiosis) comprises at most 10⁵, at most 10⁶, at most 10⁷, at most 10⁸, at most 10⁹, at most 10¹⁰, at most 10¹¹, at most 10¹², at most 10¹³, at most 10¹⁴, or at most 10¹⁵ total cells or spores of the preparation of uncultured fecal bacteria. In a further aspect, a pharmacologically active or therapeutically effective dose of a preparation of uncultured fecal bacteria administered to a subject (i.e., in single or multiple administrations) to treat at least one symptom of a disorder (e.g., associated with a gut dysbiosis) is selected from the group consisting of: from 10⁸ to 10¹⁴, from 10⁹ to 10¹³, from 10¹⁰ to 10¹², from 10¹⁰ to 10¹¹, from 10⁹ to 10¹⁴, from 10⁹ to 10¹², from 10⁹ to 10¹¹, from 10⁹ to 10¹⁰, from 10¹⁰ to 10¹⁴, from 10¹⁰ to 10¹³, from 10¹¹ to 10¹⁴, from 10¹¹ to 10¹³, from 10¹² to 10¹⁴, and from 10¹³ to 10¹⁴ cells or spores of the preparation of uncultured fecal bacteria.

In an aspect, the pharmaceutically active or therapeutically effective dose cell count of a preparation of uncultured fecal bacteria is directed to live cells. In one aspect, a preparation of uncultured fecal bacteria is present in each unit dose of a pharmaceutical composition at one of the foregoing pharmaceutically active or therapeutically effective doses in a unit weight of about 0.2, 0.4, 0.6, 0.8 or 1.0 gram, or a unit volume of about 0.2, 0.4, 0.6, 0.8 or 1.0 milliliter.

In an aspect, a pharmaceutical composition described herein is in the form of a capsule, and each capsule comprises at least 10⁵, at least 10⁶, at least 10⁷, at least 10⁸, at least 10⁹, at least 10¹⁰, at least 10¹¹, at least 10¹², at least 10¹³, at least 10¹⁴, or at least 10¹⁵ cells or spores of a preparation of uncultured fecal bacteria. In an aspect, a pharmaceutical composition described herein is in the form of a capsule, and each capsule comprises from 10⁸ to 10¹⁴, from 10⁹ to 10¹³, from 10¹⁰ to 10¹², from 10¹⁰ to 10¹¹, from 10⁹ to 10¹⁴, from 10⁹ to 10¹², from 10⁹ to 10¹¹, from 10⁹ to 10¹⁰, from 10¹⁰ to 10¹⁴, from 10¹⁰ to 10¹³, from 10¹¹ to 10¹⁴, from 10¹¹ to 10¹³, from 10¹² to 10¹⁴, or from 10¹³ to 10¹⁴ cells or spores of a preparation of uncultured fecal bacteria.

A subject can be administered one or more bacterial isolates combined with a preparation of uncultured fecal bacteria for treatment of one or more symptoms of a disorder. In such cases, the bacterial isolate(s) and preparation of uncultured fecal bacteria can be administered to the subject together in the same pharmaceutical composition, or in separate compositions. Further, a pharmaceutical composition (e.g., comprising one or more bacterial isolates, a preparation of uncultured fecal bacteria, or both) can be administered to the subject in a single unit dose or multiple unit doses, for example as part of a dosage regime. In an aspect, the dosage of the preparation of uncultured fecal bacteria (e.g. measured by CFU or cell/spore count) administered to a subject is greater than the dosage of the bacterial isolate. Alternatively, the dosage of the preparation of uncultured fecal bacteria (e.g. measured by CFU or cell/spore count) administered to the subject can be less than the dosage of the bacterial isolate. In another aspect, the dosage of the preparation of uncultured fecal bacteria (e.g. measured by CFU or cell/spore count) can be about the same as the dosage of the bacterial isolate. For example, in an aspect a subject can be administered a bacterial isolate at a dosage of about 10¹⁰ cells and a preparation of uncultured fecal bacteria at a dosage of about 10¹⁰ cells to treat or prevent one or more symptoms of a disorder described herein.

In an aspect, the number of cells of a bacterial isolate administered to a subject to treat one or more symptoms of a disorder described herein is about the same or greater than the total number of cells of a preparation of uncultured fecal bacteria administered to the subject. Alternatively, the number of cells of a bacterial isolate administered to a subject to treat one or more symptoms of a disorder can be about the same or less than the total number of cells of a preparation of uncultured fecal bacteria administered to the subject.

In an aspect, a pharmaceutical composition comprises a bacterial mixture that comprises multiple bacterial isolates. In another aspect, at least two bacterial isolates are present at about the same amount or dosage (e.g., about the same number of viable cells or spores, or about the same CFUs). In another aspect, at least three bacterial isolates, at least four bacterial isolates, at least five bacterial isolates, at least six bacterial isolates, at least seven bacterial isolates, at least eight bacterial isolates, at least nine bacterial isolates, at least ten bacterial isolates, or more than ten bacterial isolates are present in the pharmaceutical composition at about the same amount or dosage (e.g., about the same number of viable cells or spores, or about the same CFUs). In another aspect, all of the bacterial isolates in a bacterial mixture are present in about the same amounts.

In an aspect, a pharmaceutical composition comprises a bacterial mixture comprising multiple bacterial isolates, and at least two of the multiple bacterial isolates are present at different amounts or dosages (e.g., different numbers of viable cells or spores, or different CFUs). In another aspect, at least three, at least four, at least five, at least six, at least seven, at least eight, at least nine, at least ten, or more than ten bacterial isolates are present in the bacterial mixture at different amounts or dosages.

A pharmaceutical composition can comprise a bacterial mixture comprising multiple bacterial isolates in combination with a preparation of uncultured fecal bacteria. In an aspect, each bacterial isolate is present in the composition at an amount or dosage that is greater than the amount or dosage of the preparation of uncultured fecal bacteria (e.g., measured as numbers of viable cells or spores, or CFUs). In another aspect, each bacterial isolate is present in the composition at an amount or dosage that is less than the amount or dosage of the preparation of uncultured fecal bacteria (e.g., measured as numbers of viable cells or spores, or CFUs). In another aspect, at least one bacterial isolate is present in the composition at an amount or dosage that is greater than the amount or dosage of the preparation of uncultured fecal bacteria, and at least one bacterial isolate is present in the composition at an amount or dosage that is less than the amount or dosage of the preparation of uncultured fecal bacteria (e.g., measured as numbers of viable cells or spores, or CFUs).

In an aspect, a pharmaceutical composition comprises one or more bacterial isolates at an amount or dosage which is at or above the minimum amount or dosage of the bacterial isolate required to be administered to a subject for engraftment of the bacterial isolate to occur in the intestine of the subject. For example, a minimum dosage of the bacterial isolate required for engraftment of the bacterial isolate into the intestine of the subject can be at least 10⁶ cells, at least 10⁷ cells, at least 10⁸ cells, at least 10⁹ cells, at least 10¹⁰ cells, at least 10¹¹ cells, or at least 10¹² cells. In an aspect a first and second bacterial isolate of a microbial cocktail engraft in the intestine of a subject at different minimal dosages or amounts, and a dosage or amount of each of the first and second bacterial isolate in the microbial cocktail varies corresponding to the respective minimal dosage or amount required for engraftment of the respective bacterial isolate.

Individual doses of the pharmaceutical composition (e.g., comprising a bacterial mixture) can be administered in unit dosage forms (e.g., tablets or capsules) containing, for example, from about 0.01 mg to about 5,000 mg, from about 0.01 mg to about 4,000 mg, from about 0.01 mg to about 3,000 mg, from about 0.01 mg to about 2,000 mg, from about 0.01 mg to about 1,000 mg, from about 0.01 mg to about 950 mg, from about 0.01 mg to about 900 mg, from about 0.01 mg to about 850 mg, from about 0.01 mg to about 800 mg, from about 0.01 mg to about 750 mg, from about 0.01 mg to about 700 mg, from about 0.01 mg to about 650 mg, from about 0.01 mg to about 600 mg, from about 0.01 mg to about 550 mg, from about 0.01 mg to about 500 mg, from about 0.01 mg to about 450 mg, from about 0.01 mg to about 400 mg, from about 0.01 mg to about 350 mg, from about 0.01 mg to about 300 mg, from about 0.01 mg to about 250 mg, from about 0.01 mg to about 200 mg, from about 0.01 mg to about 150 mg, from about 0.01 mg to about 100 mg, from about 0.1 mg to about 90 mg, from about 0.1 mg to about 80 mg, from about 0.1 mg to about 70 mg, from about 0.1 mg to about 60 mg, from about 0.1 mg to about 50 mg, from about 0.1 mg to about 40 mg, from about 0.1 mg to about 30 mg, from about 0.1 mg to about 20 mg, from about 0.1 mg to about 10 mg, from about 0.1 mg to about 5 mg, from about 0.1 mg to about 3 mg, from about 0.1 mg to about 1 mg of the active ingredient per unit dosage form, or from about 5 mg to about 80 mg per unit dosage form. For example, a unit dosage form can include about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1,000 mg, about 2,000 mg, about 3,000 mg, about 4,000 mg, or about 5,000 mg of the active ingredient, inclusive of all values and ranges therebetween.

In an aspect, the pharmaceutical composition (e.g., comprising a bacterial mixture) is administered at an amount of from about 0.01 mg to about 100 mg daily, an amount of from about 0.01 mg to about 5,000 mg daily, about 0.01 mg to about 4,000 mg daily, about 0.01 mg to about 3,000 mg daily, about 0.01 mg to about 2,000 mg daily, about 0.01 mg to about 1,000 mg daily, from about 0.01 mg to about 950 mg daily, from about 0.01 mg to about 900 mg daily, from about 0.01 mg to about 850 mg daily, from about 0.01 mg to about 800 mg daily, from about 0.01 mg to about 750 mg daily, from about 0.01 mg to about 700 mg daily, from about 0.01 mg to about 650 mg daily, from about 0.01 mg to about 600 mg daily, from about 0.01 mg to about 550 mg daily, from about 0.01 mg to about 500 mg daily, from about 0.01 mg to about 450 mg daily, from about 0.01 mg to about 400 mg daily, from about 0.01 mg to about 350 mg daily, from about 0.01 mg to about 300 mg daily, from about 0.01 mg to about 250 mg daily, from about 0.01 mg to about 200 mg daily, from about 0.01 mg to about 150 mg daily, from about 0.1 mg to about 100 mg daily, from about 0.1 mg to about 95 mg daily, from about 0.1 mg to about 90 mg daily, from about 0.1 mg to about 85 mg daily, from about 0.1 mg to about 80 mg daily, from about 0.1 mg to about 75 mg daily, from about 0.1 mg to about 70 mg daily, from about 0.1 mg to about 65 mg daily, from about 0.1 mg to about 60 mg daily, from about 0.1 mg to about 55 mg daily, from about 0.1 mg to about 50 mg daily, from about 0.1 mg to about 45 mg daily, from about 0.1 mg to about 40 mg daily, from about 0.1 mg to about 35 mg daily, from about 0.1 mg to about 30 mg daily, from about 0.1 mg to about 25 mg daily, from about 0.1 mg to about 20 mg daily, from about 0.1 mg to about 15 mg daily, from about 0.1 mg to about 10 mg daily, from about 0.1 mg to about 5 mg daily, from about 0.1 mg to about 3 mg daily, from about 0.1 mg to about 1 mg daily, or from about 5 mg to about 80 mg daily. In various aspects, the bacterial mixture (and/or additional therapeutic agents) is administered at a daily dose of about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1,000 mg, about 2,000 mg, about 3,000 mg, about 4,000 mg, or about 5,000 mg inclusive of all values and ranges therebetween.

In some aspects, a suitable dosage of the pharmaceutical composition (e.g., comprising a bacterial mixture) is in a range of about 0.01 mg/kg to about 100 mg/kg of body weight of the subject, for example, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, 1.9 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg body weight, about 20 mg/kg body weight, about 30 mg/kg body weight, about 40 mg/kg body weight, about 50 mg/kg body weight, about 60 mg/kg body weight, about 70 mg/kg body weight, about 80 mg/kg body weight, about 90 mg/kg body weight, or about 100 mg/kg body weight, inclusive of all values and ranges therebetween. In other aspects, a suitable dosage of the composition in a range of about 0.01 mg/kg to about 100 mg/kg of body weight, in a range of about 0.01 mg/kg to about 90 mg/kg of body weight, in a range of about 0.01 mg/kg to about 80 mg/kg of body weight, in a range of about 0.01 mg/kg to about 70 mg/kg of body weight, in a range of about 0.01 mg/kg to about 60 mg/kg of body weight, in a range of about 0.01 mg/kg to about 50 mg/kg of body weight, in a range of about 0.01 mg/kg to about 40 mg/kg of body weight, in a range of about 0.01 mg/kg to about 30 mg/kg of body weight, in a range of about 0.01 mg/kg to about 20 mg/kg of body weight, in a range of about 0.01 mg/kg to about 10 mg/kg of body weight, in a range of about 0.01 mg/kg to about 9 mg/kg of body weight, in a range of about 0.01 mg/kg to about 8 mg/kg of body weight, in a range of about 0.01 mg/kg to about 7 mg/kg of body weight, in a range of 0.01 mg/kg to about 6 mg/kg of body weight, in a range of about 0.05 mg/kg to about 5 mg/kg of body weight, in a range of about 0.05 mg/kg to about 4 mg/kg of body weight, in a range of about 0.05 mg/kg to about 3 mg/kg of body weight, in a range of about 0.05 mg/kg to about 2 mg/kg of body weight, in a range of about 0.05 mg/kg to about 1.5 mg/kg of body weight, or in a range of about 0.05 mg/kg to about 1 mg/kg of body weight.

In accordance with certain aspects, the pharmaceutical composition (e.g., comprising a bacterial mixture) can be administered, for example, more than once daily, about once per day, about every other day, about every third day, about once a week, about once every two weeks, about once every month, about once every two months, about once every three months, about once every six months, or about once every year.

In an aspect, a pharmaceutical composition can be administered to a patient in need thereof at least once daily for at least two consecutive days. In another aspect, a pharmaceutical composition is administered at least once daily for at least 3, 4, 5, 6, 7, 8, 9, 10, 11, 12, 13, 14, or 15 consecutive days. In another aspect, a pharmaceutical composition is administered at least once daily for at least 1, 2, 3, 4, 5, 6, 7, 8, 9, 10, 11, or 12 consecutive weeks. In another aspect, a pharmaceutical composition is administered at least twice, three times, four times, or five times per week for at least 1, 2, 3, 4, 5, 6, 7, 8, 9, 10, 11, or 12 consecutive weeks. In another aspect, a pharmaceutical composition is administered at least once daily for at most 4, 5, 6, 7, 8, 9, 10, 11, 12, 13, 14, 15, 16, 17, 18, 19, or 20 consecutive days or weeks. In a further aspect, a pharmaceutical composition is administered at least once daily for at most 1, 2, 3, 4, 5, 6, 7, 8, 9, 10, 11, or 12 consecutive weeks or months. In yet another aspect, a pharmaceutical composition is administered at least once for at least 1, 2, 3, 4, 5, 6, 7, 8, 9, 10, 11, or 12 consecutive months or years, chronically for a subject's entire life span, or an indefinite period of time.

In an aspect, a pharmaceutical composition can be administered to a patient in need thereof at least twice daily for at least two consecutive days. In an aspect, a pharmaceutical composition is administered at least twice daily for at least 3, 4, 5, 6, 7, 8, 9, 10, 11, 12, 13, 14, or 15 consecutive days. In another aspect, a pharmaceutical composition is administered at least twice daily for at least 1, 2, 3, 4, 5, 6, 7, 8, 9, 10, 11, or 12 consecutive weeks. In another aspect, a pharmaceutical composition is administered at least twice daily for at most 4, 5, 6, 7, 8, 9, 10, 11, 12, 13, 14, 15, 16, 17, 18, 19, or 20 consecutive days or week. In another aspect, a pharmaceutical composition is administered at least twice daily for at most 1, 2, 3, 4, 5, 6, 7, 8, 9, 10, 11, or 12 consecutive weeks or months. In another aspect, a pharmaceutical composition is administered at least twice for at least 1, 2, 3, 4, 5, 6, 7, 8, 9, 10, 11, or 12 consecutive months or years, chronically for a subject's entire life span, or an indefinite period of time.

In an aspect of the present disclosure, a pharmaceutical composition can be administered to a patient in need thereof at least three times daily for at least two consecutive days. In an aspect, a pharmaceutical composition is administered at least three times daily for at least 3, 4, 5, 6, 7, 8, 9, 10, 11, 12, 13, 14, or 15 consecutive days. In an aspect, a pharmaceutical composition is administered at least three times daily for at least 1, 2, 3, 4, 5, 6, 7, 8, 9, 10, 11, or 12 consecutive weeks. In an aspect, a pharmaceutical composition is administered at least three times daily for at most 4, 5, 6, 7, 8, 9, 10, 11, 12, 13, 14, 15, 16, 17, 18, 19, or 20 consecutive days or weeks. In an aspect, a pharmaceutical composition is administered at least three times daily for at most 1, 2, 3, 4, 5, 6, 7, 8, 9, 10, 11, or 12 consecutive weeks or months. In an aspect, a pharmaceutical composition is administered at least three times for at least 1, 2, 3, 4, 5, 6, 7, 8, 9, 10, 11, or 12 consecutive months or years, chronically for a subject's entire life span, or an indefinite period of time.

In an aspect, a pharmaceutical composition can be administered to a patient in need thereof at a dosing schedule of at least once or twice daily for at least three consecutive days or weeks. In an aspect, a dose is administered at least once, twice, or three times daily for a period between 1 and 12 weeks, between 2 and 12 weeks, between 3 and 12 weeks, between 4 and 12 weeks, between 5 and 12 weeks, between 6 and 12 weeks, between 7 and 12 weeks, between 8 and 12 weeks, between 9 and 12 weeks, between 10 and 12 weeks, between 1 and 2 weeks, between 2 and 3 weeks, between 3 and 4 weeks, between 4 and 5 weeks, between 5 and 6 weeks, between 6 and 7 weeks, between 7 and 8 weeks, between 8 and 9 weeks, between 9 and 10 weeks, or between 10 and 11 weeks.

In an aspect, a pharmaceutical composition can be administered to a patient in need thereof at a dosing schedule of once-a-week, twice-a-week, or thrice-a-week. The term “once-a-week” means that a dose is administered typically only once in a week, for example, on the same day of each week. “Twice-a-week” means that a dose is administered typically only two times in a week, for example, on the same two days of each weekly period. “Thrice-a-week” means that a dose is administered typically only three times in a week, for example, on the same three days of each weekly period.

In an aspect, a pharmaceutical composition can be administered to a patient in need thereof, wherein the administration comprises a first dosing schedule followed by a second dosing schedule. In an aspect, a first dosing schedule comprises a treatment or induction dose. In an aspect, a second dosing schedule comprises a maintenance dose. For example, a pharmaceutically active maintenance dose of a second dosage schedule can be lower than or equal to a pharmaceutically active induction dose of a first dosing schedule. In other examples, a maintenance dose of a second dosing schedule can be higher than an induction dose of a first dosing schedule.

At least one of a first and second dosing schedule for administering a pharmaceutical composition can comprise administration of the composition at least once daily for at least one day. In an aspect, at least one of a first or second dosing schedule comprises administration of the composition at least once daily for at least 2, 3, 4, 5, 6, 7, 8, 9, 10, 11, 12, 13, 14, or 15 consecutive days. In an aspect, at least one of a first or second dosing schedule comprises administration of the composition at least once daily for at least 1, 2, 3, 4, 5, 6, 7, 8, 9, 10, 11, or 12 consecutive weeks. In an aspect, at least one of a first or second dosing schedule comprises administration of the composition for at most 4, 5, 6, 7, 8, 9, 10, 11, 12, 13, 14, 15, 16, 17, 18, 19, or 20 consecutive days or weeks. In an aspect, at least one of a first or second dosing schedule comprises administration of the composition for at most 1, 2, 3, 4, 5, 6, 7, 8, 9, 10, 11, or 12 consecutive weeks or months. In an aspect, at least one of a first or second dosing schedule comprises administration of the composition for at least 1, 2, 3, 4, 5, 6, 7, 8, 9, 10, 11, or 12 consecutive months or years, chronically for a subject's entire life span, or an indefinite period of time.

In an aspect, at least one of a first or second dosing schedule used in a method can be once-a-week, twice-a-week, or thrice-a-week.

In an aspect, at least one of a first and second dosing schedule can last for at least about 2, 4, 6, 8, 10, 12, 18, 24, 36, 48, 72, or 96 months. In an aspect, a second dosing schedule lasts permanently, for a treated subject's entire life span, or an indefinite period of time. In an aspect, at least one of a first and second dosing schedule is a continuous dosing schedule. In an aspect, at least one of a first and second dosing schedule is an intermittent dosing schedule. In an aspect, at least one of a first and second dosing schedule is an intermittent dosing schedule comprising a treatment period of at least 1, 2, 3, 4, 5, 6, 7, 8, 9, 10, 11, 12, 13, or 14 days followed by a resting period of at least 1, 2, 3, 4, 5, 6, 7, 8, 9, 10, 11, 12, 13, or 14 days. In an aspect, at least one of a first and second dosing schedule comprises administering a dose every other day, every two days, or every 3, 4, 5, 6, 7, 8 days. In an aspect, a dose is administered for an extended period of time with or without titration (or otherwise changing the dosage or dosing schedule).

In an aspect, the interval between a first and a second dosing schedule is at least about 1, 2, 3, 4, 5, 6, or 7 days, or at least about 1, 2, 3, 4, 5, 6, 7, 8, 9, 10, 11, or 12 weeks, or at least about 1, 2, 3, 4, 6, 7, 8, 9, 10, 11, or 12 months.

In an aspect, a second dosing schedule (e.g., a maintenance dose) comprises a dosage about 2, 3, 4, 5, 6, 7, 8, 9, 10, 20, 30, 40, 50, 75, 100, 200, 400, 800, 1000, 5000 or more fold lower than the dosage used in a first dosing schedule (e.g., an initial induction dose). In another aspect, a second dosing schedule (e.g., a maintenance dosing schedule) has an equal or lower dosing frequency than a first dosing schedule (e.g., an initial treatment dosing schedule). In an aspect, a second dosing schedule (e.g., a maintenance dosing schedule) has a higher dosing interval than a first dosing schedule (e.g., an initial treatment dosing schedule).

In various aspects, methods described herein are useful in treatment of a human subject. In some aspects, the human is a pediatric human. In other aspects, the human is an adult human. In other aspects, the human is a geriatric human. In other aspects, the human may be referred to as a patient. In some aspects, the human is a female. In some aspects, the human is a male.

In certain aspects, the human has an age in a range of from about 1 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

In one aspect, a subject being treated is a human patient. In one aspect, a patient is a male patient. In one aspect, a patient is a female patient. In one aspect, a patient is a premature newborn. In an aspect, a patient is a male premature newborn. In another aspect, a patient is a female premature newborn. In one aspect, a patient is a term newborn. In an aspect, a patient is a male term newborn. In another aspect, a patient is a female term newborn. In one aspect, a patient is a neonate. In one aspect, a patient is an infant. In another aspect, a patient is a male infant. In another aspect, a patient is a female infant. In one aspect, a patient is a toddler. In another aspect, a patient is a male toddler. In another aspect, a patient is a female toddler. In one aspect, a patient is a young child. In one aspect, a patient is a child. In another aspect, a patient is a male child. In another aspect, a patient is a female child. In one aspect, a patient is an adolescent. In one aspect, a patient is a pediatric patient. In another aspect, a patient is a male pediatric patient. In another aspect, a patient is a female pediatric patient. In one aspect, a patient is a geriatric patient. In another aspect, a patient is a male geriatric patient. In another aspect, a patient is a female geriatric patient. In one aspect, a patient is an adult male. In another aspect, the patient is an adult female. In one aspect, a human patient is a child patient below about 18, 15, 12, 10, 8, 6, 4, 3, 2, or 1 year old. In another aspect, a human patient is an adult patient. In another aspect, a human patient is an elderly patient. In a further aspect, a human patient is a patient above about 30, 35, 40, 45, 50, 55, 60, 65, 70, 75, 80, 85, 90, or 95 years old. In another aspect, a patient is about between 1 and 5, between 2 and 10, between 3 and 18, between 21 and 50, between 21 and 40, between 21 and 30, between 50 and 90, between 60 and 90, between 70 and 90, between 60 and 80, or between 65 and 75 years old. In one aspect, a patient is a young old patient (65-74 years). In one aspect, a patient is a middle old patient (75-84 years). In one aspect, a patient is an old patient (>85 years).

In one aspect, a subject being treated is a patient on a limited diet. In another aspect, a subject being treated is a patient on a non-limited diet. In another aspect, a subject being treated is a patient on a diet comprising animal protein. In another aspect, a subject being treated is a patient on a diet comprising spicy foods. In another aspect, a subject being treated is a patient on a diet comprising high fat food.

In one aspect, a method comprises administering a therapeutic composition orally, by enema, or via rectal suppository. In one aspect, a therapeutic composition administered herein is formulated as an enteric coated (and/or acid-resistant) capsule or microcapsule, or formulated as part of or administered together with a food, a food additive, a dairy-based product, a soy-based product or a derivative thereof, a jelly, flavored liquid, ice block, ice cream, or a yogurt. In another aspect, a therapeutic composition administered herein is formulated as an acid-resistant enteric coated capsule. A therapeutic composition can be provided as a powder for sale in combination with a food or drink. A food or drink can be a dairy-based product or a soy-based product. In another aspect, a food or food supplement contains enteric-coated and/or acid-resistant microcapsules containing a therapeutic composition.

In an aspect, a therapeutic composition comprises a liquid culture. In another aspect, a therapeutic composition is homogenized, lyophilized, pulverized and powdered. It may then be infused, dissolved such as in saline, as an enema. Alternatively the powder may be encapsulated as enteric-coated and/or acid-resistant delayed release capsules for oral administration. In an aspect, the powder may be double encapsulated with acid-resistant/delayed release capsules for oral administration. These capsules may take the form of enteric-coated and/or acid-resistant delayed release microcapsules. A powder can preferably be provided in a palatable form for reconstitution for drinking or for reconstitution as a food additive. In a further aspect, a food is yogurt. In one aspect, a powder may be reconstituted to be infused via naso-duodenal infusion.

In another aspect, a therapeutic composition administered herein is in a liquid, frozen, freeze-dried, spray-dried, foam-dried, lyophilized, or powder form. In a further aspect, a therapeutic composition administered herein is formulated as a delayed or gradual enteric release form. In another aspect, a therapeutic composition administered herein comprises an excipient, a saline, a buffer, a buffering agent, or a fluid-glucose-cellobiose agar (RGCA) media. In another aspect, a therapeutic composition administered herein comprises a cryoprotectant. In one aspect, a cryoprotectant comprises polyethylene glycol, skim milk, erythritol, arabitol, sorbitol, glucose, fructose, alanine, glycine, proline, sucrose, lactose, ribose, trehalose, dimethyl sulfoxide (DMSO), glycerol, or a combination thereof.

In one aspect, a pharmaceutical composition comprises a lyophilized formulation further comprising a reducing agent. In certain embodiments, the reducing agent comprises cysteine selected from the group consisting of D-cysteine and L-cysteine. In another aspect, cysteine is at a concentration of at least about 0.025%. In one aspect, cysteine is at a concentration of about 0.025%. In another aspect, cysteine is at a concentration of 0.025%. In another aspect, another reducing agent other than cysteine is used in lieu of, or in combination with cysteine. In an aspect, another reducing agent is selected from the group comprising ascorbic acid, sodium ascorbate, thioglycolic acid, sodium sulfite, sodium bisulfite, sodium metabisulfite, potassium metabisulfite, Glutathione, Methionine, thioglycerol, and alpha tocopherol.

In one aspect, cysteine is at a concentration of at least about 0.005%, at least about 0.01%, at least about 0.015%, at least about 0.02%, at least about 0.025%, at least about 0.03%, at least about 0.035%, at least about 0.04%, at least about 0.045%, at least about 0.05%, at least about 0.055%, at least about 0.06%, at least about 0.065%, at least about 0.07%, at least about 0.075%, at least about 0.08%, at least about 0.085%, at least about 0.09%, at least about 0.095%, at least about 0.1%, at least about 0.12%, at least about 0.14%, at least about 0.16%, at least about 0.18%, at least about 0.2%, at least about 0.25%, at least about 0.3%, at least about 0.4%, at least about 0.5%, at least about 0.6%, at least about 0.7%, at least about 0.8%, at least about 0.9%, at least about 1%, at least about 2%, at least about 4%, at least about 6%, at least about 8%, at least about 10%, at least about 12%, at least about 14%, at least about 16%, at least about 18%, at least about 20%, at least about 22%, at least about 24%, or at least about 26%.

In one aspect, a therapeutic composition comprises a cryoprotectant. As used herein, a “cryoprotectant” refers to a substance that is added to a formulation in order to protect an active ingredient during freezing. In an aspect, a cryoprotectant comprises, consists essentially of, or consists of polyethylene glycol, skim milk, erythritol, arabitol, sorbitol, glucose, fructose, alanine, glycine, proline, sucrose, lactose, ribose, trehalose, dimethyl sulfoxide (DMSO), glycerol, or a combination thereof. In an aspect of the present disclosure, a cryoprotectant can be selected from the group comprising 5% Sucrose; 10% Sucrose; 10% Skim milk; 10% Trehalose with 2.5% sucrose; 5% Trehalose with 2.5% sucrose; 5% Mannitol; 5% Mannitol with 0.1% Polysorbate 80; 10% Mannitol; 10% Mannitol with 0.1% Polysorbate 80; 5% Trehalose; 5% Trehalose with 0.1% Polysorbate 80; 10% Trehalose; and 10% Trehaolse with 0.1% Polysorbate 80.

In another aspect, a therapeutic composition comprises a lyoprotectant. As used herein, a “lyoprotectant” refers to a substance that is added to a formulation in order to protect an active ingredient during the drying stage of a lyophilization (also known as freeze-drying) process. In one aspect, the same substance or the same substance combination is used as both a cryoprotectant and a lyoprotectant. Exemplary lyoprotectants include sugars such as sucrose or trehalose; an amino acid such as monosodium glutamate or histidine; a methylamine such as betaine; a lyotropic salt such as magnesium sulfate; a polyol such as trihydric or higher sugar alcohols, e.g. glycerin, erythritol, glycerol, arabitol, xylitol, sorbitol, and mannitol; propylene glycol; polyethylene glycol; Pluronics; and combinations thereof. In one aspect, a lyoprotectant is a non-reducing sugar, such as trehalose or sucrose. In one aspect, a cryoprotectant or a lyoprotectant consists essentially of, or consists of, one or more substances mentioned in this paragraph and the paragraph above.

In one aspect, a cryoprotectant or a lyoprotectant comprise an intracellular agent, e.g., DMSO, Glycerol, or PEG, which penetrates inside the cell preventing the formation of ice crystals that could result in membrane rupture. In another aspect, a cryoprotectant or a lyoprotectant comprise an extracellular agent, e.g., sucrose, trehalose, or dextrose, which does not penetrate into the cell membrane but acts to improve the osmotic imbalance that occurs during freezing.

In one aspect, the present disclosure provides a pharmaceutical composition comprising a lyophilized fecal microbe preparation comprising a lyophilization formulation comprising at least about 12.5% trehalose.

In an aspect, a lyophilized formulation comprises trehalose. In an aspect, a lyophilized formulation comprises 2% to 30%, 3% to 25%, 4% to 20%, 5% to 15%, 6% to 10%, 2% to 30%, 2% to 25%, 2% to 20%, 2% to 15%, or 2% to 10% trehalose. In an aspect, a lyophilized formulation comprises at least 2%, 3%, 4%, 5%, 6%, 7%, 8%, 9%, 10%, or 15% trehalose. In an aspect, a lyophilized formulation comprises at most 2%, 3%, 4%, 5%, 6%, 7%, 8%, 9%, 10%, or 15% trehalose. In another aspect, a lyophilized formulation comprises about 5% trehalose. In another aspect, a lyophilized formulation comprises trehalose and sucrose. In another aspect, a lyophilized formulation comprises between about 8% and 12% trehalose with between about 1.5% and 3.5% sucrose and between about 0.5% and 1.5% NaCl.

In one aspect, a lyophilization formulation comprises at least about 5%, at least about 7.5%, at least about 10%, at least about 12.5%, at least about 13%, at least about 13.5%, at least about 14%, at least about 14.5%, at least about 15%, at least about 15.5%, at least about 16%, at least about 16.5%, at least about 17%, at least about 17.5%, at least about 18%, at least about 18.5%, at least about 19%, at least about 19.5%, at least about 20%, at least about 22.5%, at least about 25%, at least about 27.5%, at least about 30%, at least about 32.5%, at least about 35%, at least about 37.5%, at least about 40%, at least about 42.5%, at least about 45%, at least about 47.5%, at least about 50%, at least about 52.5%, at least about 55%, at least about 57.5%, or at least about 60% of trehalose.

In one aspect, a therapeutic composition administered herein further comprises an acid suppressant, an antacid, an H2 antagonist, a proton pump inhibitor or a combination thereof. In one aspect, a therapeutic composition administered herein substantially free of non-living matter. In another aspect, a therapeutic composition administered herein substantially free of acellular material selected from the group consisting of residual fiber, DNA, viral coat material, and non-viable material. In another aspect, a therapeutic composition administered does not comprise an acid suppressant, an antacid, an H2 antagonist, a proton pump inhibitor or a combination thereof. In yet another aspect, a therapeutic composition administered does not comprise an acid suppressant. In another aspect, a therapeutic composition administered does not comprise an antacid. In another aspect, a therapeutic composition administered does not comprise an H2 antagonist. In another aspect, a therapeutic composition administered does not comprise a proton pump inhibitor. In another aspect, a therapeutic composition administered does not comprise metoclopramide.

In one aspect, a therapeutic composition also comprises or is supplemented with a prebiotic nutrient selected from the group consisting of polyols, fructooligosaccharides (FOSs), oligofructoses, inulins, galactooligosaccharides (GOSs), xylooligosaccharides (XOSs), polydextroses, monosaccharides, tagatose, and/or mannooligosaccharides. In another aspect, a subject is not pretreated with a prebiotic nutrient prior to treatment with a therapeutic composition. In another aspect, the therapeutic composition is not supplemented with a prebiotic nutrient.

In one aspect, a method further comprises pretreating a subject with an antibiotic composition prior to administering a therapeutic bacterial or microbiota composition. In one aspect, an antibiotic composition administered herein comprises an antibiotic selected from the group consisting of rifabutin, clarithromycin, clofazimine, vancomycin, rifampicin, nitroimidazole, chloramphenicol, and a combination thereof. In another aspect, an antibiotic composition administered herein comprises an antibiotic selected from the group consisting of rifaximin, rifamycin derivative, rifampicin, rifabutin, rifapentine, rifalazil, bicozamycin, aminoglycoside, gentamycin, neomycin, streptomycin, paromomycin, verdamicin, mutamicin, sisomicin, netilmicin, retymicin, kanamycin, aztreonam, aztreonam macrolide, clarithromycin, dirithromycin, roxithromycin, telithromycin, azithromycin, bismuth subsalicylate, vancomycin, streptomycin, fidaxomicin, amikacin, arbekacin, neomycin, netilmicin, paromomycin, rhodostreptomycin, tobramycin, apramycin, and a combination thereof. In another aspect, a subject is not pretreated with an antibiotic composition prior to administering a therapeutic bacterial or microbiota composition. In another aspect, the therapeutic composition is not supplemented with an antibiotic composition. In a further aspect, a method further comprises pretreating a subject with an anti-inflammatory drug prior to administration of a therapeutic bacterial or microbiota composition. In yet another aspect, a subject is not pretreated with an anti-inflammatory drug prior to administering a therapeutic bacterial or microbiota composition. In another aspect, a therapeutic bacterial or microbiota composition is not supplemented with an anti-inflammatory.

In an aspect of the present disclosure, a method further comprises administering a therapeutic bacterial or microbiota composition to a subject in need thereof, without co-administering steroids. In another aspect, the subject has not been previously treated with steroids to treat a dysbiosis. In yet another aspect, the subject is not administered a steroid at least 1, 2, 3, 4, 5, 6, 10, 15, 20, 25, 30, 35, 40, 45, or 50 weeks prior to the administering of a therapeutic composition. In a further aspect, the subject is not administered a steroid at least 1, 2, 3, 4, 5, 6, 7, 7, 9, or 10 years prior to the administering of a therapeutic composition. In yet another aspect, the subject is not treated with steroids for at least 1, 2, 3, or 4 weeks prior to or after the administering of a therapeutic composition comprising fecal microbiota. In another aspect, the subject is not co-treated with drugs to treat conditions of dysbiosis (e.g., Crohn's disease, Ulcerative Colitis, Irritable Bowel Disease, etc.). In yet another aspect, a subject is not co-treated with thiopurines or 5-aminoscalicylate (5-ASA). In a further aspect, a subject is not co-treated with a corticosteroid, 5-ASA products, immunomodulators, anti-TNFα agents, or other medication prescribed to treat Crohn's disease, Ulcerative Colitis, Irritable Bowel Syndrome, and Irritable Bowel Disease. In another aspect, a subject is not co-treated with a drug used to treat gastrointestinal disorders.

In an aspect of the present disclosure, a method further comprises administering a therapeutic bacterial or microbiota composition to a subject in need thereof, without co-administering nonsteroidal anti-inflammatory drugs. In another aspect, the subject has not been previously treated with nonsteroidal anti-inflammatory drugs to prevent ulcerative colitis flare-ups. In yet another aspect, the subject is not administered a nonsteroidal anti-inflammatory drug at least 1, 2, 3, 4, 5, 6, 10, 15, 20, 25, 30, 35, 40, 45, or 50 weeks prior to the administering of a therapeutic composition. In a further aspect, the subject is not administered a nonsteroidal anti-inflammatory drug at least 1, 2, 3, 4, 5, 6, 7, 7, 9, or 10 years prior to the administering of a therapeutic composition. In yet another aspect, the subject is not treated with nonsteroidal anti-inflammatory drug for at least 1, 2, 3, or 4 weeks prior to or after the administering of a therapeutic composition comprising fecal microbiota. In another aspect, the subject is not treated with mesalamine for at least 1, 2, 3, or 4 weeks prior to or after the administering of a therapeutic composition comprising fecal microbiota.

The compositions and methods of the present invention may further comprise one or more prebiotics.

A prebiotic is a substrate that is selectively used by a host microorganism to produce a health benefit in a subject. Without wishing to be bound by theory, prebiotics are added to nutritionally supplement bacteria in the microbiome and/or in a microbial composition, e.g., to stimulate the growth or activity of one or more strains of beneficial bacteria. Additionally, the prebiotics may be added to prevent “shock” to bacterial strains subsequent to their isolation or purification, freezing, freeze-drying, spray-drying, reconstitution in solution and the like.

Examples of prebiotics include amino acids, ammonium nitrate, amylose, barley mulch, biotin, carbonate, cellulose, chitin, choline, fructooligosaccharides (FOSs), fructose, galactooligosaccharides (GOSs), glucose, glycerol, heteropolysaccharide, histidine, homopolysaccharide, hydroxyapatite, inulin, isomaltulose, lactose, lactulose, maltodextrins, maltose, mannooligosaccharides, tagatose, nitrogen, oligodextrose, oligofructoses, oligofructose-enriched inulin, oligosaccharides, pectin, phosphate salts, phosphorus, polydextroses, polyols, potash, potassium, sodium nitrate, starch, sucrose, sulfur, sun fiber, tagatose, thiamine, trans-galactooligosaccharides, trehalose, vitamins, a water-soluble carbohydrate, and/or xylooligosaccharides (XOSs).

In embodiments, a prebiotic can be added (e.g., in dry or liquid forms) to a microbial composition of the present invention.

Alternately, or additionally, a prebiotic can be included (e.g., in dry or liquid forms) in a distinct pharmaceutical composition which lacks a microbial composition of the present invention.

A prebiotic may be provided to a subject before, contemporaneously with, and/or after a pharmaceutical composition comprising a microbial composition of the present invention is administered, either in a pharmaceutical composition comprising the microbial composition or in a pharmaceutical composition lacking a microbial composition.

A prebiotic may be provided in a single dose or in multiple doses. When provided as a single composition, the single composition may comprise a single prebiotic or a mixture of prebiotics. When provided in multiple compositions, each composition may comprise a single prebiotic or a mixture of prebiotics.

As examples, when multiple doses are provided, a first composition comprising a prebiotic may include one specific prebiotic, e.g., inulin, and a second composition may include a second specific prebiotic, e.g., pectin. Alternately, a first composition may include a mixture of prebiotics, e.g., inulin and pectin and a second composition may include different mixture of prebiotics, e.g., inulin and a FOS. A first composition may include a mixture of prebiotics and a second composition may include one specific prebiotic.

The amount of prebiotic provided to a subject/patient and/or included in a composition depends on the specific prebiotic, the specific bacterial strain of beneficial bacteria, and/or the disease state of the subject.

In one aspect, every about 200 mg of a pharmaceutical composition comprises a pharmacologically active dose. In one aspect, every about 75, 100, 125, 150, 175, 200, 250, 300, 350, 400, 450, 500, 750, 1000, 1500, or 2000 mg of a pharmaceutical composition comprises a pharmacologically active dose.

In one aspect, a pharmaceutically active or therapeutic effective dose comprises at least about 10⁵, 10⁶, 10⁷, 10⁸, 10⁹, 10¹⁰, 10¹¹, 10¹², 10¹³, 10¹⁴, or 10¹⁵ cfu. In another aspect, a pharmaceutically active therapeutic effective dose comprises at most about 10⁵, 10⁶, 10⁷, 10⁸, 10⁹, 10¹⁰, 10¹¹, 10¹², 10¹³, 10¹⁴, or 10¹⁵ cfu. In a further aspect, a pharmacologically active therapeutic effective dose is selected from the group consisting of from 10⁸ cfu to 10¹⁴ cfu, from 10⁹ cfu to 10¹³ cfu, from 10¹⁰ cfu to 10¹² cfu, from 10⁹ cfu to 10¹⁴ cfu, from 10⁹ cfu to 10¹² cfu, from 10⁹ cfu to 10¹¹ cfu, from 10⁹ cfu to 10¹⁰ cfu, from 10¹⁰ cfu to 10¹⁴ cfu, from 10¹⁰ cfu to 10¹³ cfu, from 10¹¹ cfu to 10¹⁴ cfu, from 10¹¹ cfu to 10¹³ cfu, from 10¹² cfu to 10¹⁴ cfu, and from 10¹³ cfu to 10¹⁴ cfu. In one aspect, a pharmaceutical composition comprises the foregoing pharmaceutically active or therapeutic effective dose in a unit weight of about 0.2, 0.4, 0.6, 0.8 or 1.0 gram, or a unit volume of about 0.2, 0.4, 0.6, 0.8 or 1.0 milliliter.

In one aspect, a pharmaceutically active or therapeutic effective dose comprises at least about 10⁵, 10⁶, 10⁷, 10⁸, 10⁹, 10¹⁰, 10¹¹, 10¹², 10¹³, 10¹⁴, or 10¹⁵ cells or spores. In another aspect, a pharmaceutically active or therapeutic effective dose comprises at most about 10⁵, 10⁶, 10⁷, 10⁸, 10⁹, 10¹⁰, 10¹¹, 10¹², 10¹³, 10¹⁴, or 10¹⁵ total cells or spores. In a further aspect, a pharmacologically active or therapeutic effective dose is selected from the group consisting of from 10⁸ to 10¹⁴, from 10⁹ to 10¹³, from 10¹⁰ to 10¹², from 10⁹ to 10¹⁴, from 10⁹ to 10¹², from 10⁹ to 10¹¹, from 10⁹ to 10¹⁰, from 10¹⁰ to 10¹⁴, from 10¹⁰ to 10¹³, from 10¹¹ to 10¹⁴, from 10¹¹ to 10¹³, from 10¹² to 10¹⁴, and from 10¹³ to 10¹⁴ cells or spores. In an aspect, the pharmaceutically active or therapeutic effective dose cell count is directed to live cells. In one aspect, a pharmaceutical composition comprises the foregoing pharmaceutically active or therapeutic effective dose in a unit weight of about 0.2, 0.4, 0.6, 0.8 or 1.0 gram, or a unit volume of about 0.2, 0.4, 0.6, 0.8 or 1.0 milliliter. In an aspect, a pharmaceutically active or therapeutic effective dose comprises between 10¹⁰ and 10¹² cells. In another aspect, a pharmaceutically active or therapeutic effective dose comprises between 10¹⁰ and 10¹² cells per capsule. In another aspect, a pharmaceutically active or therapeutic effective dose comprises between 10¹¹ and 10¹² cells per capsule. In a further aspect, a pharmaceutically active or therapeutic effective dose comprises between 10⁹ and 10¹² cells per capsule.

In one aspect, a therapeutic composition administered herein comprises fecal bacteria. In one aspect, a therapeutic composition administered herein comprises one or more, two or more, three or more, four or more, or five or more isolated, purified, or cultured microorganisms selected from the group consisting of Clostridium, Bacillus, Collinsella, Bacteroides, Eubacterium, Fusobacterium, Propionibacterium, Lactobacillus, Ruminococcus, Escherichia coli, Gemmiger, Desulfomonas, Peptostreptococcus, Bifidobacterium, Coprococcus, Dorea, and Monilia.

In one aspect, a therapeutic composition administered herein comprises at least one, at least two, at least three, at least four, at least five, at least six, or at least seven fecal microorganisms selected from the group consisting of a Bacteroides fragilis ssp. vulgatus, Collinsella aerofaciens, Bacteroides fragilis ssp. thetaiotaomicron, Peptostreptococcus productus II, Parabacteroides distasonis, Fecalibacterium prausnitzii, Coprococcus eutactus, Collinsella aerofaciens III, Peptostreptococcus productus I, Ruminococcus bromii, Bifidobacterium adolescentis, Gemmiger formicilis, Bifidobacterium longum, Eubacterium siraeum, Ruminococcus torques, Eubacterium rectale, Eubacterium eligens, Bacteroides eggerthii, Clostridium leptum, Bacteroides fagilis ssp. A, Eubacterium biforme, Bifidobacterium infantis, Eubacterium rectale III-F, Coprococcus comes, Pseudoflavonifractor capillosus, Ruminococcus albus, Dorea formicigenerans, Eubacterium hallii, Eubacterium ventriosum I, Fusobacterium russi, Ruminococcus obeum, Eubacterium rectale, Clostridium ramosum, Lactobacillus leichmannii, Ruminococcus callidus, Butyrivibrio crossotus, Acidaminococcus fermentans, Eubacterium ventriosum, Bacteroides fragilis ssp. fragilis, Bacteroides AR, Coprococcus catus, Aerostipes hadrus, Eubacterium cylindroides, Eubacterium ruminantium, Eubacterium CH-1, Staphylococcus epidermidis, Peptostreptococcus BL, Eubacterium limosum, Tissirella praeacuta, Bacteroides L, Fusobacterium mortiferum I, Fusobacterium naviforme, Clostridium innocuum, Clostridium ramosum, Propionibacterium acnes, Ruminococcus flavefaciens, Ruminococcus AT, Peptococcus AU-1, Bacteroides fagilis ssp. ovatus, -ssp. d, -ssp. f, Bacteroides L-1, L-5; Fusobacterium nucleatum, Fusobacterium mortiferum, Escherichia coli, Gemella morbillorum, Finegoldia magnus, Peptococcus G, -AU-2; Streptococcus intermedius, Ruminococcus lactaris, Ruminococcus CO Gemmiger X, Coprococcus BH, —CC; Eubacterium tenue, Eubacterium ramulus, Bacteroides clostridiiformis ssp. clostridliformis, Bacteroides coagulans, Prevotella oralis, Prevotella ruminicola, Odoribacter splanchnicus, Desuifomonas pigra, Lactobacillus G, Succinivibrio A, and a combination thereof.

In one aspect, a therapeutic composition administered herein comprises no viable Bacteroides, Fusobacterium, Propionibacterium, Lactobacillus, Ruminococcus, Escherichia coli, Gemmiger, Desulfomonas, Peptostreptococcus, Bifidobacterium, Monilia, or any combination thereof. In another aspect, a therapeutic composition administered herein comprises no viable Bacteroides fragilis ssp. vulgatus, Collinsella aerofaciens, Bacteroides fragilis ssp. thetaiotaomicron, Peptostreptococcus productus II, Parabacteroides distasonis, Fecalibacterium prausnitzii, Coprococcus eutactus, Collinsella aerofaciens III, Peptostreptococcus productus I, Ruminococcus bromii, Bifidobacterium adolescentis, Gemmiger formicilis, Bifidobacterium longum, Eubacterium siraeum, Ruminococcus torques, Eubacterium rectale, Eubacterium eligens, Bacteroides eggerthii, Clostridium leptum, Bacteroides fragilis ssp. A, Eubacterium biforme, Bifidobacterium infantis, Eubacterium rectale III-F, Coprococcus comes, Pseudoflavonifractor capillosus, Ruminococcus albus, Dorea formicigenerans, Eubacterium hallii, Eubacterium ventriosum I, Fusobacterium russi, Ruminococcus obeum, Eubacterium rectale, Clostridium ramosum, Lactobacillus leichmannii, Ruminococcus callidus, Butyrivibrio crossotus, Acidaminococcus fermentans, Eubacterium ventriosum, Bacteroides fragilis ssp. fragilis, Bacteroides AR, Coprococcus catus, Aerostipes hadrus, Eubacterium cylindroides, Eubacterium ruminantium, Eubacterium CH-1, Staphylococcus epidermidis, Peptostreptococcus BL, Eubacterium limosum, Tissirella praeacuta, Bacteroides L, Fusobacterium mortiferum I, Fusobacterium naviforme, Clostridium innocuum, Clostridium ramosum, Propionibacterium acnes, Ruminococcus flavefaciens, Ruminococcus AT, Peptococcus AU-1, Bacteroides fagilis ssp. ovatus, -ssp. d, -ssp. f, Bacteroides L-1, L-5; Fusobacterium nucleatum, Fusobacterium mortiferum, Escherichia coli, Gemella morbillorum, Finegoldia magnus, Peptococcus G, -AU-2; Streptococcus intermedius, Ruminococcus lactaris, Ruminococcus CO Gemmiger X, Coprococcus BH, -CC; Eubacterium tenue, Eubacterium ramulus, Bacteroides clostridiiformis ssp. clostridliformis, Bacteroides coagulans, Prevotella oralis, Prevotella ruminicola, Odoribacter splanchnicus, Desuifomonas pigra, Lactobacillus G, Succinivibrio A, or a combination thereof.

In one aspect, a therapeutic composition administered herein comprises a fecal microbiota. In another aspect, the preparation of a fecal microbiota used herein involves a treatment selected from the group consisting of ethanol treatment, detergent treatment, heat treatment, irradiation, and sonication. In another aspect, the preparation of a fecal microbiota used herein involves no treatment selected from the group consisting of ethanol treatment, detergent treatment, heat treatment, irradiation, and sonication. In one aspect, the preparation of a fecal microbiota used herein involves a separation step selected from the group consisting of density gradients, filtration (e.g., sieves, nylon mesh), and chromatography. In another aspect, the preparation of a fecal microbiota used herein involves no separation step selected from the group consisting of density gradients, filtration (e.g., sieves, nylon mesh), and chromatography. In another aspect, a fecal microbiota used herein comprises a donor's entire fecal microbiota. In another aspect, a therapeutic composition administered herein comprises a fecal microbiota substantially free of eukaryotic cells from the fecal microbiota's donor.

In an aspect, the preparation of uncultured fecal bacteria from stool of a donor involves a treatment selected from the group consisting of ethanol treatment, detergent treatment, heat treatment, irradiation, and sonication. In another aspect, the preparation of uncultured fecal bacteria from stool of a donor involves no treatment selected from the group consisting of ethanol treatment, detergent treatment, heat treatment, irradiation, and sonication. In one aspect, the preparation of uncultured fecal bacteria from stool of a donor involves a separation step selected from the group consisting of density gradients, filtration (e.g., sieves, nylon mesh), and chromatography. In another aspect, the preparation of uncultured fecal bacteria from stool of a donor involves no separation step selected from the group consisting of density gradients, filtration (e.g., sieves, nylon mesh), and chromatography. In another aspect, a preparation of uncultured fecal bacteria comprises an entire or substantially entire fecal microbiota from a stool sample of a donor. In another aspect, a pharmaceutical composition administered herein comprises a preparation of uncultured fecal bacteria substantially free of donor eukaryotic cells.

In another aspect, a therapeutic composition administered herein comprises a fecal microbiota further supplemented, spiked, or enhanced with a fecal microorganism. In one aspect, a fecal microbiota is supplemented with a non-pathogenic (or with attenuated pathogenicity) bacterium of Clostridium, Collinsella, Dorea, Ruminococcus, Coprococcus, Prevotella, Veillonella, Bacteroides, Baccillus, or a combination thereof. In another aspect, a therapeutic composition administered herein comprises a fecal microbiota further supplemented, spiked, or enhanced with a species of Veillonellaceae, Firmicutes, Gammaproteobacteria, Bacteroidetes, or a combination thereof. In another aspect, a therapeutic composition administered herein comprises a fecal microbiota further supplemented with fecal bacterial spores. In one aspect, fecal bacterial spores are Clostridium spores, Bacillus spores, or both.

In an aspect, a therapeutic composition comprises a fecal microbiota from a subject selected from the group consisting of a human, a bovine, a dairy calf, a ruminant, an ovine, a caprine, or a cervine. In another aspect, a therapeutic composition can be administered to a subject selected from the group consisting of a human, a bovine, a dairy calf, a ruminant, an ovine, a caprine, or a cervine. In an aspect, a therapeutic composition is substantially or nearly odourless.

In an aspect, a therapeutic composition provided or administered herein comprises a fecal microbiota comprising a Shannon Diversity Index of greater than or equal to 0.3, greater than or equal to 0.4, greater than or equal to 0.5, greater than or equal to 0.6, greater than or equal to 0.7, greater than or equal to 0.8, greater than or equal to 0.9, greater than or equal to 1.0, greater than or equal to 1.1, greater than or equal to 1.2, greater than or equal to 1.3, greater than or equal to 1.4, greater than or equal to 1.5, greater than or equal to 1.6, greater than or equal to 1.7, greater than or equal to 1.8, greater than or equal to 1.9, greater than or equal to 2.0, greater than or equal to 2.1, greater than or equal to 2.2, greater than or equal to 2.3, greater than or equal to 2.4, greater than or equal to 2.5, greater than or equal to 3.0, greater than or equal to 3.1, greater than or equal to 3.2, greater than or equal to 3.3, greater than or equal to 3.4, greater than or equal to 3.5, greater than or equal to 3.6, greater than or equal to 3.7, greater than or equal to 3.8, greater than or equal to 3.9, greater than or equal to 4.0, greater than or equal to 4.1, greater than or equal to 4.2, greater than or equal to 4.3, greater than or equal to 4.4, greater than or equal to 4.5, or greater than or equal to 5.0. In another aspect, a therapeutic composition comprises fecal microbiota comprising a Shannon Diversity Index of between 0.1 and 3.0, between 0.1 and 2.5, between 0.1 and 2.4, between 0.1 and 2.3, between 0.1 and 2.2, between 0.1 and 2.1, between 0.1 and 2.0, between 0.4 and 2.5, between 0.4 and 3.0, between 0.5 and 5.0, between 0.7 and 5.0, between 0.9 and 5.0, between 1.1 and 5.0, between 1.3 and 5.0, between 1.5 and 5.0, between 1.7 and 5.0, between 1.9 and 5.0, between 2.1 and 5.0, between 2.3 and 5.0, between 2.5 and 5.0, between 2.7 and 5.0, between 2.9 and 5.0, between 3.1 and 5.0, between 3.3 and 5.0, between 3.5 and 5.0, between 3.7 and 5.0, between 31.9 and 5.0, or between 4.1 and 5.0. In one aspect, a Shannon Diversity Index is calculated at the phylum level. In another aspect, a Shannon Diversity Index is calculated at the family level. In one aspect, a Shannon Diversity Index is calculated at the genus level. In another aspect, a Shannon Diversity Index is calculated at the species level. In a further aspect, a therapeutic composition comprises a preparation of flora in proportional content that resembles a normal healthy human fecal flora.

In a further aspect, a therapeutic composition comprises fecal bacteria from at least 1, 2, 3, 4, 5, 6, 7, 8, 9, or 10 different families. In another aspect, a therapeutic composition comprises fecal bacteria from at least 11, 12, 13, 14, 15, 16, 17, 18, 19, or 20 different families. In yet another aspect, a therapeutic composition comprises fecal bacteria from at least 21, 22, 23, 24, 25, 26, 27, 28, 29, or 30 different families. In a further aspect, a therapeutic composition comprises fecal bacteria from at least 31, 32, 33, 34, 35, 36, 37, 38, 39, or 40 different families. In another aspect, a therapeutic composition comprises fecal bacteria from at least 41, 42, 43, 44, 45, 46, 47, 48, 49, or 50 different families. In another aspect, a therapeutic composition comprises fecal bacteria from between 1 and 10, between 10 and 20, between 20 and 30, between 30 and 40, between 40 and 50 different families. In an aspect, a therapeutic composition provided or administered herein comprises a fecal microbiota comprising no greater than 0.05%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, or 10% weight non-living material/weight biological material. In another aspect, a therapeutic composition provided or administered herein comprises a fecal microbiota comprising no greater than 20%, 25%, 30%, 35%, 40%, 45%, 50%, 55%, 60%, 65%, 70%, 75%, 80%, 85%, 90%, or 95% weight non-living material/weight biological material.

In another aspect, a pharmaceutical composition provided or administered herein comprises, consists of, or consists essentially of, particles of non-living stool material and/or particles of biological material of a fecal sample that passes through a sieve, a column, or a similar filtering device having a sieve, exclusion, or particle filter size of 2.0 mm, 1.0 mm, 0.5 mm, 0.33 mm, 0.25 mm, 0.212 mm, 0.180 mm, 0.150 mm, 0.125 mm, 0.106 mm, 0.090 mm, 0.075 mm, 0.063 mm, 0.053 mm, 0.045 mm, 0.038 mm, 0.032 mm, 0.025 mm, 0.020 mm, 0.01 mm, or 0.002 mm. “Non-living stool material” refers to material present in stool when the stool is collected from a donor, and does not include an excipient, e.g., a pharmaceutically inactive substance, such as a cryoprotectant, added during processing of fecal material. “Biological material” refers to the living material in fecal material, and includes microbes including prokaryotic cells, such as bacteria and archaea (e.g., living prokaryotic cells and spores that can sporulate to become living prokaryotic cells), eukaryotic cells such as protozoa and fungi, and viruses. In one aspect, “biological material” refers to the living material, e.g., the microbes, eukaryotic cells, and viruses, which are present in the intestine (e.g., colon) of a normal healthy human. In an aspect, a pharmaceutical composition provided or administered herein comprises an extract of human stool, wherein the composition is substantially odorless. In an aspect, a pharmaceutical composition provided or administered herein comprises fecal material or a fecal floral preparation in a lyophilized, crude, semi-purified or purified formulation.

In an aspect, a preparation of uncultured fecal bacteria included in a pharmaceutical composition comprises highly refined or purified fecal flora, e.g., substantially free of non-floral fecal material. In an aspect, a fecal microbiota (comprising uncultured fecal bacteria) harvested from a donor can be further processed, e.g., to undergo microfiltration before, after, or before and after sieving. In another aspect, a highly purified fecal microbiota product is ultra-filtrated to remove large molecules but retain the therapeutic microflora, e.g., bacteria.

In another aspect, a preparation of uncultured fecal bacteria incorporated into a pharmaceutical composition described herein comprises or consists essentially of a substantially isolated or a purified fecal flora or entire (or substantially entire) microbiota that is (or comprises) an isolate of fecal flora that is at least about 90%, 91%, 92%, 93%, 94%, 95%, 96%, 97%, 98%, 99%, 99.5%, 99.6%, 99.7%, 99.8% or 99.9% isolated or pure, or having no more than about 0.1%, 0.2%, 0.3%, 0.4%, 0.5%, 0.6%, 0.7%, 0.8%, 0.9% or 1.0% or more non-fecal floral material; or, a substantially isolated, purified, or substantially entire microbiota as described in Sadowsky et al., WO 2012/122478 A1, or as described in Borody et al., WO 2012/016287 A2.

In an aspect, a preparation of uncultured fecal bacteria included in a pharmaceutical composition comprises the substantially entire fecal microbiota of stool of a donor. In another aspect, uncultured fecal bacteria of a pharmaceutical composition comprise no antibiotic resistant bacteria. In another aspect, a pharmaceutical composition comprises a preparation of uncultured fecal bacteria largely free of extraneous matter (e.g., non-living matter including acellular matter such as residual fiber, DNA, RNA, viral coat material, non-viable material; and living matter such as eukaryotic cells from the donor of the fecal matter).

In an aspect, uncultured fecal bacteria included in a pharmaceutical composition are derived from a disease-screened stool sample of a human donor. In an aspect, a stool sample does not include an antibiotic resistant population. For example, a composition can comprise a preparation of viable flora which in proportional content can resemble normal healthy human fecal flora which does not include antibiotic resistant populations. In another aspect, a fecal microbiota in a therapeutic composition is derived from a synthetic fecal composition. In an aspect, a synthetic fecal composition comprises a preparation of viable flora which preferably in proportional content, resembles normal healthy human fecal flora which does not include antibiotic resistant populations. Suitable microorganisms may be selected from the following: Bacteroides, Eubacterium, Fusobacterium, Propionibacterium, Lactobacillus, Ruminococcus, Escherichia coli, Gemmiger, Clostridium, Desulfomonas, Peptostreptococcus, Bifidobacterium, Collinsella, Coprococcus, Dorea, and Ruminococcus.

In an aspect, a preparation of uncultured fecal bacteria extracted from stool of a donor comprises all (100%) of the bacterial strains originally present in the stool of the donor. In an aspect, a preparation of uncultured fecal bacteria extracted from stool of a donor comprises 99.9% of the bacterial strains originally present in the stool of the donor. In an aspect, a preparation of uncultured fecal bacteria extracted from stool of a donor comprises 99.8, 99.7, 99.6, 99.5, 99.4, 99.3, 99.2, 99.1, 99, 98, 97, 96, 95, 94, 93, 92, 91, 90, 89, 88, 87, 85, 84, 83, 82, 81, 80, 75, 70, 65, 60, 55, 50, 45, or 40% of the bacterial strains originally present in the stool of the donor. In one aspect, a pharmaceutical composition comprises reconstituted fecal flora consisting essentially of a combination of a purified fecal microbiota (e.g., a preparation of uncultured fecal bacteria) and a non-cellular fecal filtrate. In another aspect, a pharmaceutical composition comprises a purified fecal microbiota (e.g., a preparation of uncultured fecal bacteria) supplemented with one or more non-cellular non-particulate fecal components. In one aspect, a pharmaceutical composition comprises one or more non-cellular non-particulate fecal components. In an aspect, a therapeutic composition used in a treatment disclosed herein comprises a sterile fecal filtrate or a non-cellular fecal filtrate. In one aspect, a sterile fecal filtrate originates from a donor stool. In another aspect, a sterile fecal filtrate originates from cultured microorganisms. In another aspect, a sterile fecal filtrate comprises a non-cellular non-particulate fecal component. In one aspect, a sterile fecal filtrate is made as described in WO2014/078911, published May 30, 2014. In another aspect, a sterile fecal filtrate is made as described in Ott et al., Gastroenterology 152:799-911(2017). In one aspect, a fecal filtrate comprises secreted, excreted or otherwise liquid components or a microbiota, e.g., biologically active molecules (BAMs), which can be antibiotics or anti-inflammatories, are preserved, retained or reconstituted in a flora extract.

In one aspect, an exemplary therapeutic composition comprises starting material from a donor from a defined donor pool, where this donor contributes a stool that is centrifuged, then filtered with very high-level filtration using e.g., either metal sieving or Millipore filters, or equivalent, to ultimately permit only cells of bacterial origin to remain, e.g., often less than about 5 micrometers diameter. After the initial centrifugation, the solid material is separated from the liquid, and the solid is then filtered in progressively reducing size filters and tangential filters, e.g., using a Millipore filtration, and optionally, also comprising use of nano-membrane filtering. The filtering can also be done by sieves as described in WO 2012/122478, but in contrast using sieves that are smaller than 0.0120 mm, down to about 0.0110 mm, which ultimately result in having only bacterial cells present.

The supernatant separated during centrifugation is now taken and filtered progressively in a filtering, e.g., a Millipore filtering or equivalent systems, to end up with liquid which is finely filtered through an about 0.22 micron filter. This removes all particulate matter including all living matter, including bacteria and viruses. The product then is sterile, but the aim is to remove the bacteria but to keep their secretions, especially antimicrobial bacteriocins, bacteria-derived cytokine-like products and all accompanying Biologically Active Molecules (BAMs), including: thuricin (which is secreted by bacilli in donor stools), bacteriocins (including colicin, troudulixine or putaindicine, or microcin or subtilosin A), lanbiotics (including nisin, subtilin, epidermin, mutacin, mersacidin, actagardine, cinnamycin), lacticins and other antimicrobial or anti-inflammatory compounds.

In one aspect, a therapeutic composition used here comprises a reconstituted fecal flora consisting essentially of a combination of a purified fecal microbiota and a non-cellular fecal filtrate. In another aspect, a therapeutic composition used here comprises a purified fecal microbiota supplemented with one or more non-cellular non-particulate fecal components. In one aspect, a therapeutic composition used here comprises one or more non-cellular non-particulate fecal components. In one aspect, one or more non-cellular non-particulate fecal components comprise synthetic molecules, biologically active molecules produced by a fecal microorganism, or both. In another aspect, one or more non-cellular non-particulate fecal components comprise biologically active proteins or peptides, micronutrients, fats, sugars, small carbohydrates, trace elements, mineral salts, ash, mucous, amino acids, nutrients, vitamins, minerals, or any combination thereof. In one aspect, one or more non-cellular non-particulate fecal components comprise one or more biologically active molecules selected from the group consisting of bacteriocin, lanbiotic, and lacticin. In another aspect, one or more non-cellular non-particulate fecal components comprise one or more bacteriocins selected from the group consisting of colicin, troudulixine, putaindicine, microcin, and subtilosin A. In one aspect, one or more non-cellular non-particulate fecal components comprise one or more lanbiotics selected from the group consisting of thuricin, nisin, subtilin, epidermin, mutacin, mersacidin, actagardine, and cinnamycin. In another aspect, one or more non-cellular non-particulate fecal components comprise an anti-spore compound, an antimicrobial compound, an anti-inflammatory compound, or any combination thereof. In a further aspect, one or more non-cellular non-particulate fecal components comprise an interleukin, a cytokine, a leukotriene, an eicosanoid, or any combination thereof.

In another aspect, a treatment method provided here comprises the use of both fecal bacterial cells, e.g., a partial or a complete representation of the human GI microbiota, and an isolated, processed, filtered, concentrated, reconstituted and/or artificial liquid component (e.g., fecal filtrate) of the flora (the microbiota) which comprises, among others ingredients, bacterial secretory products such as e.g., bacteriocins (proteinaceous toxins produced by bacteria, including colicin, troudulixine or putaindicine, or microcin or subtilosin A), lanbiotics (a class of peptide antibiotics that contain a characteristic polycyclic thioether amino acid lanthionine or methyllanthionine, and unsaturated amino acids dehydroalanine and 2-aminoisobutyric acid; which include thuricin (which is secreted by bacilli in donor stools), nisin, subtilin, epidermin, mutacin, mersacidin, actagardine, cinnamycin), a lacticin (a family of pore-forming peptidic toxins) and other antimicrobial or anti-inflammatory compounds and/or additional biologically active molecules (BAMs) produced by bacteria or other microorganisms of the microbiota, and/or which are found in the “liquid component” of a microbiota.

In one aspect, a fecal bacteria-based therapeutic composition is used concurrently with a fecal non-cellular filtrate-based therapeutic composition. In another aspect, a patient is treated with a first fecal non-cellular filtrate-based therapeutic composition before being given a second fecal bacteria-based therapeutic composition, or vice versa. In a further aspect, a treatment method comprises three steps: first, antibiotic pretreatment to non-selectively remove infectious pathogen(s); second, a fecal non-cellular filtrate-based treatment step to further suppress selected infectious pathogen(s); and third, giving the patient a fecal bacteria-based therapeutic composition to re-establish a functional intestinal microbiome.

In an aspect, a therapeutic composition is combined with other adjuvants such as antacids to dampen bacterial inactivation in the stomach. (e.g., Mylanta, Mucaine, Gastrogel). In another aspect, acid secretion in the stomach could also be pharmacologically suppressed using H2-antagonists or proton pump inhibitors. An example H2-antagonist is ranitidine. An example proton pump inhibitor is omeprazole. In one aspect, an acid suppressant is administered prior to administering, or in co-administration with, a therapeutic composition.

In an aspect, a therapeutic composition is in the form of: an enema composition which can be reconstituted with an appropriate diluent; enteric-coated capsules; enteric-coated microcapsules; acid-resistant tablet; acid-resistant capsules; acid-resistant microcapsules; powder for reconstitution with an appropriate diluent for naso-enteric infusion or colonoscopic infusion; powder for reconstitution with appropriate diluent, flavoring and gastric acid suppression agent for oral ingestion; powder for reconstitution with food or drink; or food or food supplement comprising enteric-coated and/or acid-resistant microcapsules of the composition, powder, jelly, or liquid.

In an aspect, a treatment method effects a cure, reduction of the symptoms, or a percentage reduction of symptoms of an intestinal dysbiosis of a subject. The change of flora is preferably as “near-complete” as possible and the flora is replaced by viable organisms which will crowd out any remaining, original flora. Typically the change in enteric flora comprises introduction of an array of predetermined flora into the gastro-intestinal system, and thus in a preferred form the method of treatment comprises substantially or completely displacing pathogenic enteric flora in patients requiring such treatment.

In another aspect, a therapeutic composition can be provided together with a pharmaceutically acceptable carrier. As used herein, a “pharmaceutically acceptable carrier” refers to a non-toxic solvent, dispersant, excipient, adjuvant, or other material which is mixed with a live bacterium in order to permit the formation of a pharmaceutical composition, e.g., a dosage form capable of administration to the patient. A pharmaceutically acceptable carrier can be liquid (e.g., saline), gel or solid form of diluents, adjuvant, excipients or an acid resistant encapsulated ingredient. Suitable diluents and excipients include pharmaceutical grades of physiological saline, dextrose, glycerol, mannitol, lactose, starch, magnesium stearate, sodium saccharin, cellulose, magnesium carbonate, and the like, and combinations thereof. In another aspect, a therapeutic composition may contain auxiliary substances such as wetting or emulsifying agents, stabilizing or pH buffering agents. In an aspect, a therapeutic composition contains about 1%-5%, 5%-10%, 10%-15%, 15-20%, 20%-25%, 25-30%, 30-35%, 40-45%, 50%-55%, 1%-95%, 2%-95%, 5%-95%, 10%-95%, 15%-95%, 20%-95%, 25%-95%, 30%-95%, 35%-95%, 40%-95%, 45%-95%, 50%-95%, 55%-95%, 60%-95%, 65%-95%, 70%-95%, 45%-95%, 80%-95%, or 85%-95% of active ingredient. In an aspect, a therapeutic composition contains about 2%-70%, 5%-60%, 10%-50%, 15%-40%, 20%-30%, 25%-60%, 30%-60%, or 35%-60% of active ingredient.

In an aspect, a therapeutic composition can be incorporated into tablets, drenches, boluses, capsules or premixes. Formulation of these active ingredients into such dosage forms can be accomplished by means of methods well known in the pharmaceutical formulation arts. See, e.g., U.S. Pat. No. 4,394,377. Filling gelatin capsules with any desired form of the active ingredients readily produces capsules. If desired, these materials can be diluted with an inert powdered diluent, such as sugar, starch, powdered milk, purified crystalline cellulose, or the like to increase the volume for convenience of filling capsules.

In an aspect, conventional formulation processes can be used to prepare tablets containing a therapeutic composition. In addition to the active ingredients, tablets may contain a base, a disintegrator, an absorbent, a binder, and a lubricant. Typical bases include lactose, sugar, sodium chloride, starch and mannitol. Starch is also a good disintegrator as is alginic acid. Surface-active agents such as sodium lauryl sulfate and dioctyl sodium sulphosuccinate are also sometimes used. Commonly used absorbents include starch and lactose. Magnesium carbonate is also useful for oily substances. As a binder there can be used, for example, gelatin, gums, starch, dextrin, polyvinyl pyrrolidone and various cellulose derivatives. Among the commonly used lubricants are magnesium stearate, talc, paraffin wax, various metallic soaps, and polyethylene glycol.

In an aspect, for preparing solid compositions such as tablets, an active ingredient is mixed with a pharmaceutical carrier, e.g., conventional tableting ingredients such as corn starch, lactose, sucrose, sorbitol, talc, stearic acid, magnesium stearate, dicalcium phosphate or gums, or other pharmaceutical diluents, e.g. water, to form a solid preformulation composition containing a homogeneous mixture of a composition of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing a desired amount of an active ingredient (e.g., at least about 10⁵, 10⁶, 10⁷, 10⁸, 10⁹, 10¹⁰, 10¹¹, 10¹², or 10¹³ cfu). A therapeutic composition used herein can be flavored.

In an aspect, a therapeutic composition can be a tablet or a pill. In one aspect, a tablet or a pill can be coated or otherwise compounded to provide a dosage form affording the advantage of prolonged action. For example, a tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

In an aspect, a therapeutic composition can be a drench. In one aspect, a drench is prepared by choosing a saline-suspended form of a therapeutic composition. A water-soluble form of one ingredient can be used in conjunction with a water-insoluble form of the other by preparing a suspension of one with an aqueous solution of the other. Water-insoluble forms of either active ingredient may be prepared as a suspension or in some physiologically acceptable solvent such as polyethylene glycol. Suspensions of water-insoluble forms of either active ingredient can be prepared in oils such as peanut, corn, sesame oil or the like; in a glycol such as propylene glycol or a polyethylene glycol; or in water depending on the solubility of a particular active ingredient. Suitable physiologically acceptable adjuvants may be necessary in order to keep the active ingredients suspended. Adjuvants can include and be chosen from among the thickeners, such as carboxymethylcellulose, polyvinyl pyrrolidone, gelatin and the alginates. Surfactants generally will serve to suspend the active ingredients, particularly the fat-soluble propionate-enhancing compounds. Most useful for making suspensions in liquid nonsolvents are alkylphenol polyethylene oxide adducts, naphthalenesulfonates, alkylbenzene-sulfonates, and the polyoxyethylene sorbitan esters. In addition many substances, which affect the hydrophilicity, density and surface tension of the liquid, can assist in making suspensions in individual cases. For example, silicone anti-foams, glycols, sorbitol, and sugars can be useful suspending agents.

In an aspect, a therapeutic composition comprises non-pathogenic spores of one or more, two or more, three or more, or four or more Clostridium species selected from the group consisting of Clostridium absonum, Clostridium argentinense, Clostridium baratii, Clostridium botulinum, Clostridium cadaveris, Clostridium carnis, Clostridium celatum, Clostridium chauvoei, Clostridium clostridioforme, Clostridium cochlearium, Clostridium fallax, Clostridium felsineum, Clostridium ghonii, Clostridium glycolicum, Clostridium haemolyticum, Clostridium hastiforme, Clostridium histolyticum, Clostridium indolis, Clostridium irregulare, Clostridium limosum, Clostridium malenominatum, Clostridium novyi, Clostridium oroticum, Clostridium paraputrificum, Clostridium perfringens, Clostridium piliforme, Clostridium putrefaciens, Clostridium putrificum, Clostridium sardiniense, Clostridium sartagoforme, Clostridium scindens, Clostridium septicum, Clostridium sordellii, Clostridium sphenoides, Clostridium spiroforme, Clostridium sporogenes, Clostridium subterminale, Clostridium symbiosum, Clostridium tertium, Clostridium tetani, Clostridium welchii, and Clostridium villosum.

In an aspect, a therapeutic composition comprises purified, isolated, or cultured viable non-pathogenic Clostridium and a plurality of purified, isolated, or cultured viable non-pathogenic microorganisms from one or more genera selected from the group consisting of Collinsella, Coprococcus, Dorea, Eubacterium, and Ruminococcus. In another aspect, a therapeutic composition comprises a plurality of purified, isolated, or cultured viable non-pathogenic microorganisms from one or more genera selected from the group consisting of Clostridium, Collinsella, Coprococcus, Dorea, Eubacterium, and Ruminococcus.

In an aspect, a therapeutic composition comprises two or more genera selected from the group consisting of Collinsella, Coprococcus, Dorea, Eubacterium, and Ruminococcus. In another aspect, a therapeutic composition comprises two or more genera selected from the group consisting of Coprococcus, Dorea, Eubacterium, and Ruminococcus. In a further aspect, a therapeutic composition comprises one or more, two or more, three or more, four or more, or five or more species selected from the group consisting of Coprococcus catus, Coprococcus comes, Dorea longicatena, Eubacterium eligens, Eubacterium hadrum, Eubacterium hallii, Eubacterium rectale, and Ruminococcus torques.

In one aspect, a therapeutic composition comprises at least about 10⁵, 10⁶, 10⁷, 10⁸, 10⁹, 10¹⁰, 10¹¹, 10¹², or 10¹³ cfu or total cell count. In another aspect, a therapeutic composition comprises at most about 10⁵, 10⁶, 10⁷, 10⁸, 10⁹, 10¹⁰, 10¹¹, 10¹², 10¹³ or 10¹⁴ cfu or total cell count.

In another aspect, a therapeutic composition comprises at least about 10⁵, 10⁶, 10⁷, 10⁸, 10⁹, 10¹⁰, 10¹¹, 10¹², or 10¹³ cells or total cell count. In another aspect, a therapeutic composition comprises at most about 10⁵, 10⁶, 10⁷, 10⁸, 10⁹, 10¹⁰, 10¹¹, 10¹², 10¹³ or 10¹⁴ cells or total cell count.

In one aspect, a therapeutic composition is formulated as an oral capsule, microcapsule, tablet, or pill. In another aspect, a capsule, microcapsule, tablet, or pill is adapted for enteric delivery. In a further aspect, a capsule, microcapsule, tablet, or pill is an enteric capsule, microcapsule, tablet, or pill. In another aspect, a capsule, microcapsule, tablet, or pill comprises an enteric coating, is acid resistant, or both.

In an aspect, a composition comprising a preparation of uncultured fecal bacteria that is administered to a subject having or at risk for a disorder effects a cure, reduction of the symptoms, or a percentage reduction of symptoms of the disorder based on replacement of bacterial cells endogenous to the intestinal flora of the subject with bacterial cells from the administered bacterial preparation. The change of flora can be as “near-complete” as possible. Typically, the change in enteric flora comprises introduction of an array of flora derived from the stool of a healthy human donor into the gastro-intestinal system of the subject, which can substantially or completely displace pathogenic enteric flora in a patient requiring such treatment (e.g., an IBD or colorectal cancer patient).

The pharmaceutical compositions described herein can comprise microbes, e.g. bacteria, derived from a stool sample of a donor, e.g. a healthy human donor. In an aspect, a composition incorporates uncultured fecal bacteria derived from all or a portion of a fecal microbiota of a stool sample of a healthy human donor. For example, a composition can incorporate a substantially complete fecal microbiota of a stool sample of a healthy human donor. In an aspect, a composition incorporates a bacterial isolate of a fecal microbiota, wherein the bacterial isolate has been purified and/or cultured from all or a portion of the fecal microbiota of a stool sample from a healthy human donor. The harvesting, extraction and/or purification of a fecal microbiota from a stool sample can thus be performed to prepare a composition comprising at least one of uncultured fecal bacteria or a bacterial isolate.

In one aspect, an exemplary therapeutic composition comprises starting material from a donor. In another aspect, an exemplary therapeutic composition comprises material from one or more healthy donors. In yet another aspect, an exemplary therapeutic composition comprises starting material from a defined donor pool. In another aspect, a donor is a healthy subject having an MDI of less than 1. In another aspect, a donor is a healthy subject having an MDI of less than 1.5. In another aspect, a donor is a healthy subject having an MDI of less than 2. In another aspect, a donor is a healthy subject having an MDI of less than 2.5. In another aspect, a donor is a healthy subject having an MDI of less than 3. In another aspect, a donor is an adult male. In a further aspect, a donor is an adult female. In yet another aspect, a donor is an adolescent male. In another aspect, a donor is an adolescent female. In another aspect, a donor is a female toddler. In another aspect, a donor is a male toddler. In another aspect, a donor is healthy. In one aspect, a human donor is a child below about 18, 15, 12, 10, 8, 6, 4, 3, 2, or 1 year old. In another aspect, a human donor is an elderly individual. In a further aspect, a human donor is an individual above about 30, 35, 40, 45, 50, 55, 60, 65, 70, 75, 80, 85, 90, or 95 years old. In another aspect, a donor is about between 1 and 5, between 2 and 10, between 3 and 18, between 21 and 50, between 21 and 40, between 21 and 30, between 50 and 90, between 60 and 90, between 70 and 90, between 60 and 80, or between 65 and 75 years old. In one aspect, a donor is a young old individual (65-74 years). In one aspect, a donor is a middle old individual (75-84 years). In one aspect, a donor is an old individual (>85 years). In yet another aspect, a donor is a carefully screened, healthy, neurotypical human.

In an aspect, a fecal donor can be prescreened for its fecal microbiome profile. In another aspect, a fecal donor can be selected on the basis of the presence of one or more bacterial taxa (e.g., phylum, class, order, family, genus, species or strain) in the donor's stool. In another aspect, a fecal donor can be selected on the basis of the presence of one or more bacterial taxa (e.g., phylum, class, order, family, genus, species or strain) in the donor's stool at a level above a threshold relative abundance.

In an aspect, a carefully screened donor undergoes a complete medical history and physical exam. Donors are excluded if they have a risk of infectious agents. Additional exclusion criteria comprises the following:
1. Known viral infection with Hepatitis B, C or HIV
2. Known exposure to HIV or viral hepatitis at any time
3. High risk behaviors including sex for drugs or money, men who have sex with men, more than one sexual partner in the preceding 12 months, any past use of intravenous drugs or intranasal cocaine, history of incarceration.
4. Tattoo or body piercing within 12 months.
5. Travel to areas of the world where risk of traveler's diarrhea is higher than the US.
6. Current communicable disease, e.g., upper respiratory viral infection.
7. History of irritable bowel syndrome. Specific symptoms may include frequent abdominal cramps, excessive gas, bloating, abdominal distension, fecal urgency, diarrhea, constipation.
8. History of inflammatory bowel disease such as Crohn's disease, ulcerative colitits, microscopic colitis.
9. Chronic diarrhea.
10. Chronic constipation or use of laxatives.
11. History of gastrointestinal malignancy or known colon polyposis.
12. History of any abdominal surgery, e.g., gastric bypass, intestinal resection, appendectomy, cholecystectomy, etc.
13. Use of Probiotics or any other over the counter aids used by the potential donor for purpose of regulating digestion. Yogurt and kefir products are allowed if taken merely as food rather than nutritional supplements.
14. Antibiotics for any indication within the preceding 6 months.
15. Any prescribed immunosuppressive or anti-neoplastic medications.
16. Metabolic Syndrome, established or emerging. Criteria used for definition here are stricter than any established criteria. These include history of increased blood pressure, history of diabetes or glucose intolerance.
17. Known systemic autoimmunity, e.g., connective tissue disease, multiple sclerosis.
18. Known atopic diseases including asthma or eczema.
19. Chronic pain syndromes including fibromyalgia, chronic fatigue syndrome.
20. Ongoing (even if intermittent) use of any prescribed medications, including inhalers or topical creams and ointments.
21. Neurologic, neurodevelopmental, and neurodegenerative disorders including autism, Parkinson's disease.
22. General. Body mass index >26 kg/m2, central obesity defined by waste:hip ratio >0.85 (male) and >0.80 (female).
23. Blood pressure >135 mmHg systolic and >85 mmHg diastolic.
24. Skin—presence of a rash, tattoos or body piercing placed within a year, or jaundice 25. Enlarged lymph nodes.
26. Wheezing on auscultation.
27. Hepatomegaly or stigmata of liver disease.
28. Swollen or tender joints. Muscle weakness.
29. Abnormal neurologic examination.
30. Positive stool Clostridium difficile toxin B tested by PCR.
31. Positive stool cultures for any of the routine pathogens including Salmonella, Shigella, Yersinia, Campylobacter, E. coli 0157:H7.
32. Abnormal ova and parasites examination.
33. Positive Giardia, Cryptosporidium, or Helicobacter pylori antigens.
34. Positive screening for any viral illnesses, including HIV 1 and 2, Viral Hepatitis A IgM, Hepatitis surface antigen and core Ab.
35. Abnormal RPR (screen for syphilis).
36. Any abnormal liver function tests including alkaline phosphatase, aspartate aminotransaminase, alanine aminotransferase.
37. Raised serum triglycerides >150 mg/Dl
38. HDL cholesterol <40 mg/dL (males) and <50 mg/dL (females)
39. High sensitivity CRP >2.4 mg/L
40. Raised fasting plasma glucose (>100 mg/dL)

In one aspect, provided herein is a process for collecting and processing a stool sample to give rise to a preparation of uncultured fecal bacteria. The process can comprise first collecting a stool sample from one or more healthy (e.g., screened) donor(s). In one aspect, a fresh stool is transported via a stool collection device, which can provide or comprises a suitably oxygen free (or substantially oxygen free) appropriate container. In one aspect, the container can be made oxygen free by e.g., incorporating into the container a built in or clipped-on oxygen-scavenging mechanism, e.g., oxygen scavenging pellets as described e.g., in U.S. Pat. No. 7,541,091. In another aspect, the container itself is made of an oxygen scavenging material, e.g., oxygen scavenging iron, e.g., as described by O2BLOCK™, or equivalents, which uses a purified and modified layered clay as a performance-enhancing carrier of oxygen-scavenging iron; the active iron is dispersed directly in the polymer. In one aspect, oxygen-scavenging polymers are used to make the container itself or to coat the container, or as pellets to be added; e.g., as described in U.S. Pat. App. Pub. 20110045222, describing polymer blends having one or more unsaturated olefinic homopolymers or copolymers; one or more polyamide homopolymers or copolymers; one or more polyethylene terephthalate homopolymers or copolymers; that exhibit oxygen-scavenging activity. In one aspect, oxygen-scavenging polymers are used to make the container itself or to coat the container, or as pellets to be added; e.g., as described in U.S. Pat. App. Pub. 20110008554, describing compositions comprising a polyester, a copolyester ether and an oxidation catalyst, wherein the copolyester ether comprises a polyether segment comprising poly(tetramethylene-co-alkylene ether). In one aspect, oxygen-scavenging polymers are used to make the container itself or to coat the container, or as pellets to be added; e.g., as described in U.S. Pat. App. Pub. 201000255231, describing a dispersed iron/salt particle in a polymer matrix, and an oxygen scavenging film with oxygen scavenging particulates.

Alternatively, in addition to or in place of the oxygen-scavenging mechanism, the air in the container can be replaced (completely or substantially) with nitrogen and/or other inert non-reactive gas or gases. In one aspect, the container simulates (creates) partially, substantially or completely an anaerobic environment.

In one aspect, the stool (e.g., fecal sample) is held in an aesthetically acceptable container that will not leak nor smell yet maintain an anaerobic environment. In one aspect, the container is sterile before receiving the fecal flora.

In one aspect, a stool sample provided herein is maintained at room temperature during most or all of its transportation and/or storage at e.g., a “stool bank”. For example, once delivered to a “processing stool bank” it is stored at ambient temperature, e.g., room temperature. In one aspect, stabilizing agents, such as glycerol, are added to the harvested and/or stored material.

In one aspect, the stool is tested for various pathogens, as noted above. In one aspect, once cleared of infective agents, a stool sample is homogenized and filtered to remove large particles of matter. In one aspect, the stool is subdivided into desired volumes, e.g., which can be between 5 cc and 3 or more liters. For example, in one aspect, a container comprises a 50 gram (g) stool, which can be held in an appropriate oxygen resistant plastic, e.g., a metallized polyethylene terephthalate polyester film, or a metallized MYLAR™.

In one aspect, the stool is subject to homogenization by for example, mixing, agitating, stirring or shaking. In certain aspects, a stool sample is diluted with a homogenization buffer prior to homogenization. A homogenization buffer can, for example, contain a cryoprotectant (e.g., trehalose), an antioxidant or reducing agent (e.g., cysteine), and a buffer (e.g., 0.25×PBS at pH 7.4).

In one aspect, to separate the non-bacterial components from the fecal microbiota, the stool can be homogenized and filtered from rough particulate matter. In one aspect, the microscopic fiber/nonliving matter is then separated from the bacteria. Several methods can be used, including e.g., recurrent filtration with filter sizes, e.g., progressively coming down to the size of a typical bacterium.

In one aspect, different filters are used to isolate bacterial sp., or a technique as used by Williams in WO 2011/033310A1, which uses a crude technique of filtration with a gauze.

In one aspect, a filtration procedure for filtering whole stool is suitably used to reach the highest concentration of almost 100% bacteria. In one aspect, the filtering procedure is a two-step procedure suitably using glass fibre depth filters for initial clarification. In one aspect, the stool is filtered under positive pressure. In one aspect, this would be using a combination or sandwich configuration with a 30 micron PVDF filter. In one aspect, this sandwich procedure will be filtering the product under positive pressure. Later, membrane concentration can, in one aspect, be used as another step to reduce the volume of the filtrate. In one aspect, this can be done prior to freeze drying or spray drying under nitrogen cover.

Alternative membranes that can be used for filtration include, but not limited to, nylon filters, cellulose nitrate filters, polyethersulfone (PES) filters, polytetrafluorethylene (PTFE) filters, TEFLON™ filters, mixed cellulose Ester filters, polycarbonate filters, polypropylene filters, Polyvinylchloride (PVC) filters or quartz filters. Various combinations of these can be used to achieve a high purity of bacteria with solids and liquid removed.

In another aspect, a pharmaceutical composition comprises a bacterial mixture comprising a preparation of uncultured fecal bacteria supplemented, spiked, enriched, or enhanced with one or more bacterial isolates (e.g., a probiotic). For example, a bacterial mixture can comprise a preparation of uncultured fecal bacteria spiked with one or more bacterial isolates comprising one or more bacterial strains. By enriching or spiking a preparation of uncultured fecal bacteria derived from a stool sample (e.g., a fecal microbiota) of a healthy donor with one or more non-pathogenic bacterial isolates, a bacterial mixture can be produced in which the amount of a particular bacterial strain or strains (i.e. the spiked-in bacterial isolate(s)) can be accounted for and precisely controlled. Without wishing to be bound by theory, this is advantageous, for example, where a bacterial isolate mixed with the preparation of uncultured fecal bacteria is important for or involved in the treatment of a disorder of a subject, but insufficient on its own to generate a complete or optimal treatment response in the subject. Unlike probiotics, administration to a subject of one or more bacterial isolates together with a preparation of uncultured fecal bacteria (i.e., derived from a healthy donor) provides the subject with the advantage of the administered bacterial isolate combined with multi-factorial benefits conferred by the additional fecal bacterial strains present in the uncultured preparation. These additional fecal bacterial strains may combine to, for example, provide for the necessary context or interactions (e.g. via one or more released factors) to enable the bacterial isolate to induce an optimal response in the subject, or may directly induce a response in the subject that combines and/or synergizes with a response induced by the bacterial isolate to treat the subject. Accordingly, in certain aspects, a pharmaceutical composition comprising a mixture of one or more bacterial isolates and a preparation of uncultured fecal bacteria can be more effective in treating a disorder of a subject than a composition comprising either the bacterial isolate or the preparation of uncultured fecal bacteria alone.

Pharmaceutical Compositions, Formulations, and Administration

Described herein are pharmaceutical compositions comprising a bacterial mixture comprising a preparation of uncultured fecal bacteria in various formulations. Any pharmaceutical composition (and/or additional therapeutic agents) described herein can take the form of tablets, pills, pellets, capsules, capsules containing liquids, capsules containing multiparticulates, powders, solutions, emulsion, drops, suppositories, emulsions, aerosols, sprays, suspensions, delayed-release formulations, sustained-release formulations, controlled-release formulations, or any other form suitable for use.

The formulations comprising the pharmaceutical compositions described herein can conveniently be presented in unit dosage forms. For example, the dosage forms can be prepared by methods which include the step of bringing the therapeutic agents into association with a carrier, which constitutes one or more accessory ingredients. For example, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by press tableting).

In another aspect, a pharmaceutical composition can include a pharmaceutically acceptable carrier. As used herein, a “pharmaceutically acceptable carrier” refers to a non-toxic solvent, dispersant, excipient, adjuvant, or other material which is mixed with a live bacterium in order to permit the formation of a pharmaceutical composition, e.g., a dosage form capable of administration to the patient. A pharmaceutically acceptable carrier can be liquid (e.g., saline), gel or solid form of diluents, adjuvant, excipients or an acid resistant encapsulated ingredient. Suitable diluents and excipients include pharmaceutical grades of physiological saline, dextrose, glycerol, mannitol, lactose, starch, magnesium stearate, sodium saccharin, cellulose, magnesium carbonate, and the like, and a combination thereof. In another aspect, a pharmaceutical composition can contain auxiliary substances such as wetting or emulsifying agents, stabilizing or pH buffering agents. In an aspect, a pharmaceutical composition contains about 1%-5%, 5%-10%, 10%-15%, 15-20%, 20%-25%, 25-30%, 30-35%, 40-45%, 50%-55%, 1%-95%, 2%-95%, 5%-95%, 10%-95%, 15%-95%, 20%-95%, 25%-95%, 30%-95%, 35%-95%, 40%-95%, 45%-95%, 50%-95%, 55%-95%, 60%-95%, 65%-95%, 70%-95%, 45%-95%, 80%-95%, or 85%-95% of active ingredient. In an aspect, a pharmaceutical composition contains about 2%-70%, 5%-60%, 10%-50%, 15%-40%, 20%-30%, 25%-60%, 30%-60%, or 35%-60% of active ingredient.

In an aspect, a pharmaceutical composition can include or be incorporated into tablets, drenches, boluses, capsules or premixes. Formulation of these active ingredients into such dosage forms can be accomplished by means of methods well known in the pharmaceutical formulation arts. See, e.g., U.S. Pat. No. 4,394,377. Filling gelatin capsules with any desired form of the active ingredients readily produces capsules. If desired, these materials can be diluted with an inert powdered diluent, such as sugar, starch, powdered milk, purified crystalline cellulose, or the like to increase the volume for convenience of filling capsules.

In an aspect, for preparing solid compositions such as tablets, an active ingredient is mixed with a pharmaceutical carrier, e.g., conventional tableting ingredients such as cornstarch, lactose, sucrose, sorbitol, talc, stearic acid, magnesium stearate, dicalcium phosphate or gums, or other pharmaceutical diluents, e.g. water, to form a solid preformulation composition containing a homogeneous mixture of a composition described herein. When referring to these preformulation compositions as homogeneous, it is meant that the active ingredient is dispersed evenly throughout the composition so that the composition can be readily subdivided into equally effective unit dosage forms such as tablets, pills and capsules. This solid preformulation composition is then subdivided into unit dosage forms of the type described above containing a desired amount of an active ingredient. A pharmaceutical composition described herein can be flavored.

In an aspect, a pharmaceutical composition comprising a bacterial mixture described herein (and optionally one or more additional therapeutic agents) is formulated as a composition adapted for a mode of administration described herein.

In various aspects, the administration of the pharmaceutical compositions is any one of oral, intravenous, intraperitoneal, and parenteral. For example, routes of administration include, but are not limited to, oral, intraperitoneal, intravenous, intramuscular, or rectal. In various aspects, the administration of the pharmaceutical compositions is oral, nasogastric, antegrade gastrointestinal, retrograde gastrointestinal, endoscopic, or enemic.

In an aspect, a pharmaceutical composition described herein can be formulated as a composition adapted for oral administration. Compositions for oral delivery can be in the form of tablets, lozenges, aqueous or oily suspensions, granules, powders, sprinkles, emulsions, capsules, syrups, or elixirs, for example. Orally administered compositions can comprise one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration to provide sustained delivery of the bacterial mixture over an extended period of time. Selectively permeable membranes surrounding an osmotically active agent are also suitable for orally administered compositions. In these latter platforms, fluid from the environment surrounding the capsule is imbibed by a driving compound, which swells to displace the agent or agent composition through an aperture. These delivery platforms can provide an essentially zero order delivery profile as opposed to the spiked profiles of immediate release formulations. A time-delay material, such as glycerol monostearate or glycerol stearate, can also be useful. Oral compositions can include standard excipients such as mannitol, lactose, starch, magnesium stearate, sodium saccharin, cellulose, ethacrylic acid and derivative polymers thereof, and magnesium carbonate. In an aspect, the excipients are of pharmaceutical grade. Suspensions, in addition to the active compounds, can contain suspending agents such as, for example, ethoxylated isostearyl alcohols, polyoxyethylene sorbitol and sorbitan esters, microcrystalline cellulose, aluminum metahydroxide, bentonite, agar-agar, tragacanth, etc., and mixtures thereof.

In various aspects, a pharmaceutical composition is formulated as a solid dosage form such as a tablets, dispersible powder, granule, or capsule. In an aspect, the pharmaceutical composition is formulated as a capsule. In another aspect, the pharmaceutical composition is formulated as a tablet. In yet another aspect, the pharmaceutical composition is formulated as a softgel capsule. In a further aspect, the pharmaceutical composition is formulated as a gelatin capsule.

In an aspect, a pharmaceutical composition is in the form of: an enema composition which can be reconstituted with an appropriate diluent; an enteric-coated capsule; an enteric-coated microcapsule; an acid-resistant tablet; an acid-resistant capsules; an acid-resistant microcapsule; powder for reconstitution with an appropriate diluent for naso-enteric infusion or colonoscopic infusion; powder for reconstitution with appropriate diluent, flavoring and gastric acid suppression agent for oral ingestion; powder for reconstitution with food or drink; or food or food supplement comprising enteric-coated and/or acid-resistant microcapsules of the composition, powder, jelly, or liquid.

In various aspects, formulations can additionally comprise a pharmaceutically acceptable carrier or excipient. As one skilled in the art will recognize, the formulations can be in any suitable form appropriate for the desired use and route of administration.

In some dosage forms, a pharmaceutical composition described herein is mixed with at least one inert, pharmaceutically acceptable excipient or carrier such as sodium citrate, dicalcium phosphate, etc., and/or a) fillers or extenders such as starches, lactose, sucrose, glucose, mannitol, silicic acid, microcrystalline cellulose, and Bakers Special Sugar, etc., b) binders such as, for example, carboxymethylcellulose, alginates, gelatin, polyvinylpyrrolidone, sucrose, acacia, polyvinyl alcohol, polyvinylpyrrolidone, methylcellulose, hydroxypropyl cellulose (HPC), and hydroxymethyl cellulose etc., c) humectants such as glycerol, etc., d) disintegrating agents such as agar-agar, calcium carbonate, potato or tapioca starch, alginic acid, certain silicates, sodium carbonate, cross-linked polymers such as crospovidone (cross-linked polyvinylpyrrolidone), croscarmellose sodium (cross-linked sodium carboxymethylcellulose), sodium starch glycolate, etc., e) solution retarding agents such as paraffin, etc., f) absorption accelerators such as quaternary ammonium compounds, etc., g) wetting agents such as, for example, cetyl alcohol and glycerol monostearate, etc., h) absorbents such as kaolin and bentonite clay, etc., and i) lubricants such as talc, calcium stearate, magnesium stearate, solid polyethylene glycols, sodium lauryl sulfate, glyceryl behenate, etc., and mixtures of such excipients. One of skill in the art will recognize that particular excipients can have two or more functions in the oral dosage form. In the case of an oral dosage form, for example, a capsule or a tablet, the dosage form can also comprise buffering agents.

In an aspect, a pharmaceutical composition comprising a bacterial mixture is combined with one or more pharmaceutically acceptable cryoprotectants, lyoprotectants, binders, disintegrants, excipients, fillers, and/or preservatives, acid suppressants, antacids, H2 antagonists, and proton pump inhibitors, or combinations thereof.

In an aspect, a pharmaceutical composition comprising a bacterial mixture is combined with other adjuvants such as antacids to dampen bacterial inactivation in the stomach. (e.g., Mylanta, Mucaine, Gastrogel). In another aspect, acid secretion in the stomach could also be pharmacologically suppressed using H2-antagonists or proton pump inhibitors. An example H2-antagonist is ranitidine. An example proton pump inhibitor is omeprazole. In one aspect, an acid suppressant is administered prior to administering, or in co-administration with, a pharmaceutical composition.

In one aspect, a pharmaceutical composition administered herein further comprises an acid suppressant, an antacid, an H2 antagonist, a proton pump inhibitor or a combination thereof. In one aspect, a pharmaceutical composition administered herein is substantially free of non-living matter. In another aspect, a pharmaceutical composition administered herein substantially free of acellular material selected from the group consisting of residual fiber, DNA, viral coat material, and non-viable material. In another aspect, a pharmaceutical composition administered does not comprise an acid suppressant, an antacid, an H2 antagonist, a proton pump inhibitor or a combination thereof. In yet another aspect, a pharmaceutical composition administered does not comprise an acid suppressant. In another aspect, a pharmaceutical composition administered does not comprise an antacid. In another aspect, a pharmaceutical composition administered does not comprise an H2 antagonist. In another aspect, a pharmaceutical composition administered does not comprise a proton pump inhibitor. In another aspect, a pharmaceutical composition administered does not comprise metoclopramide.

In an aspect, a bacterial mixture is dry, e.g., when it includes lyophilized bacterial cells/spores or comprises dry binders, fillers, and dispersants. Alternately, the bacterial mixture can be aqueous, e.g., when it comprises non-dry binders, fillers, and dispersants.

In an aspect, a bacterial mixture described herein can be subject to lyophilization. As used herein, “lyophilization” or “freeze drying” refers to the process of drying a material by first freezing it and then encouraging the ice within it to sublimate in a vacuum environment.

In one aspect, a bacterial mixture comprises a lyophilized formulation further comprising a reducing agent and/or antioxidant. In certain aspects, the reducing agent comprises cysteine selected from the group consisting of D-cysteine and L-cysteine. In another aspect, cysteine is at a concentration of at least about 0.025%. In one aspect, cysteine is at a concentration of about 0.025%. In another aspect, cysteine is at a concentration of 0.025%. In another aspect, another reducing agent other than cysteine is used in lieu of, or in combination with cysteine. In an aspect, another reducing agent is selected from the group comprising ascorbic acid, sodium ascorbate, thioglycolic acid, sodium sulfite, sodium bisulfite, sodium metabisulfite, potassium metabisulfite, glutathione, methionine, thioglycerol, and alpha tocopherol.

In one aspect, a bacterial mixture comprises a cryoprotectant or mixture of cryoprotectants. As used herein, a “cryoprotectant” refers to a substance that is added to a formulation in order to protect an active ingredient during freezing. For example, a cryoprotectant can comprise, consist essentially of, or consist of polyethylene glycol, skim milk, erythritol, arabitol, sorbitol, glucose, fructose, alanine, glycine, proline, sucrose, lactose, ribose, trehalose, dimethyl sulfoxide (DMSO) or equivalent, a glycerol, a polyethylene glycol (PEG) or equivalent, or an amino acid (e.g., alanine, glycine, proline). In an aspect of the present disclosure, a cryoprotectant can be selected from the group comprising 5% Sucrose; 10% Sucrose; 10% Skim milk; 10% Trehalose with 2.5% sucrose; 5% Trehalose with 2.5% sucrose; 5% Mannitol; 5% Mannitol with 0.1% Polysorbate 80; 10% Mannitol; 10% Mannitol with 0.1% Polysorbate 80; 5% Trehalose; 5% Trehalose with 0.1% Polysorbate 80; 10% Trehalose; and 10% Trehalose with 0.1% Polysorbate 80.

In an aspect, a bacterial mixture comprises a lyoprotectant. As used herein, a “lyoprotectant” refers to a substance that is added to a formulation in order to protect an active ingredient during lyophilization. In one aspect, the same substance or the same substance combination is used as both a cryoprotectant and a lyoprotectant. Exemplary lyoprotectants include sugars such as sucrose or trehalose; an amino acid such as monosodium glutamate or histidine; a methylamine such as betaine; a lyotropic salt such as magnesium sulfate; a polyol such as trihydric or higher sugar alcohols, e.g. glycerin, erythritol, glycerol, arabitol, xylitol, sorbitol, and mannitol; propylene glycol; polyethylene glycol; Pluronics; and a combination thereof. In an aspect, a lyoprotectant is a non-reducing sugar, such as trehalose or sucrose. In an aspect, a cryoprotectant or a lyoprotectant consists essentially of, or consists of, one or more substances mentioned in this paragraph and the paragraph above.

In one aspect, a subject in need thereof is administered a therapeutic composition comprising fecal microbiota of multiple carefully screened, healthy donors. In an aspect, a subject is administered a therapeutic composition over a dosing period wherein a first dose comprises at least one therapeutic composition comprises fecal microbiota of a single donor, and a second dose of a therapeutic composition comprises fecal microbiota of a single donor different from the donor of the first dose. In another aspect, a first dose comprises a therapeutic composition comprising fecal microbiota of a single donor and a second dose comprises fecal microbiota of a donor pool. The first and second dose do not indicate the order of administration to a subject, but rather that fecal microbiota from separate donors may be used in a non-blended form.

In another aspect, the present disclosure provides for methods for treating a subject in need thereof with capsules containing a therapeutic composition comprising fecal microbiota from a single donor. In another aspect, a capsule comprises a therapeutic composition comprising fecal microbiota from multiple donors. In one aspect a subject is administered two or more pills comprising fecal microbiota from a single but different donor.

In one aspect, the present disclosure provides for methods for treating a subject in need thereof comprising administering a therapeutic composition orally or by infusions through a colonoscope, an enema or via a nasojejunal tube. In another aspect, each administration comprises a therapeutic composition comprising fecal microbiota of a single donor similar to or different from a prior administration in a treatment period. In another aspect, a treatment period includes administration of a first dost comprising a therapeutic composition comprising fecal microbiota of a single donor and administration of a second dose comprising a therapeutic composition comprising fecal microbiota of multiple donors.

Additional Therapeutic Agents and Co-Formulation

The pharmaceutical compositions described herein can include one or more therapeutic agents in addition to a bacterial mixture, which can be administered to a subject in need thereof in a method described herein. The additional therapeutic agent can be administered simultaneous or sequential with a bacterial mixture (e.g., comprising one or more bacterial isolates and/or a preparation of uncultured fecal bacteria) described herein. Further, the present compositions and formulations can comprise the additional therapeutic agent (e.g. via co-formulation). For example, the additional therapeutic agent, one or more bacterial isolates, and preparation of uncultured fecal bacteria can be combined into a single formulation.

In an aspect, the additional therapeutic agent and bacterial mixture are administered to a subject simultaneously. The term “simultaneously” as used herein, means that the additional therapeutic agent and the bacterial mixture are administered with a time separation of no more than about 60 minutes, such as no more than about 30 minutes, no more than about 20 minutes, no more than about 10 minutes, no more than about 5 minutes, or no more than about 1 minute. Administration of the additional therapeutic agent and the bacterial mixture can be by simultaneous administration of a single formulation (e.g., a formulation comprising the additional therapeutic agent and a bacterial mixture) or of separate formulations (e.g., a first formulation including the additional therapeutic agent and a second formulation including the bacterial mixture).

Co-administration does not require an additional therapeutic agent to be administered simultaneously, if the timing of its administration is such that the pharmacological activities of the additional therapeutic agent and the bacterial mixture (e.g., comprising one or more bacterial isolates and/or a preparation of uncultured fecal bacteria) overlap in time. For example, the additional therapeutic agent and the bacterial mixture can be administered sequentially. The term “sequentially” as used herein means that the additional therapeutic agent and the bacterial mixture are administered with a time separation of more than about 60 minutes. For example, the time between the sequential administration of the additional therapeutic agent and the bacterial mixture can be more than about 60 minutes, more than about 2 hours, more than about 5 hours, more than about 10 hours, more than about 1 day, more than about 2 days, more than about 3 days, or more than about 1 week apart. The optimal administration times will depend on the rates of metabolism, excretion, and/or the pharmacodynamic activity of the additional therapeutic agent and the bacterial mixture being administered. Either of the additional therapeutic agent or the bacterial mixture can be administered first.

In a further aspect, the additional therapeutic agent and the bacterial mixture can be administered to a subject simultaneously but the release of additional therapeutic agent and the bacterial mixture from their respective dosage forms (or single unit dosage form if co-formulated) in the GI tract can occur sequentially.

Co-administration also does not require multiple additional therapeutic agents to be administered to the subject by the same route of administration as a bacterial mixture. Rather, each additional therapeutic agent can be administered by any appropriate route, for example, parenterally or non-parenterally.

In some aspects, the additional therapeutic agent is an agent used to treat or prevent one or more symptoms of a disorder described herein. In some aspects, the additional therapeutic agent is selected from the group consisting of isperidone, fluoxetine, aripiprazole, vitamin D, levocarnitine, and a combination thereof.

In some aspects, the additional therapeutic agent is an anti-inflammatory agent such as steroidal anti-inflammatory agents or non-steroidal anti-inflammatory agents (NSAIDS). Steroids, particularly the adrenal corticosteroids and their synthetic analogues, are well known in the art. Non-limiting examples of corticosteroids that can be administered to a subject as an additional therapeutic agent include hydroxyltriamcinolone, alpha-methyl dexamethasone, beta-methyl betamethasone, beclomethasone dipropionate, betamethasone benzoate, betamethasone dipropionate, betamethasone valerate, clobetasol valerate, desonide, desoxymethasone, dexamethasone, diflorasone diacetate, diflucortolone valerate, fluadrenolone, fluclorolone acetonide, flumethasone pivalate, fluosinolone acetonide, fluocinonide, flucortine butylester, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone acetonide, medrysone, amcinafel, amcinafide, betamethasone and the balance of its esters, chloroprednisone, clocortelone, clescinolone, dichlorisone, difluprednate, flucloronide, flunisolide, fluoromethalone, fluperolone, fluprednisolone, hydrocortisone, meprednisone, paramethasone, prednisolone, prednisone, beclomethasone dipropionate. (NSAIDS) that can be used, include but are not limited to, salicylic acid, acetyl salicylic acid, methyl salicylate, glycol salicylate, salicylmides, benzyl-2,5-diacetoxybenzoic acid, ibuprofen, fulindac, naproxen, ketoprofen, etofenamate, phenylbutazone, indomethacin, and a combination thereof. Additional anti-inflammatory agents are described, for example, in U.S. Pat. No. 4,537,776, the entire contents of which is incorporated by reference herein.

In some aspects, an additional therapeutic agent that can be incorporated into a pharmaceutical composition is a prebiotic. A prebiotic is a compound or compounds (e.g. comprising one or more nutrients) administered to a subject to promote the growth, proliferation, or activity of one or more microorganisms (e.g., bacteria) in the intestine of the subject (e.g., by providing a substrate to be metabolized by the one or more microorganisms). Without wishing to be bound by theory, prebiotics can be added to a pharmaceutical composition to nutritionally supplement bacteria in the endogenous microbiome of the subject and/or in the pharmaceutical composition itself, e.g., to stimulate the growth or activity of one or more strains of a preparation of uncultured fecal bacteria and/or one or more bacterial isolates. Additionally, one or more prebiotics can be added to a composition to buffer against “shock” to bacteria cells when transitioning those cells to a new environment, for example, subsequent to the isolation and/or purification of a preparation of uncultured fecal bacteria, or before or after freezing, freeze-drying, spray-drying, reconstitution in solution and the like.

Non-limiting examples of prebiotics that can be added to a pharmaceutical composition include an amino acid (e.g., valine, leucine, isoleucine), lactic acid, ammonium nitrate, amylose, barley mulch, biotin, carbonate, cellulose, chitin, choline, fructooligosaccharides (FOSs), fructose, glucose, glycerol, heteropolysaccharide, histidine, homopolysaccharide, hydroxyapatite, inulin, isomaltulose, lactose, lactulose, maltodextrins, maltose, nitrogen, oligodextrose, oligofructose, oligofructose-enriched inulin, an oligosaccharide (e.g. comprising a galactooligosaccharide (GOS), trans-galactooligosaccharide, fructooligosaccharide (FOS), xylooligosaccharides (XOS), mannooligosaccharide, or chitooligosaccharide), pectin, phosphate salts, phosphorus, polydextroses, polyols, potash, potassium, sodium nitrate, starch, sucrose, sulfur, sun fiber, tagatose, thiamine, trehalose, vitamins, a water-soluble carbohydrate, a fermentable polysaccharide, a dietary fiber, resistant starch, barley, white navy bean powder, and a combination thereof. Illustrative prebiotics include complex carbohydrates, amino acids, peptides, or other essential nutritional components for the survival of the bacterial composition.

In an aspect, a subject is not pretreated with a prebiotic prior to treatment with a pharmaceutical composition. In another aspect, the pharmaceutical composition is not supplemented with a prebiotic.

In an aspect, a prebiotic can be included (e.g., in dry or liquid forms) in a pharmaceutical composition described herein, for example, comprising a bacterial mixture.

Alternately, or additionally, a prebiotic to be administered to a subject can be included (e.g., in dry or liquid forms) in a distinct pharmaceutical composition lacking a bacterial mixture.

A prebiotic can be administered to a subject before, contemporaneously with, and/or after administration of a pharmaceutical composition comprising a bacterial mixture, either in the same pharmaceutical composition or in a separate pharmaceutical composition.

A prebiotic can be provided and administered in a single dose or in multiple doses. When provided as a single dose, a single composition can comprise only one prebiotic or a mixture of prebiotics. When provided in multiple doses, each composition dosed to the subject can comprise a single prebiotic or a mixture of prebiotics, and/or a first composition dosed to the subject can comprise a different prebiotic or prebiotics than a second composition dosed to the subject.

As examples, when multiple doses are provided, a first composition comprising a prebiotic can include a first prebiotic, e.g., inulin, and a second composition can include a different prebiotic, e.g., fructooligosaccharide, with or without the first prebiotic. Alternately, a first composition can include a combination of prebiotics, e.g., inulin and fructooligosaccharide and a second composition can include a different combination of prebiotics, e.g., inulin and white navy bean powder. A first composition can include a combination of prebiotics and a second composition can include only one prebiotic.

The amount of prebiotic included in a composition depends on the specific prebiotic, the specific bacterial strain or strains targeted by the prebiotic, and/or the disease state of the subject/patient.

In some aspects, an additional therapeutic agent be incorporated into a pharmaceutical composition is an antidiarrheal agent. Non-limiting examples of antidiarrheal agents suitable for inclusion in a pharmaceutical composition described herein include, but are not limited to, DPP-IV inhibitors, natural opioids, such as tincture of opium, paregoric, and codeine, synthetic opioids, such as diphenoxylate, difenoxin and loperamide, bismuth subsalicylate, lanreotide, vapreotide and octreotide, motiln antagonists, COX2 inhibitors like celecoxib, glutamine, thalidomide and traditional antidiarrheal remedies, such as kaolin, pectin, berberine and muscarinic agents, and a combination thereof.

In some aspects, the additional therapeutic agent incorporated into a pharmaceutical composition can be an analgesic. Analgesics useful in the compositions and methods described herein include, without limitation, morphine, codeine, heroine, methadone and related compounds, thebaine, orpiavine, and their derivatives, buprenorphine, the piperidines, morphinans, benzomorphans, tetra-hydroisoquinolines, thiambutanes, benzylamines, tilidine, viminol, nefopam, capsaicin(8-methyl-N-vanillyl-6E-nonenamide), “synthetic” capsaicin(N-vanillylnonamide) and related compounds, and a combination thereof.

In some aspects, the additional therapeutic agent is an anti-bacterial agent, which includes, but is not limited to, cephalosporin antibiotics (cephalexin, cefuroxime, cefadroxil, cefazolin, cephalothin, cefaclor, cefamandole, cefoxitin, cefprozil, and ceftobiprole); fluoroquinolone antibiotics (cipro, Levaquin, floxin, tequin, avelox, and norflox); tetracycline antibiotics (tetracycline, minocycline, oxytetracycline, and doxycycline); penicillin antibiotics (amoxicillin, ampicillin, penicillin V, dicloxacillin, carbenicillin, vancomycin, and methicillin); monobactam antibiotics (aztreonam); carbapenem antibiotics (ertapenem, doripenem, imipenem/cilastatin, and meropenem); and a combination thereof. In some aspects, the anti-bacterial agent can be any of the penicillin, cephalosporin, monobactam, and carbapenem antibiotics, or a combination thereof.

In one aspect, a method further comprises pretreating a subject with an antibiotic composition prior to administering a therapeutic bacterial mixture. In one aspect, an antibiotic composition administered herein comprises an antibiotic selected from the group consisting of rifabutin, clarithromycin, clofazimine, vancomycin, rifampicin, nitroimidazole, chloramphenicol, and a combination thereof. In another aspect, an antibiotic composition administered herein comprises an antibiotic selected from the group consisting of rifaximin, rifamycin derivative, rifampicin, rifabutin, rifapentine, rifalazil, bicozamycin, aminoglycoside, gentamycin, neomycin, streptomycin, paromomycin, verdamicin, mutamicin, sisomicin, netilmicin, retymicin, kanamycin, aztreonam, aztreonam macrolide, clarithromycin, dirithromycin, roxithromycin, telithromycin, azithromycin, bismuth subsalicylate, vancomycin, streptomycin, fidaxomicin, amikacin, arbekacin, neomycin, netilmicin, paromomycin, rhodostreptomycin, tobramycin, apramycin, and a combination thereof. In another aspect, a subject is not pretreated with an antibiotic composition prior to administering a bacterial mixture. In another aspect, the pharmaceutical composition is not supplemented with an antibiotic composition. In a further aspect, a method further comprises pretreating a subject with an anti-inflammatory drug prior to administration of a bacterial mixture. In yet another aspect, a subject is not pretreated with an anti-inflammatory drug prior to administering a bacterial or mixture. In another aspect, a bacterial mixture is not supplemented with an anti-inflammatory.

Delivery of an additional therapeutic agent can be targeted to various parts of the GI tract, as described herein.

In an aspect, a preparation of uncultured fecal bacteria and one or more bacterial isolates are administered to a subject according to a method described herein in the same pharmaceutical composition. In an aspect, a preparation of uncultured fecal bacteria and one or more bacterial isolates are administered to a subject according to a method described herein in different pharmaceutical compositions. In an aspect, multiple bacterial isolates are administered to a subject according to a method described herein in the same pharmaceutical composition. In an aspect, multiple bacterial isolates are administered to a subject according to a method described herein in different pharmaceutical compositions. For example, a method can comprise administering to a subject in need thereof an effective amount of a plurality of pharmaceutical compositions, e.g., two or more pharmaceutical compositions, three or more pharmaceutical compositions, four or more pharmaceutical compositions, or five or more pharmaceutical composition, as disclosed herein. The plurality of pharmaceutical compositions can be provided simultaneously or sequentially. Thus, if a subject is to be treated with, for example, a preparation of uncultured fecal bacteria and two bacterial isolates, a first composition can comprise two of the bacterial isolates and the second composition can comprise the preparation of uncultured fecal bacteria. In a different example, if a subject is to be treated with a preparation of uncultured fecal bacteria and two bacterial isolates, a first composition can comprise the preparation of uncultured fecal bacteria in combination with (or “spiked” with) a first bacterial isolate, and a second composition can comprise the second bacterial isolate. In a different example, if a subject is to be treated with a preparation of uncultured fecal bacteria and three bacterial isolates, a first composition can comprise the first bacterial isolate, a second composition can comprise the second bacterial isolate, a third composition can comprise the third bacterial isolate, and a fourth composition can comprise the preparation of uncultured fecal bacteria.

In one aspect, a method comprises administering a pharmaceutical composition orally, by enema, or via rectal suppository. In one aspect, a pharmaceutical composition is formulated as a geltab, pill, microcapsule, capsule, or tablet. In one aspect, a pharmaceutical composition is formulated as an enteric coated capsule or microcapsule, acid-resistant capsule or microcapsule, or formulated as part of or administered together with a food, a food additive, a dairy-based product, a soy-based product or a derivative thereof, a jelly, or a yogurt. In another aspect, a pharmaceutical composition is formulated as an acid-resistant enteric coated capsule. A pharmaceutical composition can be provided as a powder for sale in combination with a food or drink. A food or drink can be a dairy-based product or a soy-based product. In another aspect, a food or food supplement contains enteric-coated and/or acid-resistant microcapsules containing a pharmaceutical composition.

Further provided herein are kits comprising any herein-disclosed pharmaceutical composition and instructions for use. For example, a kit can include one or more unit dosage forms comprising one or more bacterial mixtures. Such a kit could include for example one or more pharmaceutical compositions comprising a bacterial mixture (e.g., comprising a preparation of fecal bacteria), and optionally a delivery device to administer the composition to the subject, and instructions for administering the dosage to a subject via an appropriate delivery route. In some cases, the dosage form comprises any suitable form of live bacteria (fresh, frozen, lyophilized, etc.) and is formulated for administration to a human subject orally, by nasogastric tube, by colonoscopy, or anally. As described herein, dosage forms suitable for kits provided herein include, without limitation, liquid solutions, capsules, tablets, powders, granules, and lyophilized forms.

The instructions of a kit can describe, for example, dosing information of the one or more pharmaceutical compositions in the kit. As examples, the frequency of administration and dose of a composition, e.g., the number of capsules of a pharmaceutical composition to be administered at a given time, and the number of times of administration per day/week). In an aspect in which the kit comprises more than one composition (e.g., multiple bacterial mixtures or an additional pharmaceutical agent lacking a bacterial mixture), the instructions can describe the dosing of each composition. For example, one composition can be administered before another composition, e.g., sequential administration of the two pharmaceutical compositions separated by minutes, hours, days, weeks, months, or longer. Alternately, two compositions can be administered simultaneously.

In a further aspect, provided herein is use of a bacterial mixture described herein for manufacture of a medicament for treating a disorder described herein or for reducing the severity of one or more symptoms of a disorder described herein.

The present disclosure provides for the following embodiments:

### Embodiment 1

A method, comprising: receiving a value of a microbiome disruption index (MDI) corresponding to a fecal microbiota of a subject, wherein said MDI is representative of a divergence in microbial diversity between said fecal microbiota and a fecal microbiota of a healthy individual; and administering a therapeutic composition comprising live non-pathogenic fecal bacteria to said subject based on said value of said MDI.

### Embodiment 2

The method of embodiment 1, wherein said divergence in microbial diversity is representative of a divergence in alpha diversity between said fecal microbiota and said fecal microbiota of said healthy individual.

### Embodiment 3

The method of embodiment 1 or embodiment 2, wherein said divergence in alpha diversity is an average difference in alpha diversity between said fecal microbiota and multiple fecal microbiota of multiple healthy individuals.

### Embodiment 4

The method of any one of embodiments 1 to 3, wherein said divergence in microbial diversity is representative of beta diversity of said fecal microbiota relative to said fecal microbiota of said healthy individual.

### Embodiment 5

The method of embodiment 4, wherein said divergence in microbial diversity is representative of average beta diversity of said fecal microbiota relative to multiple fecal microbiota of multiple healthy individuals.

### Embodiment 6

The method of embodiment 5, wherein said MDI represents the product of said average divergence in alpha diversity and said beta diversity.

### Embodiment 7

The method of embodiment 6, wherein said MDI is greater than 1.

### Embodiment 8

The method of embodiment 6, wherein said alpha diversity incorporates a measure of Shannon's diversity index.

### Embodiment 9

The method of embodiment 6, wherein said beta diversity incorporates a measure of Jensen-Shannon divergence.

### Embodiment 10

The method of any one of embodiments 1 to 9, wherein said healthy individual is a donor of said live non-pathogenic fecal bacteria.

### Embodiment 11

The method of any one of embodiments 1 to 10, wherein said subject has a gut dysbiosis.

### Embodiment 12

The method of embodiment 11, wherein said MDI is diagnostic of said gut dysbiosis.

### Embodiment 13

The method of embodiment 12, wherein said gut dysbiosis is caused by a disorder or condition of said subject.

### Embodiment 14

The method of embodiment 13, wherein said disorder or condition is selected from the group consisting of Acne, AIDS Enteropathy, AIDS-related Gastroenteritis, Alopecia Totalis, Alzheimers Disease, Amyloidosis, Amyotrophic Lateral Sclerosis, Ankylosing Spondylitis, Anorexia, Antibiotic Associated Colitis, Asbergers Syndrome, Attention Deficit Disorder (ADD), Attention Deficit Hyperactivity Disorder (ADHD), Autism Spectrum Disorder (ASD), Behcet's Syndrome, Chronic Clostridium difficile Infection (CDI), Chronic constipation, Chronic Depression, Chronic Fatigue Syndrome (CFS), Chronic Idiopathic Pseudo Obstructive Syndrome, Chronic Inflammation Demyelinating Polyneuropathy, Chronic Nausea, Chronic Urticaria, Coeliac Disease, Collagenous Colitis, Colonic Polyps, Constipation Predominant FBD, Crohn's Disease, Cryptogenic Cirrhosis, Cyclic Vomiting, Dermatitis Herpetiformis, Diabetes, Familial Mediterranean Fever, Fatty Liver, Functional Bowel Disease (FBD), Gastro-oesophageal Reflux, Gillian-Barre Syndrome, Glomerulonephritis, Haemolytic Uraemic Syndrome, Halitosis, IBS constipation-predominant, IBS diarrhea/constipation alternating, IBS diarrhea-predominant, IBS pain-predominant, Idiopathic Thrombocytopenic Purpura (ITP), Idiopathic/Simple Constipation, Indeterminate Colitis, Inflammatory Bowel Disease (IBD), Irritable bowel syndrome (B S), Juvenile Diabetes Mellitus, Lyme Disease, Manic Depressive Illness, Metabolic Syndrome, Microscopic Colitis, Migraine, Mixed Cryoglobulinaemia, Mucous Colitis, Multiple Sclerosis, Myasthenia Gravis, NASH (Nonalcoholic Steatohepatitis), Non-Rheumatoid Arthritis, Non-Rheumatoid Factor Positive Arthritis, Non-ulcer Dyspepsia, Norwalk Viral Gastroenteritis, Obesity, Obsessive Compulsive Disorder, Pain Predominant FBD, Parkinson's Disease, Polyarteritis, Polyposis Coli, Primary Biliary Cirrhosis, Primary Clostridium difficile Infection (CDI), Primary Sclerosing Cholangitis (PSC), Pseudomembranous Colitis, Psychotic Disorders, Reiter's Syndrome, Relapsing Diverticulitis, Rett Syndrome, Rheumatoid Arthritis, Rosacea, Rotavirus Gastroenteritis, Sacroiliitis, Schizophrenia, Scleroderma, Sjogren's Syndome, Small Bowel Bacterial Overgrowth, Sudden Infant Death Syndrome (SIDS), Systemic Lupus Erythematosus, Ulcerative Colitis, Upper Abdominal FBD, Vasculitic Disorders, Viral Gastroenteritis, pre-diabetic syndrome, type I diabetes, type II diabetes, depression, schizophrenia, a mood disorder, an autoimmune disorder, an infection, an allergy or atopy, a neurological disorder, Vancomycin Resistant Enterococci (VRE) infection, and Methicillin Resistant Staphylococcus Aureus (MRSA) infection.

### Embodiment 15

The method of embodiment 12, wherein said gut dysbiosis is due to treatment of said subject with one or more antibiotics.

### Embodiment 16

The method of embodiment 15, wherein said one or more antibiotics comprises vancomycin.

### Embodiment 17

The method of any one of embodiments 1 to 16, wherein said fecal microbiota is a first fecal microbiota from a first stool sample of said subject.

### Embodiment 18

The method of embodiment 17, further comprising receiving a second value of an MDI corresponding to a second fecal microbiota of said subject, wherein said second value of said MDI is representative of a difference in microbial diversity between said second fecal microbiota and a fecal microbiota of a healthy individual, wherein said second fecal microbiota is from a second stool sample of said subject collected after said first stool sample.

### Embodiment 19

The method of embodiment 18, wherein said first and second stool samples are collected before said pharmaceutical composition is administered.

### Embodiment 20

The method of embodiment 19, wherein said MDI of said second fecal microbiota is greater than said MDI of said first fecal microbiota.

### Embodiment 21

The method of embodiment 20, wherein said pharmaceutical composition is administered based on the difference between said MDI of said first and second fecal microbiota.

### Embodiment 22

The method of embodiment 18, wherein said second stool sample is collected after said pharmaceutical composition is administered.

### Embodiment 23

The method of embodiment 22, wherein said MDI of said second fecal microbiota is less than said MDI of said first fecal microbiota.

### Embodiment 24

A method of treating a subject having an infectious disease, an autoimmune disease, an allergic disease, or a neurological disease, the method comprising: administering a therapeutic composition comprising live non-pathogenic fecal bacteria to said subject, wherein said administering is based on a value of a microbiome disruption index (MDI) corresponding to a fecal microbiota of said subject, wherein said MDI is representative of a difference in microbial diversity between said fecal microbiota and a fecal microbiota of a healthy individual.

### Embodiment 25

A method of manufacturing a pharmaceutical composition comprising live non-pathogenic fecal bacteria of a stool donor, said method comprising: obtaining a value of a microbiome disruption index (MDI) corresponding to a fecal microbiota of said stool donor, wherein said MDI is indicative of a microbial diversity of said fecal microbiota relative to a microbial diversity of a fecal microbiota of a healthy individual; and incorporating said live non-pathogenic fecal bacteria into said pharmaceutical composition based on said value of said MDI.

### Embodiment 26

The method of embodiment 25, wherein said MDI incorporates an indication of an alpha diversity of said fecal microbiota relative to an alpha diversity of said fecal microbiota of said healthy individual.

### Embodiment 27

The method of embodiment 26, wherein said indication comprises an average divergence in alpha diversity between said fecal microbiota and multiple fecal microbiota of multiple healthy individuals.

### Embodiment 28

The method of any one of embodiments 25 to 27, wherein said MDI incorporates a beta diversity of said fecal microbiota relative to said fecal microbiota of said healthy individual.

### Embodiment 29

The method of embodiment 28, wherein said MDI incorporates an average beta diversity of said fecal microbiota relative to multiple fecal microbiota of multiple healthy individuals.

### Embodiment 30

The method of embodiment 29, wherein said MDI represents the product of said average divergence in alpha diversity and said beta diversity.

### Embodiment 31

The method of embodiment 30, wherein said MDI is less than 1.

### Embodiment 32

The method of embodiment 30, wherein said alpha diversity incorporates a measure of Shannon's diversity index.

### Embodiment 33

The method of embodiment 30, wherein said beta diversity incorporates a measure of Jensen-Shannon divergence.

### Embodiment 34

The method of any one of embodiments 25 to 33, wherein said live non-pathogenic fecal bacteria are obtained from multiple stool samples of said stool donor.

### Embodiment 35

A method, comprising: receiving a value of a microbiome disruption index (MDI) corresponding to a fecal microbiota of a subject, wherein said MDI is representative of a divergence in microbial diversity between said fecal microbiota and a fecal microbiota of a healthy individual; and administering a therapeutic composition comprising live non-pathogenic fecal bacteria to said subject based on said value of said MDI.

### Embodiment 36

A method of treating a subject having a disorder related to a dysbiosis of the intestinal microbiota of said subject, said method comprising: determining a divergence in alpha diversity of a fecal microbiota of said subject and a fecal microbiota of a healthy individual; determining a beta diversity of said fecal microbiota of said subject relative to a fecal microbiota of a second healthy individual; obtaining a microbiome disruption index (MDI) from said divergence in alpha diversity and said beta diversity; and administering a therapeutic composition comprising live non-pathogenic fecal bacteria to said subject based on said value of said MDI.

### Embodiment 37

The method of embodiment 36, wherein said MDI is the product of said divergence in alpha diversity and said beta diversity.

### Embodiment 38

The method of embodiment 36 or embodiment 37, wherein said healthy individual and said second healthy individual are the same individual.

### Embodiment 39

The method of embodiment 36 or embodiment 37, wherein said healthy individual and said second healthy individual are different individuals.

### Embodiment 40

The method of any one of embodiments 36 to 39, wherein said divergence in alpha diversity is an average difference in alpha diversity between said fecal microbiota of said subject and multiple fecal microbiota of multiple healthy individuals.

### Embodiment 41

The method of any one of embodiments 36 to 40, wherein said beta diversity is an average beta diversity of said fecal microbiota of said subject relative to multiple fecal microbiota of multiple healthy individuals.

### Embodiment 42

The method of embodiment any one of embodiments 36 to 41, wherein said MDI is greater than 1.

### Embodiment 43

The method of any one of embodiments 36 to 42, wherein said alpha diversity incorporates a measure of Shannon's diversity index.

### Embodiment 44

The method of any one of embodiments 36 to 43, wherein said beta diversity incorporates a measure of Jensen-Shannon divergence.

### Embodiment 45

The method of any one of embodiments 36 to 44, wherein at least one of said healthy individual and said second healthy individual is a donor of said live non-pathogenic fecal bacteria.

### Embodiment 46

A method of decolonizing antibiotic-resistant bacteria from the intestine of a subject in need thereof, comprising: administering live non-pathogenic fecal bacteria to said subject based on detection of a greater relative abundance of bacteria from the Proteobacteria phylum in a fecal microbiota of said subject compared to a fecal microbiota of a healthy individual.

### Embodiment 47

The method of embodiment 46, wherein said antibiotic-resistant bacteria comprise Vancomycin-Resistant Enterococcus.

### Embodiment 48

The method of embodiment 47, wherein said bacteria from the Proteobacteria phylum comprise Enterobacteriaceae.

### Embodiment 49

The method of embodiment 48, wherein said bacteria from the Proteobacteria phylum consist of Enterobacteriaceae.

### Embodiment 50

The method of embodiment 49, wherein said healthy individual is not colonized with Vancomycin-Resistant Enterococcus.

### Embodiment 51

The method of any one of embodiments 1 to 23, wherein said subject is undergoing or is in need of hematopoietic stem cell transplantation (HSCT).

### Embodiment 52

The method of any one of embodiments 1 to 23 and 51, wherein said value of said MDI and/or said second value of said MDI identifies said patient as at risk for a bloodstream infection.

### Embodiment 53

A method of treating a disorder related to a gut dysbiosis in a patient in need thereof, the method comprising: administering to said patient a therapeutic composition comprising live non-pathogenic fecal bacteria; following said administering, receiving a value of a microbiome disruption index (MDI) corresponding to a fecal microbiota of said subject, wherein said MDI is representative of a divergence in microbial taxonomic diversity between said fecal microbiota of said subject and a fecal microbiota of one or more healthy subjects, wherein said one or more healthy subjects do not have said disorder; and based on a value of said MDI, administering a second dose of said therapeutic composition to said subject.

### Embodiment 54

The method of embodiment 53, wherein said MDI is representative of a divergence in bacterial taxonomic diversity between said fecal microbiota of said patient and said fecal microbiota of said one or more healthy subjects.

### Embodiment 55

The method of embodiment 53 or embodiment 54, wherein said divergence in microbial diversity is representative of a divergence in alpha diversity between said fecal microbiota of said patient and said fecal microbiota of said one or more healthy subjects.

### Embodiment 56

The method of embodiment 55, wherein said divergence in alpha diversity represents an average difference in alpha diversity between said fecal microbiota of said patient and multiple fecal microbiota of multiple healthy subjects.

### Embodiment 57

The method of embodiment 56, wherein said divergence in microbial diversity is representative of beta diversity of said fecal microbiota of said patient relative to said fecal microbiota of multiple healthy subjects.

### Embodiment 58

The method of embodiment 57, wherein said MDI represents the product of said average difference in alpha diversity and said beta diversity.

### Embodiment 59

The method of any one of embodiments 53 to 58, wherein said therapeutic composition is administered when said value of said MDI is greater than 1.

### Embodiment 60

The method of any one of embodiments 53 to 59, wherein said preparation of live non-pathogenic fecal bacteria comprises uncultured bacteria from a stool of a healthy donor.

### Embodiment 61

The method of any one of embodiments 53 to 60, wherein said preparation of live non-pathogenic fecal bacteria comprises cultured bacteria.

### Embodiment 62

The method of any one of embodiments 53 to 61, wherein said patient is undergoing hematopoietic stem cell transplantation (HSCT).

### Embodiment 63

The method of embodiment 62, wherein said value of said MDI identifies said patient as at risk for a bloodstream infection.

### Embodiment 64

The method of any one of embodiments 53 to 63, wherein said disorder is selected from the group consisting of Acne, AIDS Enteropathy, AIDS-related Gastroenteritis, Alopecia Totalis, Alzheimers Disease, Amyloidosis, Amyotrophic Lateral Sclerosis, Ankylosing Spondylitis, Anorexia, Antibiotic Associated Colitis, Asbergers Syndrome, Attention Deficit Disorder (ADD), Attention Deficit Hyperactivity Disorder (ADHD), Autism Spectrum Disorder (ASD), Behcet's Syndrome, Chronic Clostridium difficile Infection (CDI), Chronic constipation, Chronic Depression, Chronic Fatigue Syndrome (CFS), Chronic Idiopathic Pseudo Obstructive Syndrome, Chronic Inflammation Demyelinating Polyneuropathy, Chronic Nausea, Chronic Urticaria, Coeliac Disease, Collagenous Colitis, Colonic Polyps, Constipation Predominant FBD, Crohn's Disease, Cryptogenic Cirrhosis, Cyclic Vomiting, Dermatitis Herpetiformis, Diabetes, Familial Mediterranean Fever, Fatty Liver, Functional Bowel Disease (FBD), Gastro-oesophageal Reflux, Gillian-Barre Syndrome, Glomerulonephritis, Haemolytic Uraemic Syndrome, Halitosis, IBS constipation-predominant, IBS diarrhea/constipation alternating, IBS diarrhea-predominant, IBS pain-predominant, Idiopathic Thrombocytopenic Purpura (ITP), Idiopathic/Simple Constipation, Indeterminate Colitis, Inflammatory Bowel Disease (IBD), Irritable bowel syndrome (B S), Juvenile Diabetes Mellitus, Lyme Disease, Manic Depressive Illness, Metabolic Syndrome, Microscopic Colitis, Migraine, Mixed Cryoglobulinaemia, Mucous Colitis, Multiple Sclerosis, Myasthenia Gravis, NASH (Nonalcoholic Steatohepatitis), Non-Rheumatoid Arthritis, Non-Rheumatoid Factor Positive Arthritis, Non-ulcer Dyspepsia, Norwalk Viral Gastroenteritis, Obesity, Obsessive Compulsive Disorder, Pain Predominant FBD, Parkinson's Disease, Polyarteritis, Polyposis Coli, Primary Biliary Cirrhosis, Primary Clostridium difficile Infection (CDI), Primary Sclerosing Cholangitis (PSC), Pseudomembranous Colitis, Psychotic Disorders, Reiter's Syndrome, Relapsing Diverticulitis, Rett Syndrome, Rheumatoid Arthritis, Rosacea, Rotavirus Gastroenteritis, Sacroiliitis, Schizophrenia, Scleroderma, Sjogren's Syndome, Small Bowel Bacterial Overgrowth, Sudden Infant Death Syndrome (SIDS), Systemic Lupus Erythematosus, Ulcerative Colitis, Upper Abdominal FBD, Vasculitic Disorders, Viral Gastroenteritis, pre-diabetic syndrome, type I diabetes, type II diabetes, depression, schizophrenia, a mood disorder, an autoimmune disorder, an infection, an allergy or atopy, a neurological disorder, Vancomycin Resistant Enterococci (VRE) infection, and Methicillin Resistant Staphylococcus Aureus (MRSA) infection.

### EXAMPLES

### Example 1: Retrospective Study

Patients from six different centers are evaluated (Table 1). Patients receive either an allogenic FMT from a universal stool donor (provided by OpenBiome) or an autologous FMT as a control. Stool samples are collected from patients prior to FMT and at one or more visits post-FMT. The primary endpoint is defined as recurrence of infection at 8 weeks. The exact time frame of the sample collections varied at different sites, but almost all patients have one sample between the FMT and 6-weeks post-FMT.

### Example 2: Antibiotic Resistance Testing

Stool samples are sent to OpGen for antibiotic resistance testing, which is performed using OpGen's Acuitas MDRO gene test. This is a multiplex PCR test for common MDRO genes including VRE, CRE, and ESBL-E associated genes.

### Example 3: Sample Collection and 16S rRNA Sequencing

Patients collect stool samples by sub-sampling approximately 1 gram of formed stool or 1 mL of liquid stool into 5 mL of RNALater. Samples are kept at room temperature for up to one week before being aliquotted and stored at −80 C. Samples are thawed, RNALater is removed with PBS washing, and approximately 200 mg pelleted sample was aliquoted into 96 well Qiagen PowerBead Plates. DNA extraction, PCR amplification of the 16S rDNA V4 region, and Illumina paired-end sequencing is performed.

### Example 4: 16S Processing

Primers are trimmed, paired ends merged, and operational taxonomic units (OTUs) identified with a custom pipeline. In order to have maximum resolution for engraftment analysis, OTUs are defined by unique 16S sequences. OTUs represented in fewer than two unique samples and samples with fewer than 100 remaining reads are discarded. Taxonomic assignments for each OTU are called using UTAX trained on the Green genes 13_5 97% database. On average, there are 31,128+13,316 reads per sample in the final OTU table.

### Example 5: Data Analysis

Most data analysis is performed using in house python code, with the exception of the group significance test, which is performed using Qiime. The Alpha and beta diversity calculations are done using in house code and the Scikit-Bio python package. Alpha diversity is calculated using the Shannon Index. Beta diversity is calculated using the Jensen-Shannon Divergence. The MDI for a sample is calculated by multiplying the average difference in alpha diversity (calculated using log 2) between the sample and the healthy cohort by the average beta diversity between the sample and the healthy cohort. Based on the approximate range of MDI observed, in healthy stool donors, a healthy MDI score is defined as being between 0 and 1. A dysbiotic MDI score is greater than 1, based on the calculation of the MDI for the patients with CDI infections and using a publicly-available dataset consisting of patients undergoing chemotherapy and antibiotic treatment (Y. Taur et al., Clin Infect Dis, vol. 55, no. 7, pp. 905-914, 2012). ROCs and AUCs are calculated and visualized using the Scikit-Learn python package.

### Example 6: FMT Prevents rCDI Recurrence and Decolonizes VRE

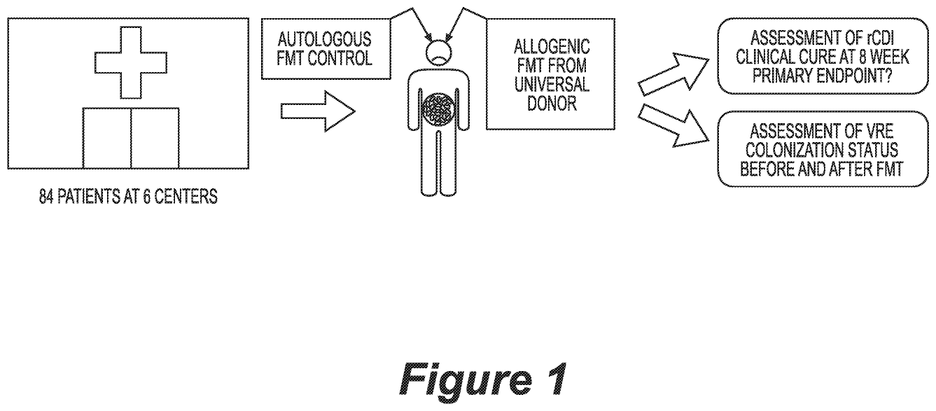

Our multi-center retrospective analysis (FIG. 1) includes stool samples collected from 84 patients with rCDI that are enrolled in six independent studies (Table 1). Using samples from different studies is advantageous because the use of multiple studies minimizes artefactual effects observed in the data due to geography or method of collection. These patients either receive allogenic FMT from an OpenBiome universal donor (n=65) or autologous FMT as a placebo treatment (n=19). Stool samples are sequenced from before and up to two samples from after they receive FMT using the 16S sequencing methods described above. Samples are tested for presence of Vancomycin-Resistant Enterococcus (VRE) and other Antibiotic Resistant Bacteria (ARB)using the Opgen Acuitas® MDRO Gene Test, a validated multiplex PCR test, and colonization is defined as a positive result at one or more dilutions. The primary endpoint for clinical cure of rCDI is defined as prevention of infection recurrence at 8 weeks. The primary endpoint for VRE colonization is defined as clearance of VRE colonization at the first follow-up visit (first follow-up visit is less than 6 weeks after FMT for all patients).

TABLE 1
Samples from six independent studies are screened for VRE colonization

| | | | After | | | |
|---|---|---|---|---|---|---|
| | | | exclu- | | | |
| | | | sions | | | Samples |
| | | | for | | | receiving |
| | | After | avail- | | Samples | autolo- |
| Principal | | exclusions | able | | receiving | gous |
| investi- | Total | for severe | time- | VRE + | allogenic | FMT |
| gator | patients | CDI | points | samples | FMT | (control) |
| Brandt | 18 | 18 | 16 | 5 | 1 | 4 |
| Rodriguez | 17 | 13 | 13 | 1 | 1 | 0 |
| Kelly | 21 | 21 | 21 | 6 | 3 | 3 |
| Allegretti | 17 | 16 | 16 | 4 | 4 | 0 |
| Grinspan | 7 | 6 | 6 | 2 | 2 | 0 |
| Fischer | 4 | 4 | 4 | 1 | 1 | 0 |
| Total | 84 | 78 | 76 | 19 | 12 | 7 |

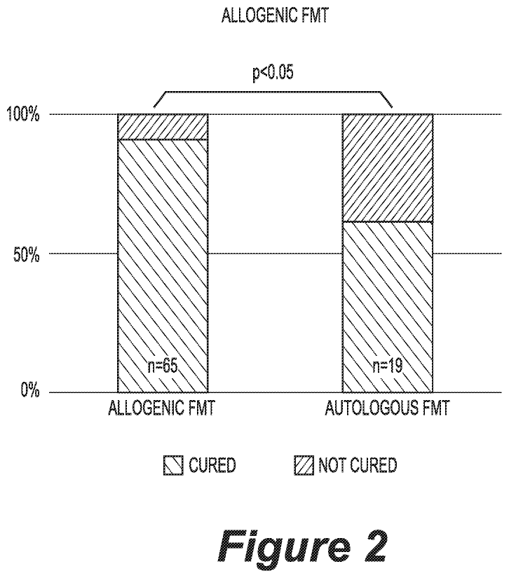

FMT is an effective therapy for rCDI in this trial (FIG. 2). Of the 65 patients who receive allogenic FMT, 59 (91%) achieve the primary endpoint of lack of recurrence at 8 weeks after the first dose. After removing patients with severe or severe/complicated CDI, as the nature of the disease at that state is significantly different, there are 58 patients who receive FMT and 53 (91%) are cured at the primary endpoint. In contrast, of the 19 control group patients who receive autologous FMT, only 12 (63%) are clinically cured at the primary endpoint. There are no patients with severe or severe-complicated CDI in the control group. The difference between allogenic and autologous FMT constitutes a statistically significant difference (p<0.05 by Fisher's exact test).

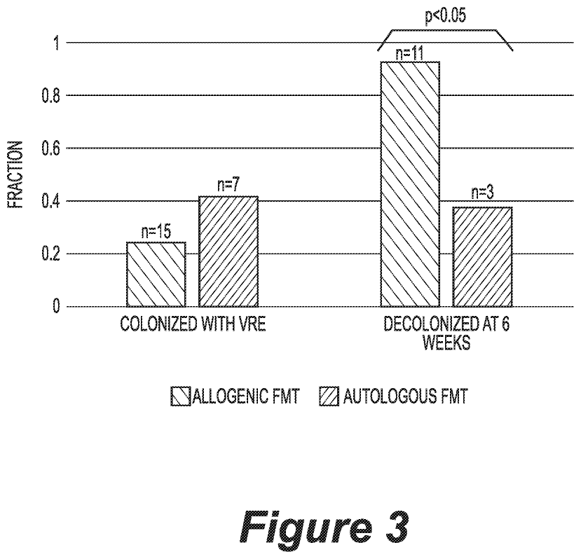

The majority of patients colonized with VRE are also decolonized after FMT. Of 65 patients, 15 patients (23%) in the allogenic FMT group and 7 of 19 (37%) patients in the placebo group are colonized with VRE at baseline (pre-FMT). 3 of 15 (20%) of the VRE positive patients in the allogenic FMT group have severe or severe/complicated CDI and are excluded from subsequent analyses because of the significant physiological differences between standard and severe CDI, bringing the number of VRE positive patients receiving allogenic FMT to 12. At the primary endpoint (6 weeks post FMT) 11 of 12 (92%) colonized patients in the FMT group test VRE negative compared to 3 of 7 (43%) in the control group (FIG. 3). The difference between allogenic and autologous FMT constitutes a statistically significant difference (p<0.05 by Fisher's exact test).

### Example 7: Development of a Microbiome Disruption Index that Classifies Patients with CDI

Retrospective data is used to develop a microbiome disruption index (MDI). The goal of the MDI is to use broad descriptors of the microbiome community (alpha and beta diversity), so that the broad descriptors can be used to identify different types of dysbiosis in different populations. Samples from the retrospective study and samples from a healthy population, 63 of OpenBiome's universal stool donors, to calculate the MDI. OpenBiome's stool donors are extensively screened for pathogens, immune disorders, and other disorders that may be associated with changes in the gut microbiome.

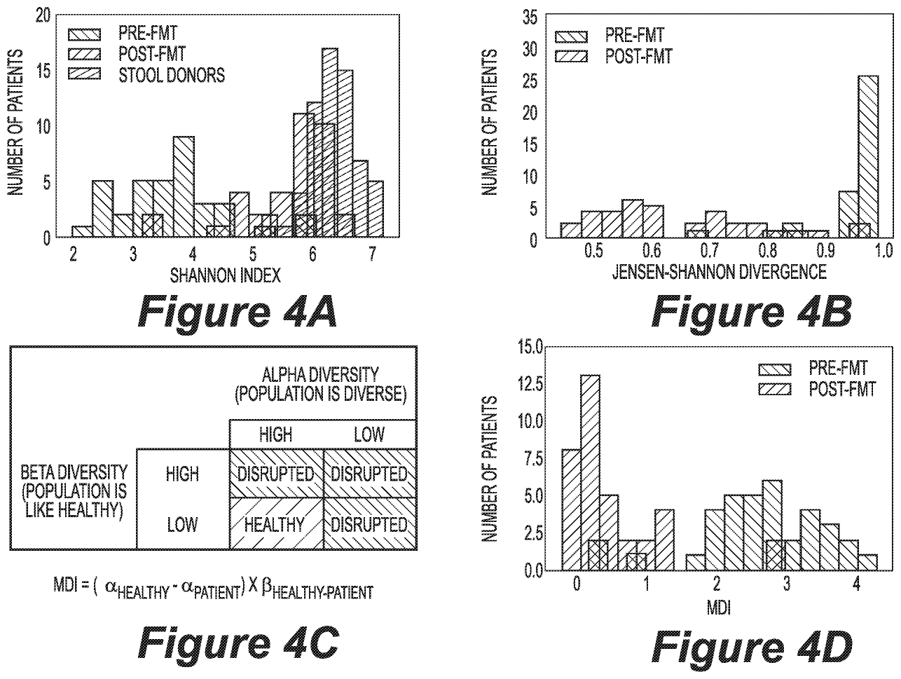

First, the alpha diversity of these populations are compared because alpha diversity is decreased in CDI patients and known to increase after FMT. Shannon's diversity index, a quantitative measure of total species richness that is commonly used with microbial datasets and robust to differences in sequencing depth, is utilized. The Shannon index is calculated for each sample and donor, and the average difference in alpha diversity between the sample and each of the donors is used to describe the change from healthy levels of diversity. Because dysbiotic guts can be dominated by one or a handful of high abundance species, we would expect that the diversity or species richness would be lower in patients pre-FMT than post-FMT and in healthy OpenBiome stool donors. This is shown in FIG. 4A. Donors are much more diverse than pre-FMT patients, and post-FMT patients have a Shannon index that more closely resembled donors than patients pre-FMT.

A difference in the populations of patients pre-FMT and post-FMT using only alpha diversity has been observed. However, there have been cases described where patients with significant dysbiosis have had high alpha diversity but a composition significantly different from that of a healthy person. Therefore, a measure of beta diversity in the MDI is also included. The Jensen-Shannon divergence (JSD) is a method for assessing the distance between two probability distributions used to quantify differences between human microbiota communities. This method is used to look at the community divergence between patients with rCDI and healthy OpenBiome stool donors. Patient samples are collected before and after FMT, and the JSD is calculated for each sample and donor combination. The average JSD between the sample and each of the donors represents the average difference between the sample and a healthy community. Post-FMT, patients more closely resemble the donors (FIG. 4B).

Both alpha and beta diversity are combined into the final MDI (FIG. 4C). To combine these two measures, the average difference between alpha diversity of a sample and that of the healthy population is calculated. Then, the calculated value is multiplied by the average beta diversity between the sample and the healthy population. Calculating MDI this way identifies different types of community disruptions and is on a simple scale that generally ranges from 0-5 (FIG. 4). Using this and other published datasets, samples from healthy people usually have an MDI between 0 and 1, while those with a disrupted microbiome due to antibiotic treatment or infection have an MDI greater than 1. Indeed, in this cohort, the majority of samples taken before FMT had an MDI greater than 1, and the majority of samples taken after FMT had an MDI less than 1 (FIG. 4D).

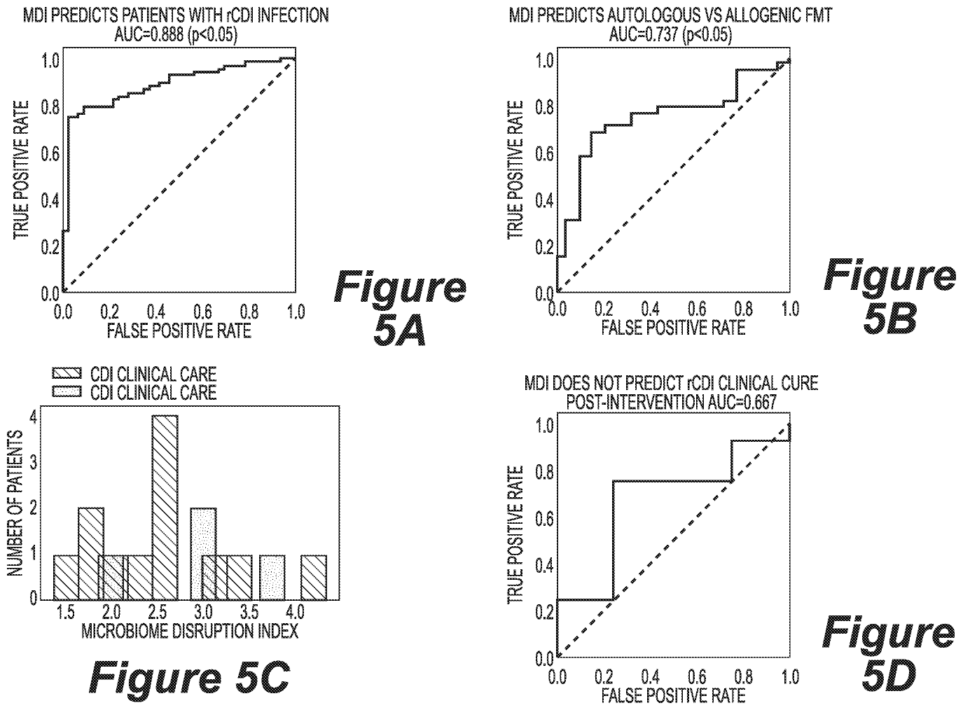

To determine if MDI is able to classify patients with CDI, a receiver operator curve (ROC) is utilized. Some of the post-FMT patients (both in the allogenic and autologous groups) still have CDI after the intervention. The ROC plots the true positive rate of the model by the false positive rate of the model and calculates the area under the curve (AUC). A model that does no better than chance has an AUC of 0.5, and a perfect model has an AUC of 1. The MDI is able to predict which samples are from patients with CDI very accurately, with an AUC greater than 0.8 (FIG. 5A). The MDI is also able to predict which patients have received allogenic vs autologous FMT, with an AUC greater than 0.7 (FIG. 5B). Because some of the autologous patients are cured of CDI at this point, this suggests that the community of a patient cured of CDI with autologous FMT differs from that of a community of a patient cured with allogenic FMT.

The MDI can predict which patients would be cured using the same method described above. Briefly, The MDI of these two patient populations is plotted as a histogram and very little difference is found between patients who became cured and those who do not (FIG. 5C), and as expected, this is reflected in a low AUC value (FIG. 5D). A low AUC value suggests that there are likely other differences in these populations, extrinsic to their microbiome, that are driving whether patients respond to therapy. Alternatively, this global metric may not capture the relevant microbiome differences. Furthermore, this analysis is complicated by the fact that FMT is such a successful therapy that there are very few samples in the unsuccessful cure section making it challenging to identify differentiating features within the two groups.

### Example 8: Prediction of VRE Colonization State

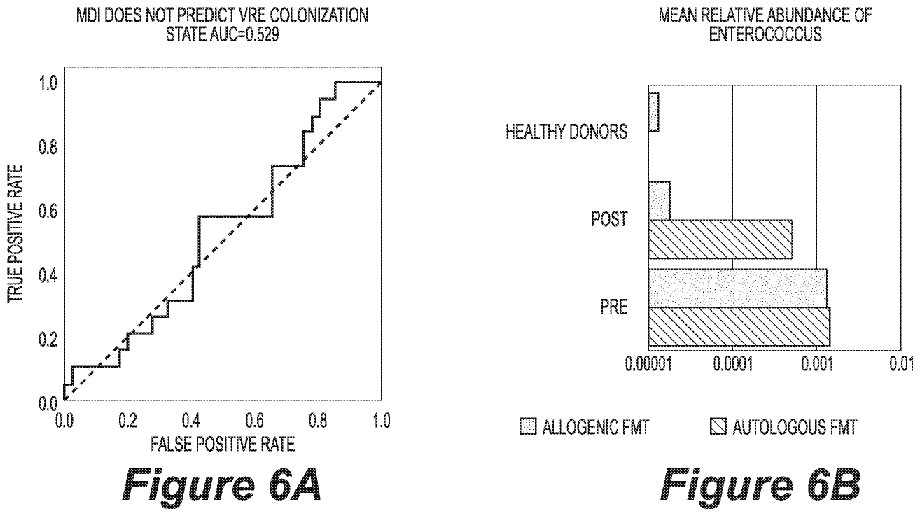

Patients with C. difficile infection are already experiencing severe disruption of the gut microbiota. To determine whether patients who are co-colonized with VRE experience additional disruption of the microbiome, patient samples from before they received either intervention are compared. The comparison is based on the MDI of samples pre-FMT. A strong prediction of whether patients were colonized with VRE is not determined (FIG. 6A). Though there is little difference in the extent of the dysbiosis as measured by the MDI between subjects with rCDI and those with rCDI and colonized with VRE, it cannot be concluded that other populations have no difference in community disruption between those colonized with VRE and those not colonized. In this light, it is more informative to compare those colonized and not colonized post-allogenic FMT when CDI is cured, but with only one subject colonized after allogenic FMT in this dataset, a meaningful comparison cannot be made.

Previous work in different patient populations shows that antibiotic treatment and subsequent VRE colonization can result in Enterococcal domination of the gut and a significant decrease in diversity, and whether this is also the case in this population is tested. So, the data are examined to assess the relative abundance of the Enterococcus genus across the different samples. The total relative abundance of all Enterococcus strains is measured in the gut of the VRE positive patients before and after intervention. It is impossible to identify VRE using 16S sequencing because the vancomycin resistance gene vanA is not sequenced and because it is difficult to distinguish Enterococcus species using only the 16S gene (for example E. casseliflavus and E. gallinarum have 99.9% identical 16S sequences), so Enterococcus abundance is used as a proxy. Enterococcus abundance increase in VRE colonized samples compared to healthy donors (FIG. 6B). Furthermore, though there is little difference between the abundance of Enterococcus before FMT, the average relative abundance of Enterococcus in the post autologous FMT groups is higher than in the post allogenic FMT group (FIG. 6B). Both groups exhibit a decrease in abundance, however, so antibiotic treatment and/or the return to health caused by resolution of the C. difficile infection may have played a role in the decrease in Enterococcal abundance.

### Example 9: Specific Taxa are Associated with VRE Colonization

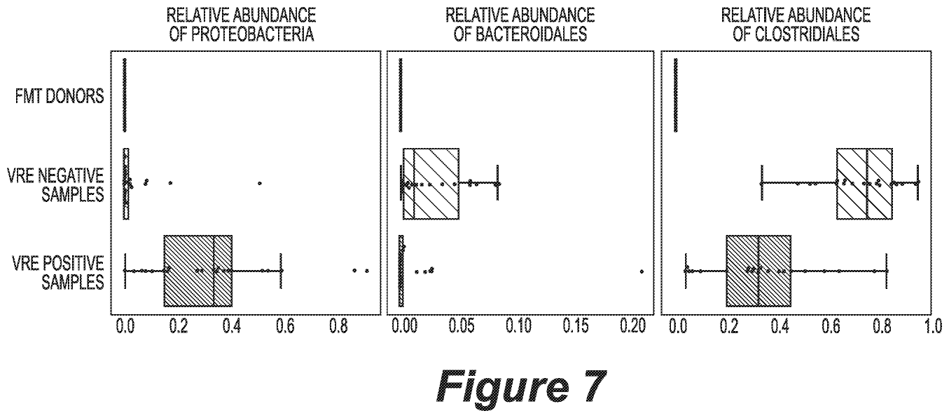

Because the MDI does not predict VRE decolonization, it is tested whether there are specific bacterial taxa whose presence or relative abundance could better predict decolonization. VRE positive and VRE negative samples pre-intervention and post-intervention are compared separately to control for the community differences associated with FMT. Qiime's group significance tool is used to identify OTUs that are significantly different between groups, and a number of OTUs are identified that are significantly enriched in the VRE colonized samples, though none are significantly enriched after multiple hypothesis correction (Table 2). These OTUs fall into three taxonomic groups: Proteobacteria (specifically beta- and gamma-Proteobacteria), Bacteroidales, and Clostridiales (Table 3), and Proteobacterial abundance is generally increased in the VRE positive samples compared to the VRE negative samples and healthy donors in separate group significance tests (FIG. 7). In fact, the increased Proteobacterial abundance in VRE positive samples, is quite striking, with the relative abundance of Proteobacteria exceeding 80% in some of the samples.

TABLE 2
Taxa found to be more abundant in VRE colonized
samples pre and post intervention

| | | Pre or | Fold increased | |
|---|---|---|---|---|
| | | Post | abundancein | |
| | | inter- | VRE-colonized | |
| Family | Genus | vention | samples | p-value |
| Lachnospiraceae | Lachnospira | Pre | 454 | 0.001 |
| Rikenellaceae | N/A | Pre | 9.48 | 0.003 |
| Bacteroidaceae | Bacteroides | Pre | 168 | 0.007 |
| Enterobacteriaceae | N/A | Pre | 3.78 | 0.021 |
| Enterobacteriaceae | Escherichia | Pre | 8.53 | 0.025 |
| Neisseriaceae | Neisseria | Pre | 110 | 0.038 |
| Porphyromonadaceae | Parabacteroides | Pre | 104 | 0.041 |
| Tissierellaceae | Finegoldia | Post | 27.8 | 0.008 |
| Unnamed | N/A | Post | 13.3 | 0.004 |
| Burkholderiales | | | | |
| Enterobacteriaceae | N/A | Post | 7.26 | 0.016 |

TABLE 3
The differentially abundant taxa fall into three groups

| | Number of | Average fold increased |
|---|---|---|
| | differentially | abundance in |
| Group | abundant otus | VRE-colonized samples |
| Phylum Proteobacteria | 4 | 28.6 |
| Order Bacteroidales | 3 | 93.8 |
| Order Clostridiales | 2 | 241 |

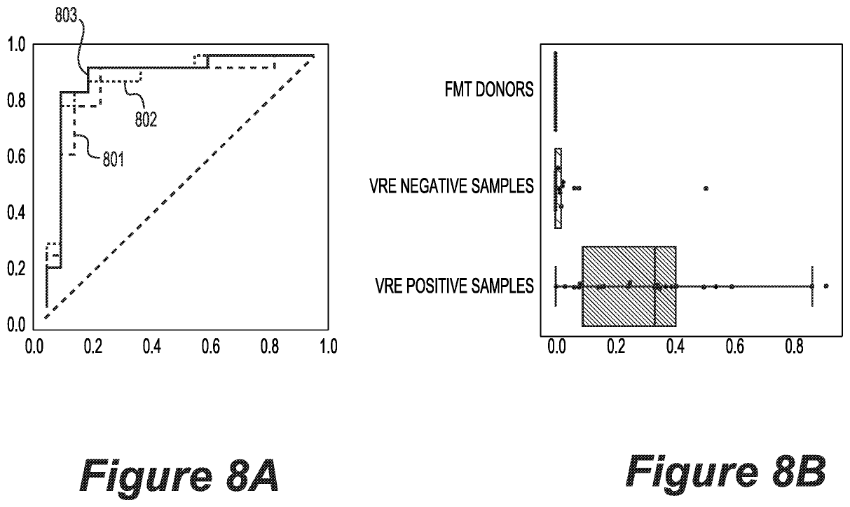

Specific clades associated with a disorder or disease can be used in the future to predict whether a patient has that disease or is likely to develop that disease. Therefore, it is tested whether Proteobacterial abundance is able to classify subjects colonized with VRE pre-FMT. A receiver operator curve is again used for this analysis and it is found that Proteobacterial abundance is quite predictive with an AUC of 0.893 (FIG. 8A). This is quite predictive, but Proteobacteria is a whole phylum, and there may be clades within that phylum that are even better predictors. Therefore, the predictive ability is assessed for the taxa of the significantly enriched OTUs identified in the group significance analysis. These taxa (Escherichia, Neisseria, unnamed Enterobacteriaceae, and unnamed Burholderiales) when combined, are slightly better predictors that all Proteobacteria with an AUC of 0.919 (FIG. 8A). Because two of these four taxa belong to the Enterobacteriaceae family, it is also tested whether the Enterobacteriaceae family alone is predictive of VRE colonization, and it is, with a slightly higher AUC of 0.924 (FIG. 8A-B). This suggests that the majority of the predictive effect seen from Proteobacteria is actually due to the relative abundance of strains belonging to the Enterobacteriaceae family. In the future, patients can be screened for increased Enterobacteriaceae abundance in their stool, to identify whether they are at risk for VRE or other pathogen colonization.

### Example 10: MDI Predicts which Patients Will Develop a Bloodstream Infection

To confirm that the MDI calculation would be useful in other datasets, published data from Taur et al. AIMS Microbiology, 5(1): 1-18 (2012) are used to calculate the MDI. In this dataset, the authors collect stool samples longitudinally from 94 patients undergoing hematopoietic stem cell transplantation (HSCT), while also tracking clinical data such as antibiotic use and development of bloodstream infections.

If there is a way to identify which patients are at highest risk of developing bloodstream infection, those patients can be treated with extra care, put into isolation, or treated with an FMT to decolonize pathogens and decrease their risk of an infection. Therefore, for each patient, the stool sample that is immediately before the stem cell transplant is identified and the MDI of that sample is calculated, using the same database of stool donors for the comparator as used in Example 7. Then, the MDI of the patients that do or do not go on to develop a bloodstream infection is compared, and while the MDI of these two populations overlaps, the MDI for almost all those patients that go on to develop bloodstream infection is almost always greater than 1: the previously-defined maximum cutoff for a healthy MDI (FIG. 3A). When confirmed with an ROC plot, an AUC of >0.7 is calculated (FIG. 3B), suggesting that one can predict relatively accurately the patients that are at the highest risk of infection. This is especially exciting considering that in some cases, infection can be identified weeks before the infection actually developed. In summary, it is shown that the MDI can be applied outside of FMT/C. difficile datasets, and the MDI can be used to identify the patients at highest risk of bloodstream infection. See also, Montassier et al. Genome Medicine (2016) 8:49. This result shows the robustness of the MDI method. Because differences in sample preparation, sequencing, and patient population often create artefactual differences between datasets, cross-validation of a method such as this across different cohorts and datasets is generally very challenging.

What is claimed is:

1. A method of treating a patient having a disorder associated with a gut dysbiosis, the method comprising:
receiving a value of a microbiome disruption index (MDI) corresponding to a fecal microbiota of said patient, wherein said MDI is representative of a divergence in microbial taxonomic diversity between said fecal microbiota of said patient and fecal microbiota of one or more healthy subjects, wherein said one or more healthy subjects do not have said disorder; and
administering a therapeutic composition comprising a preparation of live non-pathogenic fecal bacteria to said patient based on said value of said MDI.

2. The method of claim 1, wherein said MDI is representative of a divergence in bacterial taxonomic diversity between said fecal microbiota of said patient and said fecal microbiota of said one or more healthy subjects.

3. The method of claim 1, wherein said divergence in microbial diversity is representative of a divergence in alpha diversity between said fecal microbiota of said patient and said fecal microbiota of said one or more healthy subjects.

4. The method of claim 3, wherein said divergence in alpha diversity represents an average difference in alpha diversity between said fecal microbiota of said patient and multiple fecal microbiota of multiple healthy subjects.

5. The method of claim 4, wherein said divergence in microbial diversity is representative of beta diversity of said fecal microbiota of said patient relative to said fecal microbiota of multiple healthy subjects.

6. The method of claim 5, wherein said MDI represents the product of said average difference in alpha diversity and said beta diversity.

7. The method of claim 6, wherein said therapeutic composition is administered when said value of said MDI is greater than 1.

8. The method of claim 1, wherein said preparation of live non-pathogenic fecal bacteria comprises uncultured bacteria from a stool of a healthy donor.

9. The method of claim 1, wherein said preparation of live non-pathogenic fecal bacteria comprises cultured bacteria.

10. The method of claim 1, wherein said patient is undergoing hematopoietic stem cell transplantation (HSCT).

11. The method of claim 10, wherein said value of said MDI identifies said patient as at risk for a bloodstream infection.

12. The method of claim 1, wherein said disorder is selected from the group consisting of Acne, AIDS Enteropathy, AIDS-related Gastroenteritis, Alopecia Totalis, Alzheimers Disease, Amyloidosis, Amyotrophic Lateral Sclerosis, Ankylosing Spondylitis, Anorexia, Antibiotic Associated Colitis, Asbergers Syndrome, Attention Deficit Disorder (ADD), Attention Deficit Hyperactivity Disorder (ADHD), Autism Spectrum Disorder (ASD), Behcet's Syndrome, Chronic Clostridium difficile Infection (CDI), Chronic constipation, Chronic Depression, Chronic Fatigue Syndrome (CFS), Chronic Idiopathic Pseudo Obstructive Syndrome, Chronic Inflammation Demyelinating Polyneuropathy, Chronic Nausea, Chronic Urticaria, Coeliac Disease, Collagenous Colitis, Colonic Polyps, Constipation Predominant FBD, Crohn's Disease, Cryptogenic Cirrhosis, Cyclic Vomiting, Dermatitis Herpetiformis, Diabetes, Familial Mediterranean Fever, Fatty Liver, Functional Bowel Disease (FBD), Gastro-oesophageal Reflux, Gillian-Barre Syndrome, Glomerulonephritis, Haemolytic Uraemic Syndrome, Halitosis, IBS constipation-predominant, IBS diarrhea/constipation alternating, IBS diarrhea-predominant, IBS pain-predominant, Idiopathic Thrombocytopenic Purpura (ITP), Idiopathic/Simple Constipation, Indeterminate Colitis, Inflammatory Bowel Disease (IBD), Irritable bowel syndrome (IBS), Juvenile Diabetes Mellitus, Lyme Disease, Manic Depressive Illness, Metabolic Syndrome, Microscopic Colitis, Migraine, Mixed Cryoglobulinaemia, Mucous Colitis, Multiple Sclerosis, Myasthenia Gravis, NASH (Nonalcoholic Steatohepatitis), Non-Rheumatoid Arthritis, Non-Rheumatoid Factor Positive Arthritis, Non-ulcer Dyspepsia, Norwalk Viral Gastroenteritis, Obesity, Obsessive Compulsive Disorder, Pain Predominant FBD, Parkinson's Disease, Polyarteritis, Polyposis Coli, Primary Biliary Cirrhosis, Primary Clostridium difficile Infection (CDI), Primary Sclerosing Cholangitis (PSC), Pseudomembranous Colitis, Psychotic Disorders, Reiter's Syndrome, Relapsing Diverticulitis, Rett Syndrome, Rheumatoid Arthritis, Rosacea, Rotavirus Gastroenteritis, Sacroiliitis, Schizophrenia, Scleroderma, Sjogren's Syndome, Small Bowel Bacterial Overgrowth, Sudden Infant Death Syndrome (SIDS), Systemic Lupus Erythematosus, Ulcerative Colitis, Upper Abdominal FBD, Vasculitic Disorders, Viral Gastroenteritis, pre-diabetic syndrome, type I diabetes, type II diabetes, depression, schizophrenia, a mood disorder, an autoimmune disorder, an infection, an allergy or atopy, a neurological disorder, Vancomycin Resistant Enterococci (VRE) infection, and Methicillin Resistant Staphylococcus Aureus (MRSA) infection.

13. A method, comprising:
administering a therapeutic composition comprising a preparation of live non-pathogenic fecal bacteria to a patient having a disorder associated with a gut dysbiosis;
receiving a value of a microbiome disruption index (MDI) corresponding to a fecal microbiota of said patient following said administering, wherein said MDI is representative of a divergence in microbial taxonomic diversity between said fecal microbiota of said patient and fecal microbiota of one or more healthy subject that do not have said disorder; and
administering a second dose of said therapeutic composition to said patient based on said value of said MDI being greater than a threshold value.

14. The method of claim 13, wherein said divergence in microbial diversity is representative of a divergence in alpha diversity between said fecal microbiota of said patient and said fecal microbiota of said one or more healthy subjects.

15. The method of claim 14, wherein said divergence in microbial diversity is representative of beta diversity of said fecal microbiota of said patient relative to said fecal microbiota of one or more healthy subjects.

16. The method of claim 15, wherein said MDI represents the product of said average difference in alpha diversity and said beta diversity.

17. The method of claim 16, wherein said threshold value is 1.

18. The method of claim 13, wherein said preparation of live non-pathogenic fecal bacteria comprises uncultured bacteria from a stool of a healthy donor.

19. The method of claim 13, wherein said preparation of live non-pathogenic fecal bacteria comprises cultured bacteria.

20. A method of treating a subject having a disorder, the method comprising administering to said subject a therapeutic composition comprising a preparation of uncultured fecal bacteria, wherein said administering is based on a value of a microbiome disruption index (MDI) corresponding to a fecal microbiota of said subject, wherein said MDI is representative of a divergence in bacterial diversity between said fecal microbiota and fecal microbiota of one or more healthy individuals, wherein said disorder is selected from the group consisting of an infectious disease, an autoimmune disease, an allergic disease, and a neurological disease, and wherein said one or more healthy individuals do not have said disorder.
